(19) United States
(12) Patent Application Publication
(10) Pub. No.: US 2008/0233101 A1
(43) Pub. Date: Sep. 25, 2008

(54) RNA-mediated epigenetic regulation of gene transcription

(75) Inventor: Frank Sauer, Riverside, CA (US)

Correspondence Address: BEYER WEAVER LLP, P.O. BOX 70250, OAKLAND, CA, 94612-0250, US

(73) Assignee: The Regents of the University of California

(21) Appl. No.: 11/520,494

(22) Filed: Sep. 12, 2006

Related U.S. Application Data
(60) Provisional application No. 60/718,257, filed on Sep. 15, 2005.
(60) Provisional application No. 60/741,014, filed on Nov. 29, 2005.

(51) Int. Cl. A61K 38/47; C12N 5/06; C12Q 1/68; C07H 21/04

(52) U.S. Cl. 424/94.61; 435/375; 435/366; 435/6; 536/24.1

(57) ABSTRACT

The invention provides a method of regulating transcription of a gene that is a target for an epigenetic regulator; a method of characterizing the transcriptional activity of such a gene; a method of screening for a chromosomal element (CE) for an epigenetic regulator of a target gene; an isolated complex including an epigenetic regulator for a target gene, wherein the epigenetic regulator is specifically bound to a non-coding polynucleotide; and a method of screening for a modulator of transcription of a gene that is a target for an epigenetic regulator.

### CROSS-REFERENCE TO RELATED APPLICATIONS

This application claims the benefit of the following U.S. provisional applications: Ser. No. 60/718,257, filed Sep. 15, 2005 and Ser. No. 60/741,014, filed Nov. 29, 2005. Each of the applications cited above is incorporated by reference in its entirety.

### STATEMENT AS TO RIGHTS TO INVENTIONS MADE UNDER FEDERALLY SPONSORED RESEARCH AND DEVELOPMENT

This invention was made with government support under grant no. R01GM073776. The Government may have certain rights in the invention.

### FIELD OF THE INVENTION

This invention relates generally to epigenetic regulation of gene transcription. More particularly, the invention provides methods and compositions related to the regulation of transcription of a gene that is a target for an epigenetic regulator that acts via a non-coding polynucleotide that is encoded by and binds to a chromosomal element in the target gene.

### BACKGROUND OF THE INVENTION

Metazoan organisms consist of myriad genetically identical but structurally and functionally heterogeneous cells. The developmental fate of cells is established during development and mitotically propagated throughout the entire life cycle. Cell fate determination requires the establishment and maintenance of specific gene expression programs. To accomplish mitotic inheritance of gene expression patterns cells have evolved specialized mechanisms, termed epigenetics (1-3).

Among the key players in epigenetics is the phylogenetically highly conserved protein family of epigenetic regulators (4). On the basis of their transcriptional regulatory potential, epigenetic regulators have been subdivided into two groups. Members of the trithorax-group (trxG) of epigenetic activators maintain transcriptionally active transcription states, while members of the Polycomb-group (PcG) of epigenetic repressors maintain repressed transcription states (4). Extensive efforts have revealed that many epigenetic regulators control gene expression at the level of chromatin by establishing transcriptional competent or silent chromatin structures (5,6). Among the epigenetic regulators are chromatin-remodeling ATPases (such as Drosophila Brahma and Mi-2), whose activities contribute to the mitotic inheritance of active or silent gene expression states by altering the position of nucleosomes, the smallest structural entity within chromatin (5). Other epigenetic regulators exert their regulatory activity by mediating the posttranslational modification of histones (H1, H2A, H2B, H3, H4), the basic building blocks of nucleosomes (6). Several epigenetic activators (Trx, Trr, Ash1) and repressors [E(Z)] are lysine-specific histone methyltransferases (HMTs) that contain an enzymatic module (SET-module) consisting of the SET-domain and flanking cysteine-rich regions. Methylation of lysine residues in H3 and H4 has been correlated with epigenetic activation and repression (6). One hallmark of epigenetic repression is the methylation of lysines 9 (H3-K9) and 27 (H3-K27) in histone H3 (7,8). In contrast, epigenetic activation has been linked to methylation of lysine 4 in H3 (H3-K4) (4,6).

The epigenetic activator “absent small and homeotic discs” (Ash1) promotes transcriptional activation by establishing a trivalent histone methylation pattern (Ash1 histone methylation pattern) consisting of tri-methylated H3-K4, H3-K9 and lysine 20 in H4 (H4-K20) (9). Although ubiquitously expressed, Ash1 maintains activated gene expression states preferentially in larval imaginal discs that give rise to the appendages such as legs, wings and haltere in the adult fly (10,11). For example, Ash1 is essential for the expression of the homeotic gene Ultrabithorax (Ubx) in 3rd-leg and haltere imaginal discs, and Ubx expression coincides with the placement of the Ash1 histone methylation pattern (9,10). Noteworthy, although expressed in all imaginal discs, Ash1 activates Ubx expression only in a specific subset of imaginal discs, indicating that the target recognition and transcriptional activity of Ash1 is cell-type specific. However, the molecular mechanisms controlling the cell type-specific transcriptional activity of Ash1 were previously unknown.

Epigenetic regulators are recruited to specific chromosomal elements (CEs) that are present in the cis-regulatory region of target genes (2-4). The same CE can act as an activating or a silencing module (4). In the repressed state, the CEs represent “Polycomb response elements” (PREs) and facilitate the recruitment of PcG proteins (12,13). In the activated state, CEs function as “trithorax response elements” (TREs) and mediate binding of trxG proteins (12,13). CEs transcribe non-coding RNA (ncRNA) in a pattern that is identical to that of the protein-encoding gene, whose activity they control (12,13). Genetic experiments demonstrate that the transcription of CEs switches silent PREs into active TREs, which indicates that the transcription of CEs plays an important role in epigenetic activation and silencing (4,12,13). Current models propose that transcription renders CEs accessible to trxG proteins. However, how transcription of CEs culminates into the recruitment of trxG regulators was unknown.

Only 4 of the identified epigenetic regulators were known to bind specific DNA sequences in target genes and many of the epigenetic regulators, including the HMTs, lack classical DNA binding domains (14,15). Consequently, it remained unknown how epigenetic HMTs and other epigenetic activators bind target genes in general and in a cell-type specific fashion. Thus, the dissection of the molecular mechanisms that mediate and confine target gene recognition of epigenetic regulators to specific cells lies at the heart of the epigenetics field. The work described herein answers the key question of how epigenetic regulators without known DNA binding capabilities recognize and bind target genes in chromatin.

### SUMMARY OF THE INVENTION

The invention provides a method of regulating transcription of a gene that is a target for an epigenetic regulator. The gene includes a cis-regulatory region including a chromosomal element (CE) for the epigenetic regulator, and the CE includes a sequence that is a template for a non-coding polynucleotide. The method entails contacting cells including the gene and the epigenetic regulator with an effective amount of a modulator. The modulator the modulator alters the level of: (1) the non-coding polynucleotide; (2) the specific binding of the non-coding polynucleotide to the target gene; and/or (3) the specific binding of the epigenetic regulator to the non-coding polynucleotide. An effective amount of a modulator according to the invention is an amount sufficient to regulate transcription of the gene.

In one embodiment of the method, the cells include mammalian cells. In a variation of this embodiment, the mammalian cells include human cells.

In particular embodiments of the method, the gene that is a target for the epigenetic regulator includes a homeotic gene. Exemplary homeotic genes include Ultrabithorax (Ubx), abdominal B (abd-B), wingless (wg), Sex-combs reduced (SCR), Antennapedia (ANTP), a Hox gene, and orthologs thereof.

In specific embodiments of the method, the epigenetic regulator includes a histone methylransferase. The regulator can be one including a SET-module. In particular embodiments, the epigenetic regulator activates transcription of the target gene. Exemplary epigenetic activators include Trithorax (Trx), Trithorax-related (Trr), absent small and homeotic discs (Ash1), human Trx, human Ash1, human Ash2, Mixed Lineage Leukemia (MLL), MLL-related (MLL-1, MLL-2, MLL-3, MLL-4, MLL-5), ALL-1, ALL-2, ALL-3, ALL-4, ALL-5, and orthologs thereof. Alternatively, the epigenetic regulator can repress transcription of the target gene. Exemplary epigenetic repressors include D. melanogaster Enhancer of Zeste (E(Z)), Polycomb (PC), Medusa (Mdu), Su(var)3-5, Su(var)3-7, Su(var)3-9, Su(var)3-6, Su(var)2-1, Su(var)2-10, Su(var)3-3, mammalian Enhancer of Zeste (EZH2), M33, SETDB1, ENX-2, SUV39H1, SUV39H2, and orthologs thereof.

In particular embodiments of the method, the non-coding polynucleotide includes non-coding RNA.

The method can be carried out using a modulator that reduces the level of: (1) the non-coding polynucleotide; (2) the specific binding of the non-coding polynucleotide to the target gene; and/or (3) the specific binding of the epigenetic regulator to the non-coding polynucleotide. Thus, for example, the epigenetic regulator can include a transcriptional activator, in which case, the modulator represses transcription of the target gene. Alternatively, the epigenetic regulator can include a transcriptional repressor, in which case the modulator activates transcription of the target gene.

In other embodiments, the method is carried out using a modulator that increase the level of: (1) the non-coding polynucleotide; (2) the specific binding of the non-coding polynucleotide to the target gene; and/or (3) the specific binding of the epigenetic regulator to the non-coding polynucleotide. In this instance, if the epigenetic regulator includes a transcriptional activator, the modulator activates transcription of the target gene. Alternatively, if the epigenetic regulator includes a transcriptional repressor, the modulator represses transcription of the target gene.

The transcriptional regulation method of the invention can be carried out on cells in vitro or in vivo.

In certain embodiments, the modulator modulates cell proliferation and/or cell differentiation. In exemplary embodiments, the modulator can be contacted with a cell selected from the group consisting of a cancer cell, a stem cell, and a dormant cell. Thus, for example, the cell can be a stem cell, and the transcription of one or more genes that is/are a target for one or more epigenetic regulators is regulated to induce the stem cell to differentiate. In exemplary in vivo embodiments, a modulator that modulates cell proliferation and/or cell differentiation is contacted with cells by administering a composition including the modulator to a subject having a condition treatable by modulation of cell proliferation and/or cell differentiation. The subject can, for example, be a patient having a condition selected from: cancer, neurodegenerative disease, paralysis, diabetes, burn, tissue failure, organ failure, osteoporosis, muscular dystrophy, and wound. Such conditions can also be treated by removing the cells from a patient having such a condition, contacting the cells with the modulator ex vivo, and then reimplanting the cells into the patient.

Another aspect of the invention is a method of characterizing the transcriptional activity of a gene that is a target for an epigenetic regulator in a biological sample including the gene and the epigenetic regulator. The gene includes a cis-regulatory region including a chromosomal element (CE) for the epigenetic regulator, and the CE includes a sequence that is a template for a non-coding polynucleotide. The method entails determining whether the non-coding polynucleotide is present in the biological sample. In preferred embodiments, the method additionally includes determining whether the non-coding polynucleotide is physically associated with the CE and the epigenetic regulator. The determination of whether the non-coding polynucleotide is physically associated with the CE and the epigenetic regulator can be carried out by in vivo cross-linked chromatin immunoprecipitation. In particular embodiments, the amount of non-coding polynucleotide physically associated with the CE and the epigenetic regulator in a test sample is compared with the amount of non-coding polynucleotide physically associated with the CE and the epigenetic regulator in a control sample.

In one embodiment, the transcriptional activity of the target gene is correlated with an abnormal condition, and the non-coding polynucleotide is detected as an indicator of the abnormal condition. In a variation of this embodiment, the abnormal condition includes abnormal cell proliferation. In exemplary embodiments of this type, the difference between the amount of non-coding polynucleotide physically associated with the CE and the epigenetic regulator in a test sample, compared with the amount of non-coding polynucleotide physically associated with the CE and the epigenetic regulator in a control sample, provides a metric useful in the diagnosis and/or prognosis of cancer.

In a second embodiment, the transcriptional activity of the target gene is correlated with a cell type, and the non-coding polynucleotide is detected as an indicator of the cell type.

In a third embodiment, the transcriptional activity of the target gene is correlated with a stage of cell differentiation, and the non-coding polynucleotide is detected as an indicator of that stage.

The method of characterizing transcriptional activity can be carried out using cells, target genes (e.g., homeotic genes), epigenetic regulators (e.g., histone methylransferases, regulators including a SET-module), and non-coding polynucleotides as described above for the transcriptional regulation method.

The invention also provides a method of screening for a chromosomal element (CE) for an epigenetic regulator of a target gene, wherein the CE includes a sequence that is a template for a non-coding polynucleotide. In one embodiment, the method entails determining whether a sequence of a putative CE is transcribed in a cell.

In particular embodiments, the putative template for the non-coding polynucleotide is identified by sequence comparison with a CE selected from tre1 (SEQ ID NO:2), tre2 (SEQ ID NO:3), and tre3 (SEQ ID NO:4).

In a second embodiment, the method entails determining whether the epigenetic regulator is physically associated with a non-coding polynucleotide corresponding to a putative CE and/or physically associated with the putative CE. In a variation of this embodiment, the method determines whether this physical association exists in a cell. This variation can be carried out, for example, using in vivo cross-linked chromatin immunoprecipitation.

In a third embodiment, the method entails determining whether a non-coding polynucleotide corresponding to a putative CE mediates transcriptional regulation by the epigenetic regulator. This embodiment can be carried out, for example, by measuring transcriptional regulation directly by assaying transcription of the target gene. Alternatively, transcriptional regulation can be measured indirectly by assaying a biological response that is correlated with transcription of the target gene.

The method of screening for a CE can be carried out using cells, target genes (e.g., homeotic genes), epigenetic regulators (e.g., histone methylransferases, regulators including a SET-module), and non-coding polynucleotides as described above for the transcriptional regulation method. In preferred embodiments, screening for a CE is performed using cells in vitro.

Another aspect of the invention is an isolated complex including an epigenetic regulator for a target gene, wherein the epigenetic regulator is specifically bound to a non-coding polynucleotide. The gene includes a cis-regulatory region including a chromosomal element (CE) for the epigenetic regulator, and the CE includes a sequence that is a template for a non-coding polynucleotide. Suitable target genes (e.g., homeotic genes), epigenetic regulators (e.g., histone methylransferases, regulators including a SET-module), and non-coding polynucleotides include those described above for the transcriptional regulation method.

Also provided by the invention is a method of screening for a modulator of transcription of a gene that is a target for an epigenetic regulator. The gene includes a cis-regulatory region including a chromosomal element (CE) for the epigenetic regulator, and the CE includes a sequence that is a template for a non-coding polynucleotide. The method entails: (a) contacting a test agent with a mixture or cell including the non-coding polynucleotide and the CE and/or the epigenetic regulator, and (b) detecting the ability of the test agent to modulate specific binding of the non-coding polynucleotide to the CE and/or the epigenetic regulator. In preferred embodiments, the contacting is carried out in vitro. Any specific binding is preferably compared with specific binding in the absence of test agent or in the presence of a lower amount of test agent than in (a). In particular embodiments, the determination of specific binding includes in vivo cross-linked chromatin immunoprecipitation. The method can additionally include recording any test agent that specifically modulates said specific binding in a database of candidate agents that may modulate transcription of the gene. The method can also, optionally, include determining whether the test agent modulates cell proliferation and/or differentiation.

Screening for a transcriptional modulator can be carried out using target genes (e.g., homeotic genes), epigenetic regulators (e.g., histone methylransferases, regulators including a SET-module), and non-coding polynucleotides such as those described above for the transcriptional regulation method.

### BRIEF DESCRIPTION OF THE DRAWINGS

FIG. 1(A-C). The TREs of Ubx are transcribed in a cell-type specific fashion. (A) Schematic representation of the Ubx locus (top) and the bxd DNA-element (bottom). The positions of bxd, Ubx promoter (P), TREs, spacer DNA (S-1. S-2, N, and S-3) are indicated. Arrows indicate the orientation and relative length of TRE transcripts detected by RACE. (B) Photographs of RT-PCR assays detecting the transcripts of the indicated bxd elements and control transcripts (actin5C, Ubx) in RNA pools isolated from imaginal discs (3rd leg, haltere, and wing), and Schneider S2 cells (S2 cells), and genomic DNA (genomic). (C) PCR analysis of XChIP immunoprecipitates detecting the association of Ash1 with Ubx TREs in imaginal discs, and S2 cells. In vivo cross-linked chromatin was immunoprecipitated using antibodies to Ash1. PCR analyses detected the presence TRE-1, TRE-2 and TRE-3 in immunoprecipitated DNA pools. Input represents the amount of transcripts or TREs detected in 0.5% of the starting material.

FIG. 2(A-C). Recruitment of Ash1 to Ubx TREs in 3rd leg imaginal discs. (A) PCR analysis of XChIP immunoprecipitates detecting the association of Ash1 and the presence of the Ash1 histone methylation pattern at the TREs and promoter of Ubx in 3rd leg imaginal discs isolated from wild-type (WT) and ash1²² mutant Drosophila 3rd instar larvae. In vivo cross-linked chromatin was immunoprecipitated using antibodies to Ash1, rat and rabbit anti-serum (control), tri-methylated H3-K4 (tri-meH3-K4), tri-methylated H3-K9 (tri-meH3-K9), and tri-methylated H4-K20 (tri-meH4-K20). Input represents the amount of TRE-1 detected in 0.5% of the starting material. (B) XChIP analysis as described in (A) except that chromatin was immunoprecipitated using an antibody to di-methylated H3-K9 (di-meH3-K9). (C) RT-PCR analysis detecting the transcripts of bxd elements in reversed transcribed RNA pools isolated from wild-type (WT) and ash1²² mutant 3rd leg imaginal discs or in genomic DNA (G).

FIG. 3(A-C). The SET-domain of Ash1 associates with TRE transcripts in vitro. (A) Autoradiograms of in vitro protein-RNA binding assays. Radiolabeled sense (+) and anti-sense (+) transcripts of TRE-1, TRE-2, N, and TRE-3 were incubated with anti-FlagM2 antibody agarose (Flag beads), or Flag-beads loaded with recombinant Ash1 SET or Medusa (Mdu). After incubation, beads were precipitated and washed. Retained RNA was purified and separated by native PAGE using 4% polyacrylamide gels. (B) Schematic representation of Ash1 and truncated Ash1-derivatives. The position of the SET-domain (SET) and PRE- and POST-SET domains (P) are indicated. (C) In vitro protein-RNA binding assays as in (A) except that Flag-beads were loaded with Ash1SET (amino acids 1001-1619), Ash 1 DN (amino acids 1001-2218), Ash1C (amino acids 1619-2218), or Ash1N (amino acids 1-1001). (A,C) Input represents 10% of the input RNA.

FIG. 4(A-C). The association of Ash1 with TREs is RNA-dependent. (A) Photographs of PCR analysis detecting the association of Ash1 with bxd transcripts in cross-linked chromatin isolated from 3rd leg imaginal discs. Native chromatin was isolated from 3rd leg discs, sheared and treated with recombinant BSA (mock) or RNase-A, RNAse-H, RNase-III. Treated chromatin was cross-linked, sheared, and immunoprecipitated with antibodies to Ash1 or rat serum (control). The precipitated RNA was purified and reverse transcribed. PCR detected the presence of TRE transcripts in generated cDNA pools. (B) Photographs of XChIP assays detecting the association of Ash1 with TREs in chromatin. XChIP was performed as described in (A) except that precipitated DNA was purified. PCR detected the presence of bxd DNA elements and the Ubx promoter in immunoprecipitates (C) XChIP assays as in (B) except that chromatin was immunoprecipitated by using antibodies to TBP and PCR detected the presence of the Ubx promoter (Ubx-P) and string/cdc25 promoter (string-P) in precipitated DNA pools. (A-C) Input represents DNA and RNA detected in 0.5% of the input RNA.

FIG. 5(A-D). TRE transcripts mediate the recruitment of Ash1 to Ubx TREs in 3rd leg imaginal discs. (A) Photographs of PCR analysis detecting the association of Ash1 with the indicated bxd DNA elements in mock and RNase-treated chromatin isolated from 3rd leg imaginal discs. Native chromatin was isolated from 3rd leg discs, sheared and treated with recombinant BSA (mock), RNase-A, RNAse-H, or RNase-III. Treated chromatin was immunoprecipitated with antibodies to Ash1, TBP or rat serum (control). The precipitated RNA was purified and reverse transcribed. PCR detected the presence of TRE transcripts, control transcripts and Ubx in generated cDNA pools. (B) Photographs of NChIP assays detecting the association of Ash1 with TRE transcripts in native chromatin. NChIP was performed as described in (A) except that immunoprecipitated RNA was purified. RT-PCR detected the presence of TRE-transcripts. (C) Photographs of NChIP assays detecting the association of Ash1 with TRE transcripts in chromatin and the soluble, histone-free nuclear extract. NChIP was performed as described in (A) except that native chromatin or soluble nuclear extract were used as starting material. RT-PCR monitored the presence of TRE transcripts in immunoprecipitated RNA pools. (D) RT-PCR analysis of XChIP RNA immunoprecipitates detecting the chromatin-associated bxd transcripts (top) and corresponding bxd DNA templates (bottom) in chromatin isolated from wild-type (WT) and ash 122 mutant 3rd-leg discs. Chromatin was immunoprecipitated by using antibodies to di-methylated H3-K9 (di-meH3-K9) or rat serum (C). Precipitated RNA or DNA was purified. RT-PCR and PCR detected the transcripts and corresponding DNA elements of bxd in immunoprecipitates. (A-D) Input represents the amount of TREs and TRE transcripts detected in 0.5% of the starting material.

FIG. 6(A-G). TRE transcripts reconstitute the association of Ash1 with Ubx TREs and Ubx transcription in S2 cells. (A) Photograph of PCR analysis detecting TRE transcripts and actin5C transcription in wild-type S2 cells (−) and S2 cells transfected with plasmids transcribing mdu (mock), sense TRE transcripts [TRE1(+), TRE2(+), TRE3(+)], or anti-sense TRE transcripts [TRE1(−), TRE2(−), TRE3(−)]. (B) PCR assays as in (A) but detecting Ubx transcription wild type and transfected S2 cells. (C) PCR analysis of XChIP immunoprecipitates detecting the association of Ash1 with the Ubx TREs in S2 cells transcribing mdu (mock), sense TRE transcripts or anti-sense TRE transcripts. In vivo cross-linked chromatin was immunoprecipitated using antibodies to Ash1. Precipitated DNA was purified. PCR detected the presence of Ubx TREs in precipitated DNA pools. (D,E) Photographs of XChIP assays detecting the association of Ash1 with TRE transcripts (D) and TREs or the Ubx promoter (Ubx-P) (E) in chromatin and the soluble, histone-free nuclear extract of S2 cells transiently co-transcribing TRE(1+), TRE2(+) and TRE3(+). XChIP was performed as described in (C) except that precipitated RNA (D) and DNA (E) were purified. RT-PCR monitored the presence of TRE transcripts in immunoprecipitates (D). PCR monitored the presence of TREs and the Ubx-promoter in precipitated DNA pools (E). (F,G) Photographs of chromatin immunoprecipitation assays as in (D,E) except that native chromatin was used.

FIGS. 7(A-B). (A) Coomasssie blue stained SDS polyacrylamid gel showing recombinant truncated Ash1-derivatives and Medusa (Mdu) used for in vitro protein-RNA interaction assays (FIG. 3). Proteins were expressed in Sf9 cells infected with recombinant baculovirus expressing Flag-tagged proteins. Recombinant proteins were immunoprecipitated using anti-Flag(M2) antibody agarose. Immunoprecipitated proteins (arrowheads) were electrophoretically separated on 8% SDS-polyacrylamide gel. Stars indicate the position of the light and heavy chain of anti-Flag antibodies. (B) In vitro protein-RNA binding assays programmed with radiolabeled TRE1(+), TRE2(+), or TRE3(+) and Flag-beads loaded with Ash1 SET. Binding was monitored in the absence or presence of increasing amounts of competitor: TRE1(+), TRE2(+) and TRE3(+) RNA [TRE(+)]; double stranded RNA (dsTRE1(+/−) consisting of TRE1(+) and TRE1(−)′ or RNA/DNA hybrids consisting of TRE1(+) and the complementary DNA strand of TRE-1 (TRE-1-) TRE-1. Input represents 10% of the input RNA.

FIG. 8. Photographs of RT-PCR reactions detecting the association of Ash1 with TRE and control transcripts in RNA immunoprecpitates generated by XChIP. In vivo cross-linked chromatin was isolated from 3rd leg discs, sheared, and immunoprecipitated using antibodies to Ash1 or rat serum (C). Immunoprecipitated RNA was purified and reverse transcribed. PCR detected TRE transcripts, Ubx, even-skipped (eve), Antennapedia (Antp), actin5C (act5C), string/cdc25 (stg), twine (twe), CyclinE (CycE), cdc2, CyclinA (CycA), and CyclinD (CycD) in generated cDNA pools. Input represents 0.5% of RNA detected in the starting material.

FIG. 9(A-B). Cooperative activation of Ubx transcription by TRE transcripts. (A) Photographs of RT-PCR analyses detecting the transcription of Ubx, sense (+) and anti-sense (−) TRE transcripts and actin5C in RNA pools isolated from S2 cells transfected with plasmids transcribing mdu (mock) or one or multiple sense [TRE1(+), TRE2(+), TRE3(+)] or anti-sense [TRE1(−), TRE2(−), TRE3(−)] TRE transcripts. (B) PCR analyses of XChIP DNA immunoprecipitates detecting the association of Ash1 with TREs in transfected cells described in (A). In vivo-cross-linked chromatin was immunoprecipitated using antibodies to Ash1. PCR detected the presence of TREs in immunoprecipitated DNA pools. (A,B) Input represents TRE transcripts, actin5C and Ubx detected in Drosophila genomic DNA (A) and TREs present in 0.5% of the starting chromatin (B).

FIG. 10. TRE transcripts trigger Ash1 recruitment and Ash1-mediated histone methylation at the TREs of Ubx. Photographs of PCR analysis detecting Ash1 and the Ash1 histone methylation pattern at the Ubx TREs. In vivo cross-linked chromatin was isolated from S2 cells transfected with plasmids transcribing mdu (mock), TRE1(+), TRE2(+), TRE3(+) or the corresponding anti-sense transcripts [TRE1(−), TRE2(−), TRE3(−)]. Chromatin was precipitated using antibodies to Ash1 and the Ash1 histone methylation pattern (see FIG. 2). PCR detected the presence of TREs and the promoter of Ubx in precipitated DNA pools. (A,B) Input represents TREs detected in 0.5% of the starting chromatin (B).

FIG. 11. TRE transcripts specifically associate with Ubx TREs. PCR analyses of XChIP DNA immunoprecipitates detecting the association of TRE transcripts with Drosophila CEs Fab7 (82600-82900) and MCP (110100-110400), the Ubx promoter (Ubx-P), engrailed promoter (eng-P) and the iab4 element of the bithorax complex. In vivo cross-linked chromatin was isolated form S2 cells transiently transcribing TRE1(+), TRE2(+), or TRE3(+), sheared and immunoprecipitated with antibodies recognizing Ash1. PCR detected the presence of Drosophila CEs in immunoprecipitated DNA pools. Input represents amount of tested DNA elements detected in 0.5% of the starting chromatin.

FIG. 12(A-G). TRE1-RNA mediates transcription activation by Ash1 in S2 cells. (A) Photographs of ethidium bromide stained agarose gels showing the reaction products of RT-PCR experiments. RNA was isolated from S2 cells containing the stable integrated reporter ptetO7-TATA-TRE-1, S2 cells expressing TET-VP16, or ptetO7-TATA-TRE-1 cells expressing TET-VP-16. RT-PCR was used to detect the Ubx transcript [+123-(+654)] or TRE1-RNA. (B) Photographs of ethidium bromide stained agarose gels showing the reaction products of XChIP experiments. In vivo cross-linked chromatin was isolated from S2 cells described in (A). Chromatin was immunoprecipitated using antibodies recognizing Ash1 or the indicated histone modifications. PCR was used to monitor the presence of the TRE-1 element in immunoprecipitated DNA pools. (C) XCHIP as in (B) using anti-Ash1 antibody but purifying precipitated RNA. Purified RNA was reverse transcribed and used as a template for PCR to detect the presence of TRE1-RNA. (D) XChIP experiments as in (C) except that native chromatin was isolated from the indicated S2 cells (A). Chromatin was incubated with an RNase cocktail, cross-linked, sheared and immunoprecipitated using anti-Ash1 antibody. Precipitated DNA and RNA were purified. RNA was reverse transcribed. PCR was used to detect the presence of the TRE-1 element (TRE-1+RNA) or TRE1-RNA (TRE1-RNA+RNA) in the purified DNA/RNA pools. (E) RT-PCR experiments as in (A) monitoring Ubx transcription in transgenic cells transcribing the lagging strand of TRE-1, expressing TET-VP16, or expressing both. RT-PCR detected the TRE-1, Ubx and actin5C transcript [+123-(+654)] or TRE1-RNA. (F) RT-PCR experiments as in (E) except that S2 cells were used transcribing the leading strand of TRE-2 (TRE2-RNA). (G) RT-PCR experiments as in (E) except that S2 cells were used transcribing the leading strand of TRE-3 (TRE3-RNA).

FIG. 13(A-D). Miss-transcription of TRE1-RNA recruits Ash1 to Ubx in Drosophila wing imaginal discs. (A) Photographs of ethidium bromide stained agarose gels showing the reaction products of RT-PCR and XChIP experiments. RNA and in vivo cross-linked chromatin were isolated from wing imaginal discs carrying the effector gene expressing TET-VP16, the reporter gene ptetO7-TATA-TRE-1 transcribing TRE1-RNA(+) under control of the TET-VP-16 activator, or both genes. RT-PCR was used to detect Ubx and TRE-1 transcription. In vivo cross-linked chromatin was immunoprecipitated using the indicated antibodies. PCR detected the presence of Ash1 and its histone methylation pattern at the TRE-1 element of Ubx in precipitated RNA pools. (B-C) RT-PCR assays detecting the transcription of Ubx in wing imaginal discs transcribing TRE2-RNA (B) or TRE3-RNA (C). (D) Photographs of ethidium bromide stained agarose gels showing the results of in vitro protein:RNA interaction assays. The chromatin-packaged or naked reporter gene pUbx-EGFP consisting of the 26 kb Ubx enhancer fused to the reporter gene EGFP was incubated with Ash1 SET and the indicated TRE-transcripts (upper panel). XChIP using anti-Ash1 antibodies monitored the recruitment of Ash1 to the reporter.

FIG. 14(A-C). Interaction of MLL and EZH2 with TRE- and PRE-transcripts Photographs of ethidium bromide stained agarose gels showing the reaction products of RT-PCR experiments detecting TRE- (A) and PRE-transcripts (B) in embryonic mouse cDNA libraries. (C) Autoradiogram of protein:RNA interaction assays. Resin loaded with MLLSET, EZH2(SET) (+) or a resin containing Flag-TBP (−) was incubated with radiolabeled, in vitro transcribed TRE- and PRE-transcripts derived from the indicated Hox genes.

FIG. 15(A-F). MLL and EZH2 bind TRE- and PRE-transcripts. (A) Western blot analysis with anti-MLL antibodies detecting immunoprecipitated MLL in Sf9 cells expressing recombinant MLLC₁₈₀ (R), Drosophila Schneider cells (C), MEF cells (E), or MPMP cells (M. (B) Western blot analysis of proteins immunoprecipitated with anti-EZH2 antibodies from MEF cells and MEF cells expressing EZH2 [MEF(EZH2)]. (C) Photograph of RT-PCR reactions detecting TRE-transcripts generated from TRE-elements present in the indicated Hox genes. RNA was isolated from MPMP (M) and MEF (E) cells. (D) Photographs of RT-PCR (left) and XChIP (right) experiments using RNA and cross-linked chromatin isolated from wild type MPMP cells (−) or MPMP cells that had been transfected with plasmids transcribing control RNA [lacZ; (−)] or the indicated TRE-transcripts. RT-PCR detected the transcription of Hoxa9. XChIP using anti-MLL antibodies detected the interaction of MLL with Hoxa9 TRE-element. (E) RT-PCR experiments as in (C) except that RNA was prepared from MEF (E) and MEF cells expressing EZH2 (E+EZ). (F) RT-PCR (left) and XChIP (right) experiments as in (D) except that RNA and cross-linked chromatin was isolated from E+EZ cells (−) and E+EZ cells that had been transiently transfected with plasmids transcribing control RNA [lacZ; (−)] or PRE-RNA derived from the indicated Hox genes [leading strand:+; lagging strand: (−) (D,F)]. RT-PCR was used to detect the transcription of Hoxa5. XChIP detected the binding of EZH2 to the PRE-element of Hoxa5.

FIG. 16(A-C). Transcription of homeotic genes and their corresponding TRE- and PRE-elements in S2 cells and imaginal discs. (A) Photograph of ethidium bromide-stained agarose gel showing the reaction products of RT-PCR reactions detecting the transcripts of the indicated genes and their corresponding TRE- and PRE-elements. RNA was isolated from the indicated imaginal discs or the abdominal region of 3^{rd} instar larvae (AR). (B) RT-PCR reactions as in (A) detecting the transcription of Trr, Trx, E(Z) and Mdu target genes (upper panel) and their corresponding TRE- and PRE-elements (lower panel) in imaginal discs. (C) Photograph of ethidium bromide-stained agarose gel showing the reaction products of XChIP experiments detecting the interaction of Trr, Trx, E(Z) and Mdu with the TRE- and PRE-elements of the indicated target genes in S2 cells and imaginal discs.

FIG. 17. Transcription of homeotic genes and their corresponding TRE- and PRE-elements in MEF and MPMP cells. Photograph of ethidium bromide-stained agarose gel showing the reaction products of RT-PCR assays (PCR) and XChIP assays (X) using RNA and in vivo cross-linked chromatin isolated from MPMP and MEF cells. RT-PCR (PCR) monitored the transcription of Hox genes and the corresponding PRE-elements. XChIP monitored the interaction of M33 and DBSET1 with the PRE-elements.

FIG. 18(A-B). In vivo assay to detect RNA:protein interactions. (A) Schematic representation of the ‘yeast two hybrid screen’ designed to identify proteins binding to TRE- or PRE-transcripts. (B) Photographs of β-galactosidase activity assays in yeast colonies. Yeast cells were transformed with plasmids expressing the indicated RNA or fusion proteins. After 4 days colonies were transferred onto nitrocellulose incubated with X-Gal to detect β-galactosidase expression.

FIG. 19. Cell-type specific transcription pattern of HOXA5 RNAs. Photographs of RT-PCR assays detecting HOX TRE RNAs in the indicated cell types. RNA was isolated from primary myeloid cells and epithelial cells from breast, lung and stomach. PCR and RT-PCR detected the presence of HOX TRE-RNA in prepared RNA and cDNA pools, respectively.

FIG. 20(A-C). HOXA5 TRE RNA restores HOX5a and p53 expression and attenuates cancerous cell growth of breast cancer cells. (A) RT-PCR experiments detecting the transcription of HOXA5, the tumor suppressor gene p53 and tubulin in breast mammary epithelial cells and breast cancer epithelial cell (T47D). Note that p53 and HOXA5 transcription is significantly reduced in breast cancer cells. (B) Chromatin immunoprecipitation (XChIP) experiments detecting the interaction of HOXA5 with the promoter of HOXA5, p53, and tubulin in wild type and cancerous mammary epithelial cells. (C) Transient transcription of HOXA5 RNA restores HOXA5 and p53 transcription. Photographs of RT-PCR and XChIP experiments detecting HOXA5 and p53 transcription and the association of HOXA5 with the p53 promoter, respectively. Cancerous T47D cells were transfected with plasmids transcribing the HOXA4, HOXA5, or HOXA9 and green fluorescent protein (GFP). 60 h after transfection, transfected, GFP-expressing cells were isolated and used as a source for RNA and in vivo cross-linked chromatin. RT-PCR monitored the transcription of HOXA5 and p53. XChIP detected the association of HOXA5 with the p53 promoter.

FIG. 21(A-B). HOX TRE transcripts determine the developmental fate of embryonic stem cells. (A) The neuronal HOX RNA transcription pattern (see FIG. 19). (B) HOX RNAs establish neuronal cell fate in embryonic stem cells. Human embryonic stem cells were transfected with plasmids transcribing the indicated HOX TRE-RNAs. 64 h after transfection, RNA pools were isolated from transfected and untransfected (control) cells and reverse transcribed. PCR monitored the transcription of the neuronal marker gene neuroD in cDNA and genomic DNA pools.

### DETAILED DESCRIPTION

The present invention is based on the discovery that non-coding polynucleotides transcribed from chromosomal elements (CEs) associated with genes recruit epigenetic regulators to the corresponding CEs, and thereby mediate epigenetic regulation of gene transcription. The invention provides a method of regulating transcription of a gene that is a target for an epigenetic regulator, a method of characterizing the transcriptional activity of such a gene, a method of screening for a CE for an epigenetic regulator of a target gene, an isolated complex including an epigenetic regulator for a target gene, wherein the epigenetic regulator is specifically bound to a non-coding polynucleotide, and a method of screening for a modulator of transcription of a gene that is a target for an epigenetic regulator. The invention has implications for the characterization and control of genes affecting cell proliferation and differentiation.

### DEFINITIONS

Terms used in the claims and specification are defined as set forth below unless otherwise specified.

As used herein, the term “epigenetic regulator” refers to an agent that modulates gene expression by some means other than alteration of the nucleotide sequence of the target gene, such as, for example, by the posttranslational modification of histone proteins. The term “epigenetic regulator” encompasses full-length, wild-type proteins, as well as functional derivatives thereof, including fragments, such as, e.g., the SET-domain of histone methyltransferases.

An epigenetic regulator that acts to maintain a target gene in a transcriptionally active state is termed an “epigenetic activator” or a “transcriptional activator.”

An epigenetic regulator that acts to maintain a target gene in a transcriptionally inactive or silent state is termed an “epigenetic repressor” or a “transcriptional repressor.”

A “histone methyltransferase” is an enzyme that methylates one or more histone proteins.

A “SET-module” is a polypeptide segment that includes a SET-domain and flanking Cys-rich regions. The SET domain was first recognized as a conserved sequence in three D. melanogaster proteins: a modifier of position-effect variegation, Suppressor of variegation 3-9 (Su(var)3-9), the Polycomb-group chromatin regulator Enhancer of Zeste (E(z)), and the Trithorax-group chromatin regulator Trithorax (Trx). The domain, which is approximately 130 amino acids long, was characterized in 1998 (Jenuwein T., et al. (1998) SET domain proteins modulate chromatin domains in eu- and heterochromatin. Cell. Mol. Life. Sci. 54:80-93), and SET-domain proteins have now been found in all eukaryotic organisms studied. Seven main families of SET-domain proteins are known—the SUV39, SET1, SET2, EZ, RIZ, SMYD, and SUV4-20 families—as well as a few orphan members such as SET7/9 and SET8 (also called PR-SET7). Proteins within each family have similar sequence motifs surrounding the SET domain, and they often also share a higher level of similarity in the SET domain.

The phrase “transcriptional activity of a gene” encompasses any level of transcriptional activation ranging from no transcription to a maximal transcription level for a particular gene. Thus, “characterizing the transcriptional activity” of a gene includes an indication that the gene is not being transcribed, as well as an indication that the gene is being transcribed (i.e., a qualitative indication that the gene is “off” or “on”). In addition, this phrase encompasses quantitative indications that transcription is occurring at a higher or lower rate in a test sample than in a control sample.

A “cis-regulatory region of a gene” is a control region on the same chromosome as the gene that influences the transcriptional activity of the gene. Typical cis-regulatory regions include one or more binding sites for activators and/or repressors of the gene. In the case of epigenetic activators/repressors, the binding sites are termed “chromosomal elements (CEs)” for the epigenetic activators/repressors.

A CE is said to include a “sequence that is a template for a non-coding polynucleotide,” if the CE includes as sequence that is transcribed, but not translated into a polypeptide. The non-coding polynucleotide is said to “correspond to” the CE from which it is transcribed. The “non-coding polynucleotide” is typically “non-coding RNA.”

As used herein with respect to regulating gene transcription, a “modulator” is either an inhibitor or an enhancer of gene transcription.

The phrases “an effective amount” and “an amount sufficient to” refer to amounts of a biologically active agent that produce an intended biological activity.

A used herein, “a homeotic gene” refers to a gene that plays a role in development. Exemplary homeotic genes include “homeobox genes,” which play a role in bodily segmentation during embryonic development.

“Orthologs” are genes that are descended from a common ancestral gene and that share the same function.

As used herein, a “cancer cell” refers to any cell in which normal growth control is disrupted such that the cell displays abnormal growth characteristics, such as, e.g., growth in soft agar and/or immortal growth.

As used herein, a “stem cell” refers to a cell that can replicate indefinitely and that is omnipotent or pluripotent, i.e., the cell can differentiate into any or multiple other cell(s). Examples include self-regenerating cells in bone marrow, testes, embryos, and umbilical cords.

As used herein, a “dormant cell” refers to a cell that has stopped replicating.

As used herein, the term “biological sample” refers to any physiological medium containing a gene including a chromosomal element (CE) for an epigenetic regulator. A biological sample will generally also include the epigenetic regulator and a non-coding polynucleotide that specifically binds to the CE and is then specifically bound by the epigenetic regulator. A biological sample can be obtained, for example, from cell culture or directly from an organism and may be subjected to any desired processing steps, e.g., concentration or dilution.

The following terms encompass polypeptides that are identified in Genbank by the following designations, as well as polypeptides that are at least about 70% identical to polypeptides identified in Genbank by these designations: Ultrabithorax (Ubx), abdominal B (abd-B), wingless (wg), Sex-combs reduced (SCR), Antennapedia (ANTP), any Hox gene, Trithorax (Trx), Trithorax-related (Trr), absent small and homeotic discs (Ash1), human Trx, human Ash1, human Ash2, Mixed Lineage Leukemia (MLL), MLL-related (MLL-1, MLL-2, MLL-3, MLL-4, MLL-5), ALL-1, ALL-2, ALL-3, ALL-4, ALL-5, D. melanogaster Enhancer of Zeste (E(Z)), Polycomb (PC), Medusa (Mdu), Su(var)3-5, Su(var)3-7, Su(var)3-9, Su(var)3-6, Su(var)2-1, Su(var)2-10, Su(var)3-3, mammalian Enhancer of Zeste (EZH2), M33, SETDB1, ENX-2, mammalian SUV39H1, SUV39H2. In alternative embodiments, these terms encompass polypeptides identified in Genbank by these designations and polypeptides sharing at least about 80, 90, 95, 96, 97, 98, or 99% identity.

As used with respect to polypeptides, polynucleotides, or complexes, the terms “isolated” and “purified” are used interchangeably and refer to a polypeptide, polynucleotide, or complex that has been separated from at least one other component that is typically present with the polypeptide or polynucleotide. Thus, a naturally occurring polypeptide is isolated if it has been purified away from at least one other component that occurs naturally with the polypeptide or polynucleotide. A recombinant polypeptide or polynucleotide is isolated if it has been purified away from at least one other component present when the polypeptide or polynucleotide is produced.

The terms “polypeptide” and “protein” are used interchangeably herein to refer a polymer of amino acids, and unless otherwise specified, include atypical amino acids that can function in a similar manner to naturally occurring amino acids.

The terms “amino acid” or “amino acid residue,” include naturally occurring L-amino acids or residues, unless otherwise specifically indicated. The commonly used one- and three-letter abbreviations for amino acids are used herein (Lehninger, A. L. (1975) Biochemistry, 2d ed., pp. 71-92, Worth Publishers, N.Y.). The terms “amino acid” and “amino acid residue” include D-amino acids as well as chemically modified amino acids, such as amino acid analogs, naturally occurring amino acids that are not usually incorporated into proteins, and chemically synthesized compounds having the characteristic properties of amino acids (collectively, “atypical” amino acids). For example, analogs or mimetics of phenylalanine or proline, which allow the same conformational restriction of the peptide compounds as natural Phe or Pro are included within the definition of “amino acid.”

Exemplary atypical amino acids, include, for example, those described in International Publication No. WO 90/01940, as well as 2-amino adipic acid (Aad) which can be substituted for Glu and Asp; 2-aminopimelic acid (Apm), for Glu and Asp; 2-aminobutyric acid (Abu), for Met, Leu, and other aliphatic amino acids; 2-aminoheptanoic acid (Ahe), for Met, Leu, and other aliphatic amino acids; 2-aminoisobutyric acid (Aib), for Gly; cyclohexylalanine (Cha), for Val, Leu, and Ile; homoarginine (Har), for Arg and Lys; 2,3-diaminopropionic acid (Dpr), for Lys, Arg, and His; N-ethylglycine (EtGly) for Gly, Pro, and Ala; N-ethylasparagine (EtAsn), for Asn and Gln; hydroxyllysine (Hyl), for Lys; allohydroxyllysine (Ahyl), for Lys; 3-(and 4-) hydroxyproline (3Hyp, 4Hyp), for Pro, Ser, and Thr; allo-isoleucine (Aile), for Ile, Leu, and Val; amidinophenylalanine, for Ala; N-methylglycine (MeGly, sarcosine), for Gly, Pro, and Ala; N-methylisoleucine (MeIle), for Ile; norvaline (Nva), for Met and other aliphatic amino acids; norleucine (Nle), for Met and other aliphatic amino acids; ornithine (Orn), for Lys, Arg, and His; citrulline (Cit) and methionine sulfoxide (MSO) for Thr, Asn, and Gln; N-methylphenylalanine (MePhe), trimethylphenylalanine, halo (F, Cl, Br, and I) phenylalanine, and trifluorylphenylalanine, for Phe.

As used with reference to a polypeptide, the term “full-length” refers to a polypeptide having the same length as the mature wild-type polypeptide.

The term “fragment” is used herein with reference to a polypeptide or a nucleic acid molecule to describe a portion of a larger molecule. Thus, a polypeptide fragment can lack an N-terminal portion of the larger molecule, a C-terminal portion, or both. Polypeptide fragments are also referred to herein as “peptides.” A fragment of a nucleic acid molecule can lack a 5′ portion of the larger molecule, a 3′ portion, or both. Nucleic acid fragments are also referred to herein as “oligonucleotides.” Oligonucleotides are relatively short nucleic acid molecules, generally shorter than 200 nucleotides, more particularly, shorter than 100 nucleotides, most particularly, shorter than 50 nucleotides. Typically, oligonucleotides are single-stranded DNA molecules.

A “subsequence” of an amino acid or nucleotide sequence is a portion of a larger sequence.

The terms “identical” or “percent identity,” in the context of two or more amino acid or nucleotide sequences, refer to two or more sequences or subsequences that are the same or have a specified percentage of amino acid residues or nucleotides that are the same, when compared and aligned for maximum correspondence, as measured using a sequence comparison algorithm or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman (1988) Proc. Natl. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra).

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle (1987) J. Mol. Evol. 35:351-360. The method used is similar to the method described by Higgins & Sharp (1989) CABIOS 5: 151-153. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of algorithm that is suitable for determining percent sequence identity and sequence similarity is the BLAST algorithm, which is described in Altschul et al. (1990) J. Mol. Biol. 215: 403-410. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul (1993) Proc. Natl. Acad. Sci. USA, 90: 5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

As used with reference to polypeptides, the term “wild-type” refers to any polypeptide having an amino acid sequence present in a polypeptide from a naturally occurring organism, regardless of the source of the molecule; i.e., the term “wild-type” refers to sequence characteristics, regardless of whether the molecule is purified from a natural source; expressed recombinantly, followed by purification; or synthesized.

The term “amino acid sequence variant” refers to a polypeptide having an amino acid sequence that differs from a wild-type amino acid sequence by the addition, deletion, or substitution of an amino acid.

As used with reference to a polypeptide or polypeptide fragment, the term “derivative” includes amino acid sequence variants as well as any other molecule that differs from a wild-type amino acid sequence by the addition, deletion, or substitution of one or more chemical groups. “Derivatives” retain at least one biological or immunological property of a wild-type polypeptide or polypeptide fragment, such as, for example, the biological property of specific binding to a receptor and the immunological property of specific binding to an antibody.

A “signal sequence” is an amino acid sequence that directs the secretion of a polypeptide fused to the signal sequence. As used in recombinant expression, the polypeptide is secreted from a cell expressing the polypeptide into the culture medium for ease of purification.

An “epitope tag” is an amino acid sequence that defines an epitope for an antibody. Epitope tags can be engineered into polypeptides or peptides of interest to facilitate purification or detection. Exemplary epitope tags include the green fluorescent protein (GFP), hemagglutinin, and FLAG epitope tags.

The term “polynucleotide” refers to a deoxyribonucleotide or ribonucleotide polymer, and unless otherwise specified, includes known analogs of natural nucleotides that can function in a similar manner to naturally occurring nucleotides.

The term “polynucleotide” refers any form of DNA or RNA, including, for example, genomic DNA; complementary DNA (cDNA), which is a DNA representation of mRNA, usually obtained by reverse transcription of messenger RNA (mRNA) or amplification; DNA molecules produced synthetically or by amplification; and mRNA.

The term “polynucleotide” encompasses double-stranded polynucleotides, as well as single-stranded molecules. Double-stranded polynucleotides that encode a protein contain a “sense” polynucleotide strand hydrogen-bonded to an “antisense” polynucleotide strand. The sense polynucleotide strand is the strand whose nucleotide sequence, when translated, provides the amino acid sequence of the encoded protein. In double-stranded polynucleotides, the polynucleotide strands need not be coextensive (i.e, a double-stranded polynucleotide need not be double-stranded along the entire length of both strands).

As used herein, the term “complementary” refers to the capacity for precise pairing between two nucleotides. I.e., if a nucleotide at a given position of a nucleic acid molecule is capable of hydrogen bonding with a nucleotide of another nucleic acid molecule, then the two nucleic acid molecules are considered to be complementary to one another at that position.

The term “vector” is used herein to describe a construct, typically a DNA construct, containing a polynucleotide. Such a vector can be propagated stably or transiently in a host cell. The vector can, for example, be a plasmid, a viral vector, a cosmid, a BAC, a YAC, or simply a potential genomic insert. Once introduced into a suitable host, the vector may replicate and function independently of the host genome, or may, in some instances, integrate into the host genome.

“Expression vector” refers to a construct containing a polynucleotide molecule that is operably linked to a control sequence capable of effecting the expression of the polynucleotide in a suitable host. Exemplary control sequences include a promoter to effect transcription, an optional operator sequence to control transcription, a sequence encoding a suitable mRNA ribosome binding site, and sequences that control termination of transcription and translation.

As used herein, the term “operably linked” refers to a functional linkage between two sequences, such a control sequence (typically a promoter) and the linked sequence.

The term “host cell” refers to a cell capable of maintaining a vector either transiently or stably. Host cells of the invention include, but are not limited to, bacterial cells, yeast cells, insect cells, plant cells, and mammalian cells. Other host cells known in the art, or which become known, are also suitable for use in the invention.

As used herein, an “antibody” refers to a protein consisting of one or more polypeptides substantially encoded by immunoglobulin genes or fragments of immunoglobulin genes. The recognized immunoglobulin genes include the kappa, lambda, alpha, gamma, delta, epsilon, and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD, and IgE, respectively.

A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region thereby converting the (Fab′)₂ dimer into an Fab′ monomer. The Fab′ monomer is essentially an Fab with part of the hinge region (See, Fundamental Immunology, W. E. Paul, ed., Raven Press, N.Y. (1993), for a more detailed description of other antibody fragments). While various antibody fragments are defined in terms of the digestion of an intact antibody, one of skill will appreciate that such Fab′ fragments may be synthesized de novo either chemically or by utilizing recombinant DNA methodology. Thus, the term “antibody”, as used herein also includes antibody fragments either produced by the modification of whole antibodies or synthesized de novo using recombinant DNA methodologies. Preferred antibodies include single chain antibodies, more preferably single chain Fv (scFv) antibodies in which a variable heavy and a variable light chain are joined together (directly or through a peptide linker) to form a continuous polypeptide.

An “antigen-binding site” or “binding portion” refers to the part of an immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions” or “FRs.” Thus, the term “FR” refers to amino acid sequences that are naturally found between and adjacent to hypervariable regions in immunoglobulins. In an antibody molecule, the three hypervariable regions of a light chain and the three hypervariable regions of a heavy chain are disposed relative to each other in three dimensional space to form an antigen binding “surface.” This surface mediates recognition and binding of the target antigen. The three hypervariable regions of each of the heavy and light chains are referred to as “complementarity determining regions” or “CDRs” and are characterized, for example by Kabat et al. Sequences of proteins of immunological interest, 4th ed. U.S. Dept. Health and Human Services, Public Health Services, Bethesda, Md. (1987).

A single chain Fv (“scFv”) antibody is a covalently linked V_{H}::V_{L} heterodimer that forms a single antigen binding domain. Two scFv chains can be linked, covalently or noncovalently, to form an (scFv′)₂ antibody, which has two antigen binding domains, which can be the same or different.

As used herein, the term “antibody” includes any antibody conjugated to any other substance, e.g., labeled antibodies, antibodies conjugated to polymeric beads, etc.

As used herein, the terms “antibody binding” and “immunoreactivity” refer to the non-covalent interactions of the type which occur between an immunoglobulin molecule and an antigen for which the immunoglobulin is specific. The strength or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and on geometric parameters that equally influence the rate in both directions. Thus, both the “on rate constant” (Kₒₙ) and the “off rate constant” (K_{off}) can be determined by calculation of the concentrations and the actual rates of association and dissociation. The ratio of K_{off}/Kₒₙ enables cancellation of all parameters not related to affinity and is thus equal to the dissociation constant K_{d}. See, generally, Davies et al. Ann. Rev. Biochem., 59: 439-473 (1990).

The phrase “specifically binds” is used herein with reference to a polypeptide or polynucleotide to describe a high-affinity binding reaction characterized by the interaction of particular binding domains on each molecule, as opposed, for example, to non-specific “sticking.” In the case of polynucleotides, specific binding is typically achieved by hybridization of complementary nucleotide sequences.

The term “in vivo cross-linked chromatin immunoprecipitation” refers to a technique whereby chromatin is cross-linked, generally by treating cells or tissue with a chemical agent (e.g., formamide) to preserve the association of protein and DNA in the chromatin structure. The resulting complex is typically sheared, followed by immunoprecipitation using an antibody specific for one of the proteins in the complex. Immunoprecipitation results in recovery of the DNA, and, if applicable, RNA, with which the protein of interest is complexed. This DNA (and any RNA present) can subsequently be analyzed (e.g., by polymerase chain reaction (PCR)) to identify the nucleotide sequence(s) associated with the protein of interest.

A “test agent” is any agent that can be screened in the prescreening or screening assays of the invention. The test agent can be any suitable composition, including a small molecule, peptide, polypeptide, oligonucleotide, or polynucleotide.

### ABBREVIATIONS

abd-B—Drosophila abdominal B gene (homeotic gene)

Ash1—Drosophila “absent small and homeotic discs” gene (epigenetic activator)

ANTP—Drosophila Antennapedia gene (homeotic gene)

CE—chromosomal element

E(Z)—Drosophila “Enhancer of Zeste” gene (epigenetic repressor)

EZH2—mammalian “Enhancer of Zeste” gene (epigenetic repressor)

M33—mammalian M33 gene (epigenetic repressor)

Mdu—Drosophila Medusa gene (epigenetic repressor)

MLL—mammalian “Mixed Lineage Leukemia” gene (epigenetic activator)

NChIP—native chromatin immunoprecipitation

ncRNA—non-coding RNA

PC—Drosophila Polycomb gene (epigenetic repressor)

SCR—Drosophila Sex-combs reduced gene (homeotic gene)

SETDB1—mammalian SETDB1 gene (epigenetic repressor)

Trr—Drosophila Trithorax-related gene (epigenetic activator)

Trx—Drosophila Trithorax (epigenetic activator)

Ubx—Drosophia Ultrabithorax gene (homeotic gene)

wg—Drosophila wingless gene (homeotic gene)

XchIP—in vivo cross-linked chromatin immunoprecipitation

I. Regulation of Transcription of Genes that are Targets for Epigenetic Regulators

A. In General

The invention provides a method of regulating transcription of a gene that is a target for an epigenetic regulator. The method is applicable to any target gene that has a cis-regulatory region including a chromosomal element (CE) for the epigenetic regulator, wherein the CE comprises a sequence that is a template for a non-coding polynucleotide. The non-coding polynucleotide recruits the epigenetic regulator to the CE, which either activates or represses transcription of the target gene.

The method entails contacting cells comprising the gene and the epigenetic regulator with an effective amount of a modulator. The modulator alters the level of: (1) the non-coding polynucleotide; (2) the specific binding of the non-coding polynucleotide to the target gene; and/or (3) the specific binding of the epigenetic regulator to the non-coding polynucleotide.

In one embodiment, the modulator reduces one or more of the above levels. In this embodiment, if the epigenetic regulator is a transcriptional activator, the modulator represses transcription of the target gene. Alternatively, if the epigenetic regulator is a transcriptional repressor, the modulator activates transcription of the target gene.

In another embodiment, the modulator increases one of more of these levels. In this case, if the epigenetic regulator is a transcriptional activator, the modulator activates transcription of the target gene. Alternatively, if the epigenetic regulator is a transcriptional repressor, the modulator represses transcription of the target gene.

B. Target Gene

In preferred embodiments, the target gene is a homeotic gene. Homeotic genes generally regulate the expression of other genes, acting as “master switches” in development. Exemplary homeotic genes include “homeobox genes.” The discovery of the homeobox as a conserved DNA sequence element in several Drosophila genes responsible for controlling the identity of body segments prompted searches for related genes in other organisms. Homeoboxes have since been discovered in the genome of all metazoan organisms, and several hundred unique homeobox genes have been defined in mice and humans (Gehring, W. J. et al., Annu. Rev. Biochem. 63:487-526 (1994); Stein, S. et al., Mech. Develop. 55:91-108 (1996)). The homeobox encodes a 60-amino acid domain, termed the homeodomain, that includes a helix-turn-helix motif recognized to be structurally-related to the DNA binding domain of several procaryotic proteins and to the products of the yeast mating type locus (Laughon, A. and Scott, M. P., Nature 310:25-31 (1984); Shepherd, J. C. W. et al., Nature 310:70-71 (1984)). NMR and crystallographic analyses have confirmed that the homeodomain binds DNA (Kissinger, C. R. et al., Cell 63:579-590 (1990); Otting, G. et al., EMBO J. 9:3085-3092 (1990)). As predicted by the nature of the phenotypes produced when these genes are mutated, both biochemical and genetic analyses have established that the products of homeobox genes are transcriptional regulatory molecules (McGinnis, W. and Krumlauf, R., Cell 68:283-302 (1992)).

The predicted amino acid sequence of the known homeodomains serves as the principal identifier that allows them to be classified into a minimum of 20 distinct groups (Gehring, W. J. et al., Annu. Rev. Biochem. 63:487-526 (1994); Stein, S. et al., Mech. Develop. 55:91-108 (1996)).

The majority of studies aimed at characterizing the functions of homeobox genes have focused principally on their developmental roles (McGinnis, W. and Krumlauf, R., Cell 68:283-302 91992); Krumlauf, R., Cell 78:191-201 (1994)). A prominent example is the Hox family of genes, whose members have been demonstrated to play critical roles in pattern formation during embryogenesis along the anteroposterior body axis of divergent species (Krumlauf, R., Cell 78:191-201 (1994)). Some of the Hox genes, as well as members of other classes of homeobox genes, are also expressed during organogenesis, and a few of these have been reported to be expressed in adult tissues.

In specific embodiments, the homeotic gene can be, for example, any of the Drosophila genes Ultrabithorax (Ubx), abdominal B (abd-B), wingless (wg), Sex-combs reduced (SCR), and Antennapedia (ANTP), or an ortholog thereof, particularly a vertebrate ortholog, preferably a mammalian ortholog, and more preferably a human ortholog. Additional examples of homeotic genes useful in the invention includes those shown in Tables A and B, below.

C. Epizenetic Regulator

The epigenetic regulator either activates or represses transcription of the target gene, and this action requires a non-coding polynucleotide that recruits the epigenetic regulator to a CE for the target gene. The method of the invention is applicable to any epigenetic regulator that functions in this manner. Exemplary regulators of this type include epigenetic regulators that mediate the posttranslational modification of histones (H1, H2A, H₂B, H3, H4). Several epigenetic activators (Trx, Trr, Ash1) and repressors [E(Z)] are lysine-specific histone methyltransferases (HMTs) that contain an enzymatic module (SET-module) consisting of the SET-domain and flanking cysteine-rich regions. Additional examples of epigenetic regulators having SET-domains are given in Tables C and D, below. Methylation of lysine residues in H3 and H4 has been correlated with epigenetic activation and repression (6). One hallmark of epigenetic repression is the methylation of lysines 9 (H3-K9) and 27 (H3-K27) in histone H3 (7,8). In contrast, epigenetic activation has been linked to methylation of lysine 4 in H3 (H3-K4) (4,6).

Accordingly, in particular embodiments of the method of the invention, the epigenetic regulator is one that activates transcription of the target gene. Examples of epigenetic activators useful in conjunction with the method include Trithorax (Trx), Trithorax-related (Trr), absent small and homeotic discs (Ash1), human Trx, human Ash1, human Ash2, Mixed Lineage Leukemia (MLL), MLL-related (MLL-1, MLL-2, MLL-3, MLL-4, MLL-5), ALL-1, ALL-2, ALL-3, ALL-4, ALL-5, and orthologs thereof.

In other embodiments, the epigenetic regulator is one that represses transcription of the target gene. Examples of epigenetic repressors useful in conjunction with the method of the invention include D. melanogaster Enhancer of Zeste (E(Z)), Polycomb (PC), Medusa (Mdu), Su(var)3-5, Su(var)3-7, Su(var)3-9, Su(var)3-6, Su(var)2-1, Su(var)2-10, Su(var)3-3, mammalian Enhancer of Zeste (EZH2), M33, SETDB1, ENX-2, mammalian SUV39H1, SUV39H2, and orthologs thereof.

Orthologs of epigenetic regulators useful in the method can be from any multicellular organism, particularly vertebrates, preferably mammals, and more preferably humans.

D. Non-coding Polynucleotide

The method of the invention is applicable to any system including a non-coding polynucleotide that functions as described above. Generally, naturally occurring non-coding polynucleotides are RNA. However, non-coding polynucleotides useful in the invention include deoxyribonucleotide, as well as ribonucleotide, polymers, and known analogs of natural nucleotides that can function in a similar manner to naturally occurring nucleotides.

In nature, the non-coding polynucleotide has a sequence that is (1) capable of specifically binding the CE from which it is transcribed and (2) capable of specifically binding the appropriate epigenetic regulator. As described in greater detail below, either or both of these binding activities can be disrupted in those embodiments in which it is desirable to inhibit the normal functioning of the non-coding polynucleotide.

E. Cells

Transcription can be regulated according to this method in any cell that contains a suitable target gene and epigenetic regulator. The cell may be one that transcribes the relevant non-coding polynucleotide, depending on the particular application. For example, if the epigenetic regulator is one that activates transcription of the target gene, and the method is carried out to repress this transcription, the cell employed will transcribe the non-coding polynucleotide, and the modulator will be administered to reduce the level of the non-coding polynucleotide or to inhibit its binding to the target gene or the epigenetic regulator. Conversely, if the method is carried out to activate this transcription, cells that do not transcribe the non-coding polynucleotide can be employed. In this instance, the modulator will be one that provides the non-coding polynucleotide to the cell, such that the non-coding polynucleotide can recruit the epigenetic regulator to the target gene, thereby activating its transcription. As those skilled in the art readily appreciate, the method can also be carried out to enhance transcription of the target gene in cells that transcribe the non-coding polynucleotide.

Cells useful in the method can be from any multicellular animal, including invertebrates, such as insects, and vertebrates. Cells from any of vertebrate can be employed, particularly mammals, such as dogs, cats, sheep, cattle, pigs, and rodents (such as mice, rats, hamsters, and guinea pigs); and more particularly primates, such as humans, chimpanzees, gorillas, macaques, and baboons.

The method can be carried out on cells in vivo or in vitro. Suitable in vitro applications include, for example, the use of cultured cells or cells in a biological sample (e.g., whole blood, plasma, serum, saliva, synovial fluid, cerebrospinal fluid, bronchial lavage, ascites fluid, bone marrow aspirate, pleural effusion, urine, or tissue, cells, or fractions thereof) derived from an animal.

F. Modulation of the Level of the Non-Coding Polynucleotide

In one embodiment of the method, transcription of the target gene is modulated by altering the level of the non-coding polynucleotide.

1. Increasing the Level of the Non-Coding Polynucleotide

Increasing the level of the non-coding polynucleotide in a cell enhances the function of the corresponding epigenetic regulator by recruiting more regulator to the CE of the target gene, thereby enhancing regulation of transcription. The level of non-coding polynucleotide can be increased from a baseline level or, alternatively, non-coding polynucleotide can be provided to a cell in which it is not present, thereby activating a previously silent target gene or repressing a previously active target gene (depending upon whether the epigenetic regulator activates or represses transcription, respectively).

The level of non-coding polynucleotide can be increased by any convenient method for providing a polynucleotide to a cell. In general, a polynucleotide can be produced outside of the cell and then introduced into the target cell or, alternatively, the polynucleotide can be produced inside of the target cell using a vector.

a. Synthesis and Administration of Non-Coding Polynucleotides

Non-coding polynucleotides produced outside of the target cell can be produced synthetically using standard techniques. Oligonucleotides are conveniently synthesized, for example, by the well-known phosphotriester and phosphodiester methods, especially the automated versions thereof. A standard automated method uses diethylphosphoramidites as starting materials, which can be purchased commercially or synthesized as described by Beaucage et al., Tetrahedron Letters 22: 1859-1962 (1981) or in U.S. Pat. No. 4,458,066. Equipment for such synthesis is sold by several vendors (e.g. Applied Biosystems). Long sequences can be produced, if desired, by designing and synthesizing suitable oligonucleotides that can be linked together, e.g., using standard ligation reactions.

In the case of synthetic polynucleotides, it may be advantageous to stabilize the polynucleotides described herein or to produce polynucleotides that are modified to better adapt them for particular applications. To this end, the polynucleotides of the invention can contain phosphorothioates, phosphotriesters, methyl phosphonates, short chain alkyl or cycloalkyl intersugar linkages or short chain heteroatomic or heterocyclic intersugar (“backbone”) linkages. Most preferred are phosphorothioates and those with CH2-NH—O—CH2, CH2-N(CH3)-O—CH2 (known as the methylene(methylimino) or MMI backbone) and CH2-O—N(CH3)-CH2, CH2-N(CH3)-N(CH3)-CH2, and 0-N(CH3)-CH2-CH backbones (where phosphodiester is O—P—O—CH2). Also preferred are polynucleotides having morpholino backbone structures. Summerton, J. E. and Weller, D. D., U.S. Pat. No. 5,034,506. Other preferred embodiments use a protein-nucleic acid or peptide-nucleic acid (PNA) backbone, wherein the phosphodiester backbone of the polynucleotide is replaced with a polyamide backbone, the bases being bound directly or indirectly to the aza nitrogen atoms of the polyamide backbone. P. E. Nielsen, M. Egholm, R. H. Berg, O. Buchardt, Science 1991, 254, 1497. Polynucleotides of the invention can contain alkyl and halogen-substituted sugar moieties and/or can have sugar mimetics such as cyclobutyls in place of the pentofuranosyl group. In other preferred embodiments, the polynucleotides can include at least one modified base form or “universal base” such as inosine. Polynucleotides can, if desired, include an RNA cleaving group, a cholesteryl group, a reporter group, an intercalator, a group for improving the pharmacokinetic properties of the polynucleotide, and/or a group for improving the pharmacodynamic properties of the polynucleotide.

Non-coding polynucleotides intended for administration to cells can be formulated into compositions including other components, such as for example, a storage solution, such as a suitable buffer, e.g., a physiological buffer. Preferably, such compositions also include a component that facilitates entry of the polynucleotide into a cell. Components that facilitate intracellular delivery of polynucleotides are well-known and include, for example, lipids, liposomes, water-oil emulsions, polyethylene imines and dendrimers, any of which can be used in compositions according to the invention. Lipids are among the most widely used components of this type, and any of the available lipids or lipid formulations can be employed with polynucleotides useful in the invention. Typically, cationic lipids are preferred. Preferred cationic lipids include N-[1-(2,3-dioleyloxy)propyl]-n,n,n-trimethylammonium chloride (DOTMA), dioleoyl phosphotidylethanolamine (DOPE), and/or dioleoyl phosphatidylcholine (DOPC).

In another embodiment, non-coding polynucleotides are complexed to dendrimers, which can be used to introduce the polynucleotides into cells. Dendrimer polycations are three-dimensional, highly ordered oligomeric and/or polymeric compounds typically formed on a core molecule or designated initiator by reiterative reaction sequences adding the oligomers and/or polymers and providing an outer surface that is positively changed. Suitable dendrimers include, but are not limited to, “starburst” dendrimers and various dendrimer polycations. Methods for the preparation and use of dendrimers to introduce polynucleotides into cells in vivo are well known to those of skill in the art and described in detail, for example, in PCT/US83/02052 and U.S. Pat. Nos. 4,507,466; 4,558,120; 4,568,737; 4,587,329; 4,631,337; 4,694,064; 4,713,975; 4,737,550; 4,871,779; 4,857,599; and 5,661,025.

A wide variety of techniques are available for introducing polynucleotides into cells, and a suitable technique for a particular application can readily be determined by those of skill in the art. Some of these are discussed below in conjunction with a preferred means of increasing the level of non-coding polynucleotide, which entails the use of a vector to transcribe the non-coding polynucleotide in the cell.

For therapeutic use, polynucleotides useful in the invention are formulated in a manner appropriate for the particular indication. U.S. Pat. No. 6,001,651 to Bennett et al. describes a number of pharmaceutically acceptable compositions and formulations suitable for use with an oligonucleotide therapeutic as well as methods of administering such oligonucleotides.

b. Transcription of Non-Coding Polynucleotides

A non-coding polynucleotide of the invention can be incorporated into a vector for propagation and/or transcription in a host cell or in a cell-free reaction mixture. Such vectors typically contain a replication sequence capable of effecting replication of the vector in a suitable host cell (i.e., an origin of replication) as well as sequences encoding a selectable marker, such as an antibiotic resistance gene. Upon transformation of a suitable host, the vector can replicate and function independently of the host genome or integrate into the host genome. Vector design depends, among other things, on the intended use and host cell for the vector, and the design of a vector of the invention for a particular use and host cell is within the level of skill in the art.

If the vector is intended for transcription of a sequence contained therein, the vector includes one or more control sequences capable of effecting and/or enhancing the transcription of the operably linked sequence. The inclusion in a vector of a gene complementing an auxotrophic deficiency in the chosen host cell allows for the selection of host cells transformed with the vector. A vector according to the invention can also include other sequences, such as, for example, a nucleic acid sequence encoding an amplifiable gene.

In preferred embodiments, the vector is a transcription vector, which includes an RNA promoter sequence useful for transcribing an operably linked sequence into non-coding RNA. Suitable RNA promoter sequences are capable of binding an RNA polymerase and contain a transcriptional start site. The promotor sequence usually includes between about 15 and about 250 nucleotides, preferably between about 25 and about 60 nucleotides, from a naturally occurring RNA polymerase promoter, a consensus promoter sequence (Alberts et al., in Molecular Biology of the Cell, 2d Ed., Garland, N.Y. (1989), or a modified version thereof. The promoter sequence employed in a particular vector is generally one recognized by an RNA polymerase present in the cell in which transcription of non-coding RNA is desired. Alternatively, a gene encoding the required polymerase can be introduced into the cell as part of the transcription vector or in a separate vector.

A wide variety of promoters and polymerases showing specificity for their cognate promoter are known, including phage or viral promoters, prokaryotic promoters, and eukaryotic promoters. Examples include the T3, T7, and SP6 phage promoter/polymerase systems. Probably the best studied is E. coli phage T7. T7 makes an entirely new polymerase that is highly specific for the 17 late T7 promoters. Rather than having two separate highly conserved regions like E. coli promoters, the late T7 promoters have a single highly conserved sequence from −17 to +6, relative to the RNA start site. The Salmonella phage SP6 is very similar to T7. Although most RNA polymerases recognize double-stranded promoters, E. coli phage N4 makes an RNA polymerase that recognizes early N4 promoters on native single stranded N4 DNA. A detailed description of promoters and RNA synthesis upon DNA templates is found in Watson et al., Molecular Biology of The Gene, 4th Ed., Chapters 13-15, Benjamin/Cummings Publishing Co., Menlo Park, Calif.

The RNA promoter sequence is linked to the sequence to facilitate transcription in the presence of ribonucleotides and an RNA polymerase under suitable conditions. The RNA promoter is upstream (5′) of the sequence in an orientation that permits transcription to yield non-coding RNA that is capable of specifically binding its corresponding CE and epigenetic regulator. Any type of linkage that meets this criterion can be employed, however nucleotide linkages are preferred. A linker oligonucleotide between the components, if present, typically includes between about 5 and about 20 bases, but may be smaller or larger as desired.

A vector of the present invention is produced by linking desired elements by ligation at convenient restriction sites. If such sites do not exist, suitable sites can be introduced by standard mutagenesis (e.g., site-directed or cassette mutagenesis) or synthetic oligonucleotide adaptors or linkers can be used in accordance with conventional practice.

Viral vectors are of particular interest for use in delivering non-coding polynucleotides of the invention to a cell or organism. Widely used vector systems include, but are not limited to adenovirus, adeno associated virus, and various retroviral expression systems. The use of adenoviral vectors is well known to those of skill and is described in detail, e.g., in WO 96/25507. Exemplary adenoviral vectors are described by Wills et al. (1994) Hum. Gene Therap. 5: 1079-1088. Adenoviral vectors suitable for use in the invention are also commercially available. For example, the Adeno-X™ Tet-Off™ gene expression system, sold by Clontech, provides an efficient means of introducing inducible heterologous sequences into most mammalian cells.

Adeno-associated virus (AAV)-based vectors used to transduce cells with polynucleotides, e.g., in the in vitro production of polynucleotides and peptides, and in vivo and ex vivo gene therapy procedures are described, for example, by West et al. (1987) Virology 160:38-47; Carter et al. (1989) U.S. Pat. No. 4,797,368; Carter et al. WO 93/24641 (1993); Kotin (1994) Human Gene Therapy 5:793-801; Muzyczka (1994) J. Clin. Invst. 94:1351 for an overview of AAV vectors. Lebkowski, U.S. Pat. No. 5,173,414; Tratschin et al. (1985) Mol. Cell. Biol. 5(11):3251-3260; Tratschin, et al. (1984) Mol. Cell. Biol., 4: 2072-2081; Hermonat and Muzyczka (1984) Proc. Natl. Acad. Sci. USA, 81: 6466-6470; McLaughlin et al. (1988) and Samulski et al. (1989) J. Virol., 63:03822-3828. Cell lines that can be transformed by rAAV include those described in Lebkowski et al. (1988) Mol. Cell. Biol., 8:3988-3996.

Widely used retroviral vectors include those based upon murine leukemia virus (MuLV), gibbon ape leukemia virus (GaLV), Simian Immunodeficiency virus (SIV), human immunodeficiency virus (HIV), alphavirus, and combinations thereof (see, e.g., Buchscher et al. (1992) J. Virol. 66(5) 2731-2739; Johann et al. (1992) J. Virol. 66 (5):1635-1640 (1992); Sommerfelt et al., (1990) Virol. 176:58-59; Wilson et al. (1989) J. Virol. 63:2374-2378; Miller et al., J. Virol. 65:2220-2224 (1991; Wong-Staal et al., PCT/US94/05700, and Rosenburg and Fauci (1993) in Fundamental Immunology, Third Edition Paul (ed) Raven Press, Ltd., New York and the references therein, and Yu et al. (1994) Gene Therapy, supra; U.S. Pat. No. 6,008,535, and the like). Other suitable viral vectors include those derived from herpes virus, lentivirus, and vaccinia virus.

A vector of the present invention is introduced into a host cell by any convenient method, which will vary depending on the vector-host system employed. Generally, a vector is introduced into a host cell by transformation (also known as “transfection”) or infection with a virus (e.g., phage) bearing the vector. If the host cell is a prokaryotic cell (or other cell having a cell wall), convenient transformation methods include the calcium treatment method described by Cohen, et al. (1972) Proc. Natl. Acad. Sci., USA, 69:2110-14. If a prokaryotic cell is used as the host and the vector is a phagemid vector, the vector can be introduced into the host cell by infection. Yeast cells can be transformed using polyethylene glycol, for example, as taught by Hinnen (1978) Proc. Natl. Acad. Sci, USA, 75:1929-33. Mammalian cells are conveniently transformed using the calcium phosphate precipitation method described by Graham, et al. (1978) Virology, 52:546 and by Gorman, et al. (1990) DNA and Prot. Eng. Tech., 2:3-10. However, other known methods for introducing DNA into host cells, such as nuclear injection, electroporation, and protoplast fusion also are acceptable for use in the invention.

2. Decreasing the Level of the Non-Coding Polynucleotide

Decreasing the level of the non-coding polynucleotide in a cell inhibits the function of the corresponding epigenetic regulator because there is less non-coding polynucleotide available to recruit the regulator to the CE of the target gene. If the epigenetic regulator activates transcription, decreasing the level of non-coding polynucleotide will oppose this effect, inhibiting transcription of the target gene. Conversely, if the epigenetic regulator represses transcription, decreasing the level of non-coding polynucleotide with activate or stimulate transcription.

a. Catalytic RNAs and DNAs

### (1) Ribozymes

In one approach, the level of non-coding polynucleotides can be reduced using ribozymes. As used herein, “ribozymes” include RNA molecules that contain antisense sequences for specific recognition, and an RNA-cleaving enzymatic activity. The catalytic strand cleaves a specific site in a target non-coding polynucleotide sequence, preferably at greater than stoichiometric concentration. The ribozymes of the invention typically consist of RNA, but such ribozymes may also be composed of polynucleotide molecules comprising chimeric polynucleotide sequences (such as DNA/RNA sequences) and/or polynucleotide analogs (e.g., phosphorothioates).

Ribozymes useful in the method may, e.g., be in the form of a “hammerhead” (for example, as described by Forster and Symons (1987) Cell 48: 211-220; Haseloff and Gerlach (1988) Nature 328: 596-600; Walbot and Bruening (1988) Nature 334: 196; Haseloff and Gerlach (1988) Nature 334: 585); Rossi et al. (1991) Pharmac. Ther. 50: 245-254) or a “hairpin” (see, e.g., U.S. Pat. No. 5,254,678 and Hampel et al., European Patent Publication No. 0 360 257, published Mar. 26, 1990; Hampel et al. (1990) Nucl. Acids Res. 18: 299-304), and have the ability to specifically target and cleave non-coding polynucleotides.

The sequence requirement for the hairpin ribozyme is any RNA sequence consisting of NNNBN*GUCNNNNNN (where N*G is the cleavage site, where B is any of G, C, or U, and where N is any of G, U, C, or A) (SEQ ID NO: 1). Suitable recognition or target sequences for hairpin ribozymes can be readily determined from the non-coding polynucleotide sequence of interest.

The sequence requirement at the cleavage site for the hammerhead ribozyme is any RNA sequence consisting of NUX (where N is any of G, U, C, or A and X represents C, U, or A). Accordingly, the same target within the hairpin leader sequence, GUC, is useful for the hammerhead ribozyme. The additional nucleotides of the hammerhead ribozyme or hairpin ribozyme are determined by the target flanking nucleotides and the hammerhead consensus sequence (see Ruffner et al. (1990) Biochemistry 29: 10695-10702).

Cech et al. (U.S. Pat. No. 4,987,071,) has disclosed the preparation and use of certain synthetic ribozymes which have endoribonuclease activity. These ribozymes are based on the properties of the Tetrahymena ribosomal RNA self-splicing reaction and require an 8-base pair target site. A temperature optimum of 50° C. is reported for the endoribonuclease activity. The fragments that arise from cleavage contain 5′ phosphate and 3′ hydroxyl groups and a free guanosine nucleotide added to the 5′ end of the cleaved RNA. Preferred ribozymes of the invention hybridize efficiently to target sequences at physiological temperatures, making them particularly well suited for use in vivo.

Ribozymes, as well as DNA encoding such ribozymes, and other suitable polynucleotide molecules can be chemically synthesized using methods well known in the art for the synthesis of polynucleotide molecules. After synthesis, the ribozyme can be modified by ligation to a DNA molecule having the ability to stabilize the ribozyme and make it resistant to RNase. Alternatively, as noted above, the ribozyme can be modified to the corresponding phosphothio analog for use, e.g., in liposome delivery systems. This modification also renders the ribozyme resistant to endonuclease activity. Promega, Madison, Wis., USA, provides a series of protocols suitable for the production of RNA molecules such as ribozymes.

Ribozymes also can be prepared from a DNA molecule or other polynucleotide molecule (which, upon transcription, yields an RNA molecule) operably linked to an RNA polymerase promoter, e.g., the promoter for T7 RNA polymerase or SP6 RNA polymerase. Accordingly, also provided by this invention are polynucleotide molecules, e.g., DNA or cDNA, coding for the ribozymes of this invention. When the vector also contains an RNA polymerase promoter operably linked to the polynucleotide molecule, the ribozyme can be produced in vitro upon incubation with the RNA polymerase and appropriate ribonucleotides. In a separate embodiment, the DNA may be inserted into an expression cassette (see, e.g., Cotten and Birnstiel (1989) EMBO J. 8(12):3861-3866; Hempel et al. (1989) Biochem. 28: 4929-4933, etc.).

When a vector containing an encoded ribozyme linked to a promoter for RNA transcription is introduced into a target cell, the RNA can be produced in the target cell when the target cell is grown under suitable conditions favoring transcription of the vector. The vector can be, but is not limited to, a plasmid, a virus, a retrotransposon or a cosmid. Examples of such vectors are disclosed in U.S. Pat. No. 5,166,320. Other representative vectors include, but are not limited to adenoviral vectors (e.g., WO 94/26914, WO 93/9191; Kolls et al. (1994) PNAS 91(1):215-219; Kass-Eisler et al., (1993) Proc. Natl. Acad. Sci., USA, 90(24): 11498-502, Guzman et al. (1993) Circulation 88(6): 2838-48, 1993; Guzman et al. (1993) Cir. Res. 73(6):1202-1207, 1993; Zabner et al. (1993) Cell 75(2): 207-216; Li et al. (1993) Hum Gene Ther. 4(4): 403-409; Caillaud et al. (1993) Eur. J. Neurosci. 5(10): 1287-1291), adeno-associated vector type 1 (“AAV-1”) or adeno-associated vector type 2 (“AAV-2”) (see WO 95/13365; Flotte et al. (1993) Proc. Natl. Acad. Sci., USA, 90(22):10613-10617), retroviral vectors (e.g., EP 0 415 731; WO 90/07936; WO 91/02805; WO 94/03622; WO 93/25698; WO 93/25234; U.S. Pat. No. 5,219,740; WO 93/11230; WO 93/10218) and herpes viral vectors (e.g., U.S. Pat. No. 5,288,641). Methods of utilizing such vectors in gene therapy are well known in the art, see, for example, Larrick and Burck (1991) Gene Therapy: Application of Molecular Biology, Elsevier Science Publishing Co., Inc., New York, N.Y., and Kreigler (1990) Gene Transfer and Expression: A Laboratory Manual, W.H. Freeman and Company, New York.

To produce ribozymes in vivo utilizing such vectors, the nucleotide sequence endoding the ribozyme is preferably operably linked to a strong promoter such as the lac, SV40 late, SV40 early, or lambda promoters.

Ribozymes, or polynucleotides encoding them (e.g., DNA vectors), can be formulated, and administered to cells, tissues, or organisms in accordance with standard practice. General considerations with respect to administration and dose are discussed below. Formulations containing at least one component that facilitates entry of a polynucleotide into a cell are as discussed above with respect to the administration of non-coding polynucleotides to cells to increase the level of non-coding polynucleotides. Those of skill in the art will readily appreciate that ribozymes, or polynucleotides encoding them, can be introduced into host cells as described above for non-coding polynucleotides.

### (2) Catalytic DNAs

In a manner analogous to ribozymes, DNA molecules are also capable of catalytic (e.g. nuclease) activity and can be employed in the method of the invention to reduce the level of non-coding polynucleotides. For example, highly catalytic species have been developed by directed evolution and selection. Beginning with a population of 1014 DNAs containing 50 random nucleotides, successive rounds of selective amplification enriched for individuals that best promote the Pb²⁺-dependent cleavage of a target ribonucleoside 3′-O—P bond embedded within an otherwise all-DNA sequence. By the fifth round, the population as a whole carried out this reaction at a rate of 0.2 min⁻¹. Based on the sequence of 20 individuals isolated from this population, a simplified version of the catalytic domain that operates in an intermolecular context with a turnover rate of 1 min⁻¹ (see, e.g., Breaker and Joyce (1994) Chem Biol 4: 223-229.

In later work, using a similar strategy, a DNA enzyme was made that could cleave almost any targeted RNA substrate under simulated physiological conditions. The enzyme is composed of a catalytic domain of 15 deoxynucleotides, flanked by two substrate-recognition domains of seven to eight deoxynucleotides each. The RNA substrate is bound through Watson-Crick base pairing and is cleaved at a particular phosphodiester located between an unpaired purine and a paired pyrimidine residue. Despite its small size, the DNA enzyme has a catalytic efficiency (kcat/Km) of approximately 10⁹ M⁻¹ min⁻¹ under multiple turnover conditions, exceeding that of any other known polynucleotide enzyme. By changing the sequence of the substrate-recognition domains, the DNA enzyme can be made to target different RNA substrates (Santoro and Joyce (1997) Proc. Natl. Acad. Sci., USA, 94(9): 4262-4266). Modifying the appropriate targeting sequences (e.g. as described by Santoro and Joyce, supra.) the DNA enzyme can easily be retargeted to a non-coding polynucleotide of interest and can be used in essentially the same manner as described above for ribozymes.

b. RNAi Methods

Another approach to reducing the level of non-coding polynucleotides entails RNA interference (RNAi). RNAi, also termed post-transcriptional gene silencing (PTGS), refers to a mechanism by which double-stranded (sense strand) RNA (dsRNA) specifically blocks expression of its homologous gene when injected, or otherwise introduced into cells. This approach is based on the observation that injection of antisense or sense RNA strands into C. elegans cells resulted in gene-specific inactivation (Guo and Kempheus (1995) Cell 81: 611-620). While gene inactivation by the antisense strand was expected, gene silencing by the sense strand was unexpected. Surprisingly, it was determined that the gene-specific inactivation was actually due to trace amounts of contaminating dsRNA (Fire et al. (1998) Nature 391: 806-811).

Since then, this mode of post-transcriptional gene silencing has been demonstrated in a wide variety of organisms: plants, flies, trypanosomes, planaria, hydra, zebrafish, and mice (Zamore et al. (2000) Cell 101: 25-33; Gura (2000) Nature 404: 804 808). RNAi activity has been associated with functions as disparate as transposon-silencing, anti-viral defense mechanisms, and gene regulation (Grant (1999) Cell 96: 303-306).

It has been shown that dsRNA is cleaved by a nuclease into 21-23-nucleotide fragments. These fragments, in turn, target the homologous region of their corresponding mRNA, hybridize, and result in a double-stranded substrate for a nuclease that degrades it into fragments of the same size (Hammond et al. (2000) Nature 404:293-298; Zamore et al. (2000) Cell 101:25-33). Although typically employed to target coding RNA (mRNA), this strategy is equally applicable to non-coding RNA.

dsRNA can be formulated and administered to cells, tissues, or organisms in accordance with standard practice. General considerations with respect to administration and dose are discussed below. Formulations containing at least one component that facilitates entry of a polynucleotide into a cell are as discussed above with respect to the administration of non-coding polynucleotides to cells to increase the level of non-coding polynucleotides. Those of skill in the art will readily appreciate that dsRNA can be introduced into host cells as described above for non-coding polynucleotides. Additionally, dsRNA can be synthesized using one or more vectors designed to transcribe the two complementary RNA strands that hybridize to form the dsRNA. These may be introduced into host cells using any of the techniques described herein or known in the art for this purpose.

c. “Knock-Out” Methods

In another approach, the level of a non-coding polynucleotide of interest can be reduced simply by “knocking out” the corresponding sequence in the CE. Typically, this is accomplished by disrupting the sequence, the promoter transcribing the sequence or sequences between the promoter and the sequence. Such disruption can be specifically directed to the selected sequence by homologous recombination where a “knockout construct” contains flanking sequences complementary to the domain to which the construct is targeted. Insertion of the knockout construct results in disruption of the selected sequence. By way of example, a nucleic acid construct can be prepared containing a DNA sequence encoding an antibiotic resistance gene which is inserted into the DNA sequence that is complementary to the DNA sequence to be disrupted. When this nucleic acid construct is then transfected into a cell, the construct will integrate into the genomic DNA. Thus, the cell and its progeny will no longer express the gene or will express it at a decreased level, as the DNA is now disrupted by the antibiotic resistance gene.

Knockout constructs can be produced by standard methods known to those of skill in the art. The knockout construct can be chemically synthesized or assembled, e.g., using recombinant DNA methods. The genomic DNA sequence to be used in producing the knockout construct is digested with a particular restriction enzyme selected to cut at a location(s) such that a new DNA sequence encoding, e.g., a marker gene can be inserted in the proper position within this DNA sequence. The proper position for marker gene insertion is that which will serve to reduce or prevent transcription of the targeted sequence; this position will depend on various factors such as the restriction sites in the sequence to be cut, and the precise location of insertion necessary to inhibit transcription of the sequence. Preferably, the enzyme selected for cutting the DNA will generate a longer arm and a shorter arm, where the shorter arm is at least about 300 base pairs (bp). In some cases, it will be desirable to actually remove a portion of the sequence to be suppressed so as to keep the length of the knockout construct comparable to the original genomic sequence when the marker gene is inserted in the knockout construct. In these cases, the genomic DNA is cut with appropriate restriction endonucleases such that a fragment of the proper size can be removed.

The marker gene can be any nucleic acid sequence that is detectable and/or assayable; however, typically it is an antibiotic resistance gene or other gene whose expression or presence in the genome can easily be detected. The marker gene is usually operably linked to its own promoter or to another strong promoter from any source that will be active, or can easily be activated, in the cell into which it is introduced; however, the marker gene need not be linked to its own promoter as it may be transcribed using the promoter of the sequence to be suppressed. In addition, the marker gene will normally have a polyA sequence attached to the 3′ end of the gene; this sequence serves to terminate transcription of the gene. Preferred marker genes include any antibiotic resistance gene such as, e.g., neo (the neomycin resistance gene) and beta-gal (beta-galactosidase).

After the genomic DNA sequence has been digested with the appropriate restriction enzymes, the marker gene sequence is ligated into the genomic DNA sequence using methods well known to the skilled artisan (see, e.g., Berger and Kimmel, Guide to Molecular Cloning Techniques, Methods in Enzymology volume 152 Academic Press, Inc., San Diego, Calif.; Sambrook et al. (1989) Molecular Cloning—A Laboratory Manual (2nd ed.) Vol. 1-3, Cold Spring Harbor Laboratory, Cold Spring Harbor Press, NY; and Current Protocols in Molecular Biology, F. M. Ausubel et al., eds., Current Protocols, a joint venture between Greene Publishing Associates, Inc. and John Wiley & Sons, Inc., (1994) Supplement).

The resulting knockout constructs can be delivered to cells in vivo using gene therapy delivery vehicles (e.g., retroviruses, liposomes, lipids, dendrimers, etc.). Methods of knocking out genes are well described in the literature and essentially routine to those of skill in the art (see, e.g., Thomas et al. (1986) Cell 44(3): 419-428; Thomas, et al. (1987) Cell 51(3): 503-512)1; Jasin and Berg (1988) Genes & Development 2: 1353-1363; Mansour, et al. (1988) Nature 336: 348-352; Brinster, et al. (1989) Proc Natl Acad Sci 86: 7087-7091; Capecchi (1989) Trends in Genetics 5(3): 70-76; Frohman and Martin (1989) Cell 56: 145-147; Hasty, et al. (1991) Mol Cell Bio 11(11): 5586-5591; Jeannotte, et al. (1991) Mol Cell Biol. 11(11): 557814 5585; and Mortensen, et al. (1992) Mol Cell Biol. 12(5): 2391-2395.

The use of homologous recombination to alter expression of endogenous genes is also described in detail in U.S. Pat. No. 5,272,071, WO 91/09955, WO 93/09222, WO 96/29411, WO 95/31560, and WO 91/12650.

Although embryonic stem (ES) cells can be employed to produce knockout animals, ES cells are not required. In various embodiments, knockout animals can be produced using methods of somatic cell nuclear transfer. In preferred embodiments using such an approach, a somatic cell is obtained from the species in which the sequence is to be knocked out. The cell is transfected with a construct that introduces a disruption in the sequence (e.g., via homologous recombination). Cells harboring a knocked out sequence are selected, e.g., by selecting for expression of a marker encoded by a marker gene used to disrupt the native sequence. The nucleus of cells harboring the knockout is then placed in an unfertilized enucleated egg (e.g., eggs from which the natural nuclei have been removed by microsurgery). Once the transfer is complete, the recipient eggs contain a complete set of genes, just as they would if they had been fertilized by sperm. The eggs are then cultured for a period before being implanted into a host mammal (of the same species that provided the egg) where they are carried to term, culminating in the birth of a transgenic animal containing a knocked-out sequence.

The production of viable cloned mammals following nuclear transfer of cultured somatic cells has been reported for a wide variety of species including, but not limited to frogs (McKinnell (1962) J. Hered. 53, 199-207), calves (Kato et al. (1998) Science 262: 2095-2098), sheep (Campbell et al. (1996) Nature 380: 64-66), mice (Wakayamaand Yanagimachi (1999) Nat. Genet. 22: 127-128), goats (Baguisi et al. (1999) Nat. Biotechnol. 17: 456-461), monkeys (Meng et al. (1997) Biol. Reprod. 57: 454-459), and pigs (Bishop et al. (2000) Nature Biotechnology 18: 1055-1059). Nuclear transfer methods have also been used to produce clones of transgenic animals. Thus, for example, the production of transgenic goats carrying the human antithrobin III gene by somatic cell nuclear transfer has been reported (Baguisi et al. (1999) Nature Biotechnology 17: 456-461).

Somatic cell nuclear transfer simplifies transgenic procedures by employing a differentiated cell source that can be clonally propagated. This eliminates the need to maintain the cells in an undifferentiated state, thus, genetic modifications, both random integration and gene targeting, are more easily accomplished. Also, by combining nuclear transfer with the ability to modify and select for these cells in vitro, this procedure is more efficient than previous transgenic embryo techniques.

Nuclear transfer techniques or nuclear transplantation techniques are known in the literature. See, in particular, Campbell et al. (1995) Theriogenology, 43:181; Collas et al. (1994) Mol. Report. Dev., 38:264-267; Keefer et al. (1994) Biol. Reprod., 50:935-939; Sims et al. (1993) Proc. Natl. Acad. Sci., USA, 90:6143-6147; WO 94/26884; WO 94/24274, WO 90/03432, U.S. Pat. Nos. 5,945,577, 4,944,384, 5,057,420 and the like.

### G. Modulation of the Level of the Specific Binding of the Non-Coding Polynucleotide to the Target Gene

a. Antisense Methods

The level of specific binding of the non-coding polynucleotide to the CE of the target gene can be reduced, for example, by the use of antisense molecules. An “antisense sequence or antisense polynucleotide” is a polynucleotide that is complementary to the non-coding polynucleotide sequence or a subsequence thereof. Binding of the antisense molecule to the non-coding polynucleotide can interfere with specific binding of the non-coding polynucleotide to the CE and/or, in some cases, to the epigenetic regulator.

Thus, in particular embodiments, the invention provides antisense molecules useful for inhibiting binding of the non-coding polynucleotide. Suitable antisense molecules include oligonucleotides and oligonucleotide analogs that are hybridizable with the non-coding polynucleotide of interest. Such oligonucleotides include, for example, polynucleotides formed from naturally-occurring bases and/or cyclofuranosyl groups joined by native phosphodiester bonds. The term “oligonucleotide” encompasses moieties that function similarly to oligonucleotides, but that have non-naturally occurring portions. Thus, oligonucleotides may have altered sugar moieties or inter-sugar linkages. Exemplary among these are the phosphorothioate and other sulfur containing species that are known for use in the art. In accordance with some preferred embodiments, at least one of the phosphodiester bonds of the oligonucleotide has been substituted with a structure which functions to enhance the ability of the compositions to penetrate into the region of cells where the non-coding polynucleotide whose activity is to be modulated is located. It is preferred that such substitutions comprise phosphorothioate bonds, methyl phosphonate bonds, or short-chain alkyl or cycloalkyl structures. In accordance with other preferred embodiments, the phosphodiester bonds are substituted with structures that are, at once, substantially non-ionic and non-chiral, or with structures which are chiral and enantiomerically specific. Persons of ordinary skill in the art will be able to select other linkages for use in the practice of the invention.

In an exemplary embodiment, the internucleotide phosphodiester linkage is replaced with a peptide linkage. Such peptide polynucleotides tend to show improved stability, penetrate the cell more easily, and show enhanced affinity for their target. Methods of making peptide polynucleotides are known to those of skill in the art (see, e.g., U.S. Pat. Nos. 6,015,887, 6,015,710, 5,986,053, 5,977,296, 5,902,786, 5,864,010, 5,786,461, 5,773,571, 5,766,855, 5,736,336, 5,719,262, and 5,714,331).

Oligonucleotides useful in the antisense methods of the invention may also include one or more modified base forms. Thus, purines and pyrimidines other than those normally found in nature may be employed. Similarly, the furanosyl portions of the nucleotide subunits may also be modified, as long as the essential tenets of this invention are adhered to. Examples of such modifications are 2′-O-alkyl- and 2′-halogen-substituted nucleotides. Some specific examples of modifications at the 2′ position of sugar moieties which are useful in the present invention are: OH, SH, SCH₃, F, OCH₃, OCN, O(CH₂)[n]NH₂ or O(CH₂)[n]CH₃, where n is from 1 to about 10, and other substituents having similar properties.

All such analogs can be used in the antisense methods of the invention so long as the analogs function effectively to hybridize with the non-coding polynucleotide of interest and inhibit its function.

Antisense oligonucleotides in accordance with this invention preferably comprise from about 3 to about 50 subunits (i.e., bases in unmodified polynucleotides). It is more preferred that such oligonucleotides and analogs comprise from about 8 to about 25 subunits and still more preferred to have from about 12 to about 25 subunits. The oligonucleotides used in accordance with this invention can be conveniently and routinely made through the well-known technique of solid phase synthesis. Equipment for such synthesis is sold by several vendors (e.g., Applied Biosystems).

Antisense oligonucleotides of the invention can be synthesized, formulated, and administered to cells, tissues, or organisms in accordance with standard practice. General considerations with respect to administration and dose are discussed below. Formulations containing at least one component that facilitates entry of a polynucleotide into a cell are as discussed above with respect to the administration of non-coding polynucleotides to cells to increase the level of non-coding polynucleotides. Those of skill in the art will readily appreciate that antisense molecules can be introduced into host cells as described above for non-coding polynucleotides.

H. Modulation of the Level of the Specific Binding of the Epigenetic Regulator to the Non-Coding Polynucleotide

a. Antisense Methods

Antisense molecules according to the invention can, as noted above, be employed reduce the level of specific binding of the epigenetic regulator to the non-coding polynucleotide. In certain embodiments, the antisense molecule inhibits this binding by disrupting a secondary structure in the non-coding polynucleotide that is required for, or contributes to, the binding of the epigenetic regulator. Antisense molecules that inhibit this binding with the desired specificity can be designed and easily tested in a standard binding assay (see section V, below).

b. Intrabodies

In another embodiment, the binding of the epigenetic regulator to the non-coding polynucleotide can be inhibited by introducing a nucleic acid construct that expresses an intrabody into the target cells. An intrabody is an intracellular antibody, in this case, capable of recognizing and binding to an epigenetic regulator of interest. The intrabody is expressed by an “antibody cassette” containing: (1) a sufficient number of nucleotides encoding the portion of an antibody capable of binding to the target (the epigenetic regulator of interest) operably linked to (2) a promoter that will permit expression of the antibody in the cell(s) of interest. The construct encoding the intrabody is delivered to the cell where the antibody is expressed intracellularly and binds to the target epigenetic regulator, thereby disrupting the target from its normal action.

In a preferred embodiment, the “intrabody gene” of the antibody cassette includes a cDNA encoding heavy chain variable (V_{H}) and light chain variable (V_{L}) domains of an antibody which can be connected at the DNA level by an appropriate oligonucleotide linker, which on translation, forms a single peptide (referred to as a single chain variable fragment, “sFv”) capable of binding to a target such as an epigenetic regulator. The intrabody gene preferably does not encode an operable secretory sequence, and thus the expressed antibody remains within the cell.

Anti-epigenetic regulator antibodies suitable for use/expression as intrabodies in the methods of this invention can be readily produced by a variety of methods. Such methods include, but are not limited to, traditional methods of raising polyclonal antibodies, which can be modified to form single chain antibodies, or screening of, e.g., phage display libraries to select for antibodies showing high specificity and/or avidity for the target epigenetic regulator.

The antibody cassette is delivered to the cell by any means suitable for introducing polynucleotides into cells. A preferred delivery system is described in U.S. Pat. No. 6,004,940. Methods of making and using intrabodies are described in detail in U.S. Pat. Nos. 6,072,036, 6,004,940, and 5,965,371.

c. Mutant Epigentic Regulators

Another approach for reducing the level of specific binding of the epigenetic regulator to the non-coding polynucleotide entails the use of mutant epigenetic regulators. In particular, a mutant epigenetic regulator can be introduced into cells to competitively inhibit the binding of the native epigenetic regulator to the non-coding polynucleotide. In this embodiment, the mutant epigenetic regulator retains the ability to bind to the non-coding polynucleotide, but lacks a function necessary for modulating transcription of the target gene. Exemplary mutants useful in this embodiment include, in the case of regulators that act by modifying histone proteins, mutant regulators that lack the enzymatic activity to produce these modifications. Thus, for example, if the epigenetic regulator is a histone methyltransferase, a mutant useful in the embodiment could bear a mutation that reduces or eliminates the methyltransferase activity. Such mutants can be full-length proteins, but need not be. Fragments, such as, for example, the SET-domain, can be employed so long as they retain the capacity to compete with the native epigenetic regulator for binding to the desired non-coding polynucleotide. Examples of Ash1 mutants that lack histone methyltransferase activity include Ash110, Ash121, and Ash122.

Mutant epigenetic regulators can be administered to cells by any means capable of delivering the mutant to the desired site of action, namely the corresponding non-coding polynucleotide. This can be accomplished, for example, using a construct capable of expressing the mutant epigenetic regulator intracellularly, as described above for intrabodies.

I. Modulators

Any modulator that alters the level of: (1) the non-coding polynucleotide; (2) the specific binding of the non-coding polynucleotide to the target gene; and/or (3) the specific binding of the epigenetic regulator to the non-coding polynucleotide can be employed in this method, provided the modulator can be introduced into the target cells without undue toxicity. In addition to polynucleotides and polypeptides, small-molecule modulators can be identified in one or more of the screening methods described below and used to regulate transcription, as described above.

J. Applications

The above-described method can be employed to regulate transcription of a suitable target gene in any setting in which such regulation is desired. Thus, the method is useful in research or diagnostic applications in which the consequences of such regulation are of interest, as well as in therapeutic applications.

Modulators according to the invention, can formulated for use in assays and/or administration to cells, tissues, or organisms. The compositions optionally contain other components, including, for example, a storage solution, such as a suitable buffer, e.g., a physiological buffer. In a preferred embodiment, the composition is a pharmaceutical composition and the other component is a pharmaceutically acceptable carrier, such as are described in Remington's Pharmaceutical Sciences (1980) 16th editions, Osol, ed., 1980. The composition optionally includes at least one component that facilitates cell entry by the modulator. Components that facilitate entry of small molecules, peptides, polypeptides, oligonucleotides, and polynucleotides are known in the art and can be used in the invention (see above for descriptions of components that facilitate cell entry by polynucleotides).

For in vitro applications, cells are contacted with a modulator of the invention simply by adding the modulator directly to the medium of cultured cells or directly to tissues.

Methods for in vivo administration do not differ from known methods for administering small-molecule drugs or therapeutic peptides, polypeptides, oligonucleotides, or polynucleotides. Suitable routes of administration include, for example, topical, intravenous, intraperitoneal, intracerebral, intramuscular, intraocular, intraarterial, or intralesional routes. Pharmaceutical compositions of the invention can be administered continuously by infusion, by bolus injection, or, where the compositions are sustained-release preparations, by methods appropriate for the particular preparation.

The dose of modulator is sufficient to regulate transcription without undue toxicity. For in vivo applications, the dose of modulator depends, for example, upon the therapeutic objectives, the route of administration, and the condition of the subject. It is routine for the clinician to titer the dosage and modify the route of administration as required to obtain the optimal therapeutic effect. Generally, the clinician begins with a low dose and increases the dosage until the desired therapeutic effect is achieved. Starting doses for a given modulator can be extrapolated from in vitro data.

The specific application will vary depending upon the target gene. If, for example, the target gene is one that has a role in cell proliferation and/or cell differentiation, a modulator can be employed according to the method of the invention to modulate cell proliferation and/or cell differentiation. In particular embodiments, for example, the modulator can be administered to a cancer cell (e.g., to inhibit proliferation), a dormant cell (e.g., to stimulate proliferation), or a stem cell (e.g., to modulate differentiation).

In an exemplary in vivo embodiment, the method is carried out by administering a composition comprising the modulator to a subject having a condition treatable by modulation of cell proliferation and/or cell differentiation, such as a cancer patient. In this embodiment, the modulator generally either represses the transcription of a target gene that stimulates cell proliferation or activates the transcription of a target gene that suppresses cell proliferation. Other exemplary in vivo embodiments include those in which the method is carried out to promote wound healing, e.g., to treat non-healing wounds in subject with diabetes or to treat burn victims. The method can also be used to treat neurodegenerative disease (such as, e.g., Alzheimers and Parkinsons), paralysis, tissue failure (e.g., affecting the eye or skin), organ failure (e.g., affecting the kidney, stomach, lung, heart, pancreas, or liver), osteoporosis, and muscular dystrophy.

In certain embodiments, the method can be carried out “ex vivo” to treat such conditions. In particular, cells, a tissue, or an organ is removed from a patient, treated as described above for in vitro applications, and then reimplanted into the patient using standard techniques.

An important in vitro application of the method of the invention is its use to induce cell differentiation. In particular, by activating and/or repressing the transcription of genes that play a role in the differentiation of unspecialized to specialized cells, cells having desired phenotypes can be produced in vitro. Thus, for example, one or more non-coding polynucleotides can be introduced into a stem cell to induce its differentiation to a skin cell. This embodiment allows the preparation of cells and tissues for study and/or grafting, implantation, or transplantation.

II. Characterization of Transcriptional Activity of Genes that are Targets for Epigenetic Regulators

A. In General

The invention also provides a method of characterizing the transcriptional activity of a gene that is a target for an epigenetic regulator. The method is applicable to any target gene that has a cis-regulatory region including a chromosomal element (CE) for the epigenetic regulator, wherein the CE includes a sequence that is a template for a non-coding polynucleotide. The non-coding polynucleotide recruits the epigenetic regulator to the CE, which either activates or represses transcription of the target gene.

The method is carried out using a biological sample that includes the gene and the epigenetic regulator and entails determining whether the non-coding polynucleotide is present in the biological sample. In preferred embodiments, the method additionally includes determining whether the non-coding polynucleotide is physically associated with the CE and the epigenetic regulator (e.g., using in vivo cross-linked chromatin immunoprecipitation, as described in greater detail below). In one embodiment, the amount of non-coding polynucleotide present in a test sample is compared with the amount of non-coding in a control or reference sample. This embodiment provides an indication of the transcriptional activity of the target gene in the test sample relative to the control/reference sample. In a variation of this embodiment, the amount of non-coding polynucleotide physically associated with the CE and the epigenetic regulator in the test sample is compared with the amount of non-coding polynucleotide physically associated with the CE and the epigenetic regulator in the control sample.

The ability to conveniently characterize the transcriptional activity of a target gene has a wide variety of applications. In particular, if the transcriptional activity of the target gene is correlated with an abnormal condition, the method can be carried out to identify, or assist in identifying, the presence of the abnormal condition. Thus, for example, if the epigenetic regulator is an activator for a target gene that is normally silent in the tissue or cell being assayed, the presence of the non-coding polynucleotide that recruits the epigenetic activator to the target gene indicates abnormally high transcription of the target gene, signaling the presence of a corresponding abnormal condition. Similarly, if the epigenetic regulator is a repressor for a target gene that normally active in the tissue or cell being assayed, the present of the non-coding polynucleotide that recruits the epigenetic repressor to the target gene indicates abnormally low transcription of the target gene, also signaling the presence of an abnormal condition. In such embodiments, the difference between the amount of non-coding polynucleotide present (or more preferably, physically associated with the CE and the epigenetic regulator) in a test sample, compared with the amount of non-coding polynucleotide present (or physically associated with the CE and the epigenetic regulator) in a control sample, provides a metric useful in the diagnosis and/or prognosis of the abnormal condition. In exemplary embodiments, the target gene is one that plays a role in cell proliferation, and the abnormal condition includes abnormal cell proliferation. This embodiment is useful, for example, in the early diagnosis of cancer, by detecting abnormal transcriptional activity leading to cell proliferation, rather than abnormal proliferation per se.

In another embodiment, the transcriptional activity of the target gene is correlated with a particular cell type, and the non-coding polynucleotide is detected as an indicator of cell type-specific transcriptional activity, which can be used, alone or together with other markers, to identify a particular cell type. Similarly, if the transcriptional activity of the target gene is correlated with a particular stage of cell differentiation, the non-coding polynucleotide can be detected as an indicator of that stage.

Target genes useful in the method include those described above in section I.B. In particular embodiments, the target gene is a homeotic gene.

The epigenetic regulator is as described above in section I.C. In specific embodiments, the epigenetic regulator is a histone methyltransferase and/or includes a SET-module. The epigenetic regulator can be one that activates transcription, or one that represses transcription, of the target gene; examples of each are discussed above in section I.C.

The non-coding polynucleotide includes those described above in section I.D. and is typically, though not necessarily, RNA.

The biological sample can include any type of tissue, cell, or cell fraction containing the necessary component(s) and can be obtained from any multicellular organism, as described above in section I.E. Tissues, cells, or cell fractions employed in the method can be obtained from a living organism or cultured cells or tissue. In particular embodiments, the biological sample includes mammalian cells, and more particularly, human cells.

Non-coding polynucleotides can be detected in a suitable sample directly or after purification of sample polynucleotides, depending on the assay method employed. Polynucleotides can be purified from a sample according to any of a number of methods well known to those of skill in the art. General methods for isolation and purification of polynucleotides are described in detail in by Tijssen ed., (1993) Chapter 3 of Laboratory Techniques in Biochemistry and Molecular Biology: Hybridization With Nucleic Acid Probes, Part I Theory and Nucleic Acid Preparation, Elsevier, N.Y. and Tijssen ed. If the non-coding polynucleotide is RNA, its presence can be detected by detecting the RNA or by detecting the presence of a polynucleotide derived from the mRNA (e.g., amplified, reverse-transcribed cDNA, etc.).

B. Amplification-Based Assays

In one embodiment, amplification-based assays can be used to detect, and optionally quantify, a non-coding polynucleotide. In exemplary amplification-based assays, a non-coding polynucleotide in the sample acts as a template in an amplification reaction carried out with a nucleic acid primer that contains a detectable label or component of a labeling system. Suitable amplification methods include, but are not limited to, polymerase chain reaction (PCR); reverse-transcription PCR(RT-PCR); ligase chain reaction (LCR) (See Wu and Wallace (1989) Genomics 4: 560, Landegren et al. (1988) Science 241: 1077, and Barringer et al. (1990) Gene 89: 117; transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874); dot PCR, and linker adapter PCR, etc.

To determine the level of non-coding polynucleotide, any of a number of well known “quantitative” amplification methods can be employed. Quantitative PCR generally involves simultaneously co-amplifying a known quantity of a control sequence using the same primers. This provides an internal standard that may be used to calibrate the PCR reaction. Detailed protocols for quantitative PCR are provided in PCR Protocols, A Guide to Methods and Applications, Innis et al., Academic Press, Inc. N.Y., (1990).

C. Hybridization-Based Assays

In another embodiment, the non-coding polynucleotide can be detected by nucleic acid hybridization. Nucleic acid hybridization simply involves contacting a nucleic acid probe with sample polynucleotides under conditions where the probe and its complementary target nucleotide sequence can form stable hybrid duplexes through complementary base pairing. The nucleic acids that do not form hybrid duplexes are then washed away leaving the hybridized nucleic acids to be detected, typically through detection of an attached detectable label or component of a labeling system. Methods of detecting and/or quantifying polynucleotides using nucleic acid hybridization techniques are known to those of skill in the art (see Sambrook et al. supra). Hybridization techniques are generally described in Hames and Higgins (1985) Nucleic Acid Hybridization, A Practical Approach, IRL Press; Gall and Pardue (1969) Proc. Natl. Acad. Sci. USA 63: 378-383; and John et al. (1969) Nature 223: 582-587.

In general, polynucleotides are denatured by increasing the temperature or decreasing the salt concentration of the buffer containing the polynucleotides, or in the addition of chemical agents, or the raising of the pH. Under low stringency conditions (e.g., low temperature and/or high salt and/or high target concentration) hybrid duplexes (e.g., DNA:DNA, RNA:RNA, or RNA:DNA) will form even where the annealed sequences are not perfectly complementary. Thus specificity of hybridization is reduced at lower stringency. Conversely, at higher stringency (e.g., higher temperature or lower salt) successful hybridization requires fewer mismatches.

One of skill in the art will appreciate that hybridization conditions may be selected to provide any degree of stringency. In general, there is a tradeoff between hybridization specificity (stringency) and signal intensity. In a preferred embodiment, the wash is performed at the highest stringency that produces consistent results and that provides a signal intensity greater than approximately 10% of the background intensity. Hybridization can performed at low stringency to ensure hybridization and then subsequent washes are performed to eliminate mismatched hybrid duplexes. Successive washes may be performed at increasingly higher stringency (e.g., down to as low as 0.25×SSPE at 37° C. to 70° C.) until a desired level of hybridization specificity is obtained. Stringency can also be increased by addition of agents such as formamide. Hybridization specificity may be evaluated by comparison of hybridization to the test probes with hybridization to the various controls that can be included in the reaction mixture.

Methods of optimizing hybridization conditions are well known to those of skill in the art (see, e.g., Tijssen (1993) Laboratory Techniques in Biochemistry and Molecular Biology, Vol. 24: Hybridization With Polynucleotide Probes, Elsevier, N.Y.). In a preferred embodiment, background signal is reduced by the use of a blocking reagent (e.g., tRNA, sperm DNA, cot-1 DNA, etc.) during the hybridization to reduce non-specific binding. The use of blocking agents in hybridization is well known to those of skill in the art (see, e.g., Chapter 8 in P. Tijssen, supra.)

The nucleic acid probes used herein for detection of a non-coding polynucleotide can be full-length or less than the full-length of these polynucleotides. Shorter probes are generally empirically tested for specificity. Preferably, nucleic acid probes are at least about 15, and more preferably about 20 bases or longer, in length. (See Sambrook et al. for methods of selecting nucleic acid probe sequences for use in nucleic acid hybridization.) Visualization of the hybridized probes allows the qualitative determination of the presence or absence of non-coding polynucleotide, and standard methods (such as, e.g., densitometry where the nucleic acid probe is radioactively labeled) can be used to quantify the level of non-coding polynucleotide).

A variety of nucleic acid hybridization formats are known to those skilled in the art. Standard formats include sandwich assays and competition or displacement assays. Sandwich assays are commercially useful hybridization assays for detecting or isolating polynucleotides. Such assays utilize a “capture” nucleic acid covalently immobilized to a solid support and a labeled “signal” nucleic acid in solution. The sample provides the target polynucleotide. The capture nucleic acid and signal nucleic acid each hybridize with the target polynucleotide to form a “sandwich” hybridization complex.

In one embodiment, the methods of the invention can be utilized in array-based hybridization formats. In an array format, a large number of different hybridization reactions can be run essentially “in parallel.” This provides rapid, essentially simultaneous, evaluation of a number of hybridizations in a single experiment. Methods of performing hybridization reactions in array-based formats are well known to those of skill in the art (See, e.g., Pastinen (1997) Genome Res. 7: 606-614; Jackson (1996) Nature Biotechnology 14:1685; Chee (1995) Science 274: 610; WO 96/17958, Pinkel et al. (1998) Nature Genetics 20: 207-211).

Arrays, particularly nucleic acid arrays, can be produced according to a wide variety of methods well known to those of skill in the art. For example, in a simple embodiment, “low-density” arrays can simply be produced by spotting (e.g., by hand using a pipette) different nucleic acids at different locations on a solid support (e.g., a glass surface, a membrane, etc.). This simple spotting approach has been automated to produce high-density spotted microarrays. For example, U.S. Pat. No. 5,807,522 describes the use of an automated system that taps a microcapillary against a surface to deposit a small volume of a biological sample. The process is repeated to generate high-density arrays. Arrays can also be produced using oligonucleotide synthesis technology. Thus, for example, U.S. Pat. No. 5,143,854 and PCT Patent Publication Nos. WO 90/15070 and 92/10092 teach the use of light-directed combinatorial synthesis of high-density oligonucleotide microarrays. Synthesis of high-density arrays is also described in U.S. Pat. Nos. 5,744,305; 5,800,992; and 5,445,934.

In a preferred embodiment, the arrays used in this invention contain “probe” nucleic acids. These probes are then hybridized respectively with their “target” nucleotide sequence(s) present in polynucleotides derived from a biological sample. Alternatively, the format can be reversed, such that polynucleotides from different samples are arrayed and this array is then probed with one or more probes, which can be differentially labeled.

Many methods for immobilizing nucleic acids on a variety of solid surfaces are known in the art. A wide variety of organic and inorganic polymers, as well as other materials, both natural and synthetic, can be employed as the material for the solid surface. Illustrative solid surfaces include, e.g., nitrocellulose, nylon, glass, quartz, diazotized membranes (paper or nylon), silicones, polyformaldehyde, cellulose, and cellulose acetate. In addition, plastics such as polyethylene, polypropylene, polystyrene, and the like can be used. Other materials that can be employed include paper, ceramics, metals, metalloids, semiconductive materials, and the like. In addition, substances that form gels can be used. Such materials include, e.g., proteins (e.g., gelatins), lipopolysaccharides, silicates, agarose and polyacrylamides. Where the solid surface is porous, various pore sizes may be employed depending upon the nature of the system.

In preparing the surface, a plurality of different materials may be employed, particularly as laminates, to obtain various properties. For example, proteins (e.g., bovine serum albumin) or mixtures of macromolecules (e.g., Denhardt's solution) can be employed to avoid non-specific binding, simplify covalent conjugation, and/or enhance signal detection. If covalent bonding between a compound and the surface is desired, the surface will usually be polyfunctional or be capable of being polyfunctionalized. Functional groups that may be present on the surface and used for linking can include carboxylic acids, aldehydes, amino groups, cyano groups, ethylenic groups, hydroxyl groups, mercapto groups and the like. The manner of linking a wide variety of compounds to various surfaces is well known and is amply illustrated in the literature.

Arrays can be made up of target elements of various sizes, ranging from about 1 mm diameter down to about 1 μm. Relatively simple approaches capable of quantitative fluorescent imaging of 1 cm² areas have been described that permit acquisition of data from a large number of target elements in a single image (see, e.g., Wittrup (1994) Cytometry 16:206-213, Pinkel et al. (1998) Nature Genetics 20: 207-211).

Hybridization assays according to the invention can also be carried out using a MicroElectroMechanical System (MEMS), such as the Protiveris' multicantilever array.

D. Polynucleotide Detection

The non-coding polynucleotide can be detected in the above-described polynucleotide-based assays by means of a detectable label. Detectable labels suitable for use in the present invention include any moiety or composition detectable by spectroscopic, photochemical, biochemical, immunochemical, electrical, optical or chemical means. Examples include biotin for staining with a labeled streptavidin conjugate, magnetic beads (e.g., Dynabeads™), fluorescent dyes (e.g., fluorescein, texas red, rhodamine, coumarin, oxazine, green fluorescent protein, and the like, see, e.g., Molecular Probes, Eugene, Oreg., USA), radiolabels (e.g., ³H, ¹²⁵I, ³⁵S, ¹⁴C, or ³²P), enzymes (e.g., horseradish peroxidase, alkaline phosphatase), and colorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

The label may be added to a probe or primer or sample polynucleotides prior to, or after, the hybridization or amplification. So called “direct labels” are detectable labels that are directly attached to or incorporated into the labeled polynucleotide prior to conducting the assay. In contrast, so called “indirect labels” are joined to the hybrid duplex after hybridization. In indirect labeling, one of the polynucleotides in the hybrid duplex carries a component to which the detectable label binds. Thus, for example, a probe or primer can be biotinylated before hybridization. After hybridization, an avidin-conjugated fluorophore can bind the biotin-bearing hybrid duplexes, providing a label that is easily detected. For a detailed review of methods of the labeling and detection of polynucleotides, see Laboratory Techniques in Biochemistry and Molecular Biology, Vol. 24: Hybridization With Nucleic Acid Probes, P. Tijssen, ed. Elsevier, N.Y., (1993)).

The sensitivity of the hybridization assays can be enhanced through use of a polynucleotide amplification system that multiplies the target polynucleotide being detected. Examples of such systems include the polymerase chain reaction (PCR) system and the ligase chain reaction (LCR) system. Other methods described in the art are the nucleic acid sequence based amplification (NASBAO, Cangene, Mississauga, Ontario) and Q Beta Replicase systems.

In a preferred embodiment, suitable for use in amplification-based assays of the invention, a primer contains two fluorescent dyes, a “reporter dye” and a “quencher dye.” When intact, the primer produces very low levels of fluorescence because of the quencher dye effect. When the primer is cleaved or degraded (e.g., by exonuclease activity of a polymerase, see below), the reporter dye fluoresces and is detected by a suitable fluorescent detection system. Amplification by a number of techniques (PCR, RT-PCR, RCA, or other amplification method) is performed using a suitable DNA polymerase with both polymerase and exonuclease activity (e.g., Taq DNA polymerase). This polymerase synthesizes new DNA strands and, in the process, degrades the labeled primer, resulting in an increase in fluorescence. Commercially available fluorescent detection systems of this type include the ABI Prism® Systems 7000, 7700, or 7900 (TaqMan®) from Applied Biosystems or the LightCycler® System from Roche.

E. Chromatin Immunoprecipitation

Preferred embodiments of the invention include a determination as to whether the non-coding polynucleotide is physically associated with the CE and epigenetic regulator of interest. This determination is most conveniently carried out using chromatin immunoprecipitation, including in vivo cross-linked chromatin immunoprecipitation (XchIP) and native chromatin immunoprecipitation. These techniques are illustrated in the Examples herein. Briefly, in XchIP, cells are incubated with cross-linker that cross-links the DNA and associated proteins present in chromatin. Any suitable cross-linker, such as formamide or formaldehyde can be employed. Other crosslinkers suitable for carrying out in vivo crosslinking are described, e.g., in U.S. Pat. No. 5,770,736 (filed Jun. 23, 1998) and U.S. Pat. No. 6,008,211 (filed Dec. 28, 1999). In the Examples, cross-linking was achieved by treating cells with 1.8% formaldehyde for 15 min. Chromatin was then isolated from the cells and sheared to a desired average fragment length, which facilitates handling of the chromatin and provides the desired degree of resolution (i.e., allowing one to conclude that a protein of interest is bound to a CE of interest, rather than a neighboring CE). In the Examples, herein, the chromatin was sheared to an average length of about 400 basepairs. Native chromatin immunoprecipitation is carried out in essentially the same manner, except that no crosslinker is used.

The sheared chromatin is then subjected to immunoprecipitation with an antibody of interest. The antibody is contacted with the chromatin under conditions suitable for antibody binding. In the present method, an antibody specific for the epigenetic regulator is employed. Immunoprecipitation results in the recovery of the antibody complexed with the epigenetic regulator and any associated non-coding polynucleotide and CE. Immunoprecipitation can be carried out using any conventional techniques, such as affinity chromatography over a column that binds the antibody, such as e.g. a Protein A column.

To examine CEs (DNA) that immunoprecipitate with the epigenetic regulator, antibody/chromatin complexes are incubated with an RNase and a proteinase (e.g., Proteinase K) to remove RNA and proteins, respectively, followed by a suitable treatment to reverse the cross-links. Where formaldehyde is used as the cross-linker, incubation at 65° C. for about 6 hours is sufficient to reverse the cross-links. To examine non-coding polynucleotides (typically RNA) that immunoprecipitate with the epigenetic regulator, antibody/chromatin complexes are incubated with DNase and Proteinase-K to remove DNA and proteins, followed by reversal of cross-links. Precipitated DNA and RNA can then purified and used as templates for amplification. For example, PCR and RT-PCR can be used detect the presence of precipitated DNA and RNAs, respectively, in generated nucleic acid pools.

III. Identification of Chromosomal Elements that are Targets for Epizenetic Regulators

Another aspect of the invention is a method of screening for a chromosomal element (CE) for an epigenetic regulator of a target gene, wherein the CE includes a sequence that is a template for a non-coding polynucleotide. This method can be carried out in any of a number of ways.

In one embodiment, screening for CEs can be carried out by determining whether a sequence of a putative CE is transcribed in a cell. This embodiment generally entails assaying cellular RNA to determine whether the sequence is present. Generally, in vitro assays are most convenient, including amplification- or hybridization-based methods, as described above. High-throughput methods, e.g., employing nucleic acid arrays are preferred. In such methods, putative templates from putative CEs can be arrayed on a substrate, followed by hybridization with cellular RNA to identify any transcribed sequences that hybridize to the putative template sequence(s).

In another embodiment, the CE screening method entails determining whether the epigenetic regulator is physically associated with a non-coding polynucleotide corresponding to a putative CE and/or physically associated with the putative CE. In certain embodiments, this method can be carried out using any suitable in vitro binding assay. Thus, screening assays can be carried out, for example, using purified or partially purified components, in cell lysates, in cultured cells, or in other biological samples. Means of assaying for specific binding of two or more binding partners are well known to those of skill in the art. In preferred binding assays, one binding partner is immobilized and exposed to the second binding partner (which can be labeled). The immobilized binding partner is then washed to remove any unbound material and the labeled binding partner is then detected. To screen large numbers of putative non-coding polynucleotides or putative CEs, high-throughput assays are generally preferred. In exemplary embodiments, putative non-coding polynucleotides can be arrayed on a substrate, which can then be contacted with the epigenetic regulator under conditions suitable for specific binding to one or more non-coding polynucleotides. Alternatively, putative CEs can be arrayed and then contacted with the epigenetic regulator under conditions that permit specific binding between the epigenetic regulator and any putative CEs. Generally, the epigenetic regulator is contacted with the arrayed CEs in the presence of one or more non-coding polynucleotides corresponding to putative template sequences within the arrayed CEs. For example, the epigenetic regulator can be contacted with the arrayed CEs in the presence of cellular RNA from a cell in which a target gene corresponding to one or more of the putative CEs is transcribed. This RNA is expected to contain the non-coding polynucleotide that mediates binding of the epigenetic regulator to the CE of the target gene. Thus, binding of the epigenetic regulator to one or more putative CEs of the array identifies them as candidates for controlling transcription of the target gene.

In another embodiment, the CE screening method entails determining whether a physical association between the epigenetic regulator and a non-coding polynucleotide corresponding to a putative CE and/or between the epigenetic regulator and the putative CE exists in a cell. This embodiment can be performed, for example, by in vivo cross-linked or native chromatin immunoprecipitation, as described above. In particular, an antibody specific for the epigenetic regulator believed to act at a putative CE can be used to immunoprecipitate any associated chromatin, followed by: (1) purification of RNA and amplification to determine the presence of a non-coding polynucleotide corresponding to the putative CE, (2) purification of DNA and amplification to determine the presence of a putative CE, or (3) both.

Alternatively, screening for CEs can be carried out by determining whether a non-coding polynucleotide corresponding to a putative CE mediates transcriptional regulation by the epigenetic regulator. In this embodiment, numerous non-coding polynucleotides, corresponding to different CEs, can be screening by introducing (e.g., by transfection or expression) non-coding polynucleotides into cells including the epigenetic regulator and the putative CE. The latter can be endogenous in the cell or introduced into the cell using standard recombinant techniques. Transcriptional regulation by the epigenetic regulator can be measured: (1) directly by assaying transcription of the target gene, using, e.g., an amplification- or hybridization-based assay, or (2) indirectly by assaying a biological response that is correlated with transcription of the target gene. Transcriptional regulation by the epigenetic regulator can also be assessed by linking the putative CE to a suitable reporter (i.e., easily assayed heterologous) gene, such as the firefly luciferase gene and transfecting this construct into the cell. If desired, expression of the epigenetic regulator can be placed under the control of an inducible promoter to allow assessment of transcriptional activity mediated by the non-coding polynucleotides in the presence and absence of the epigenetic regulator.

Non-coding polynucleotides corresponding to a putative CE can be selected for testing in the method based on sequence or structural comparison with known CE sequences. Alternatively, non-coding polynucleotides having sequences derived from chromosomal regions implicated in epigenetic regulation can be tested. Finally, entire libraries of sequences can be screened, to identify those that physically associate with an epigenetic regulator and/or a putative CE or that mediate transcriptional regulation by the epigenetic regulator.

The method is applicable to target genes, epigenetic regulators, non-coding polynucleotides, and cell types as described above for the other methods of the invention. Screening for CEs can be carried out in vivo or in vitro; however, in vitro screening assays, using cells in culture or cell factions, are generally most convenient.

### IV. Epigenetic Regulator-Non-coding Polynucleotide Complex

The invention also provides an isolated complex including an epigenetic regulator for a target gene, wherein the epigenetic regulator is specifically bound to a non-coding polynucleotide. The target gene is one that has a cis-regulatory region including a chromosomal element (CE) for the epigenetic regulator, and the CE includes a sequence that is a template for a non-coding polynucleotide. The non-coding polynucleotide is generally RNA.

The isolated complex can be obtained from any of the cell types discussed above by any suitable technique, but is most conveniently obtained from cells by chromatin immunoprecipitation. The complex can also include a CE corresponding to the non-coding polynucleotide, such as is produced upon chromatin immunoprecipitation. Alternatively, the CE can be removed from the immunoprecipitated complex, for example, by digestion with DNAse.

In another embodiment, the complex can be obtained by contacting the purified preparations of the epigenetic regulator and non-coding polynucleotide under conditions that allow complex formation. In this embodiment, the epigenetic regulator can be produced using standard recombinant techniques, purified from a natural source, or synthesized.

For recombinant production, host cells transformed with expression vectors can be used to express the epigenetic regulator. Expression entails culturing the host cells under conditions suitable for cell growth and expression and recovering the expressed polypeptides from a cell lysate or, if the polypeptides are secreted, from the culture medium. In particular, the culture medium contains appropriate nutrients and growth factors for the host cell employed. The nutrients and growth factors are, in many cases, well known or can be readily determined empirically by those skilled in the art. Suitable culture conditions for mammalian host cells, for instance, are described in Mammalian Cell Culture (Mather ed., Plenum Press 1984) and in Barnes and Sato (1980) Cell 22:649.

In addition, the culture conditions should allow transcription, translation, and protein transport between cellular compartments. Factors that affect these processes are well-known and include, for example, DNA/RNA copy number; factors that stabilize DNA; nutrients, supplements, and transcriptional inducers or repressors present in the culture medium; temperature, pH and osmolality of the culture; and cell density. The adjustment of these factors to promote expression in a particular vector-host cell system is within the level of skill in the art. Principles and practical techniques for maximizing the productivity of in vitro mammalian cell cultures, for example, can be found in Mammalian Cell Biotechnology: a Practical Approach (Butler ed., IRL Press (1991).

Any of a number of well-known techniques for large- or small-scale production of proteins can be employed in expressing a polypeptide of interest. These include, but are not limited to, the use of a shaken flask, a fluidized bed bioreactor, a roller bottle culture system, and a stirred tank bioreactor system. Cell culture can be carried out in a batch, fed-batch, or continuous mode.

Methods for recovery of recombinant proteins produced as described above are well-known and vary depending on the expression system employed. A polypeptide including a signal sequence can be recovered from the culture medium or the periplasm. Polypeptides can also be expressed intracellularly and recovered from cell lysates.

The expressed polypeptides can be purified from culture medium, a cultured cell lysate, or a natural source by any method capable of separating the polypeptide from one or more components of the culture medium, host cell, or natural source. Typically, the polypeptide is separated from components that would interfere with the intended use of the polypeptide. As a first step, the culture medium, cell lysate, or other source material is usually centrifuged or filtered to remove cellular debris. The supernatant is then typically concentrated or diluted to a desired volume or diafiltered into a suitable buffer to condition the preparation for further purification.

The polypeptide can then be further purified using well-known techniques. The technique chosen will vary depending on the properties of the expressed polypeptide. If, for example, the polypeptide is expressed as a fusion protein containing an epitope tag or other affinity domain, purification typically includes the use of an affinity column containing the cognate binding partner. For instance, polypeptides fused with green fluorescent protein, hemagglutinin, or FLAG epitope tags or with hexahistidine or similar metal affinity tags can be purified by fractionation on an affinity column.

Alternatively, the epigenetic regulator can be synthesized by any of a number of widely used techniques, such as for example exclusive solid phase synthesis, partial solid phase synthesis, fragment condensation, and classical solution synthesis. See, e.g., Merrifield, J. Am. Chem. Soc., 85:2149 (1963); John Morrow Stewart and Janis Dillaha Young, Solid Phase Peptide Syntheses (2nd Ed., Pierce Chemical Company, 1984). The non-coding polynucleotide can be produced using standard recombinant or synthetic techniques, as described above.

Isolated complexes according to the invention have a number of uses. Those isolated from cells are useful in conjunction with the method of characterizing characterizing the transcriptional activity of a gene that is a target for an epigenetic regulator, as well as the method of screening for a chromosomal element (CE) for an epigenetic regulator of a target gene. Complexes formed from purified preparations are useful for demonstrating that an epigenetic regulator of interest specifically binds to a particular non-coding polynucleotide.

The isolated complex can include any epigenetic regulator described herein, and the non-coding polynucleotide can correspond to any CE from any target gene for the epigenetic regulator. Complexes according to the invention can be isolated from any cell type described herein.

V. Screening for Modulators of Transcription of Genes that are Targets for Epigenetic Regulators

The role of non-coding polynucleotides in recruiting epigenetic regulators to chromosomal elements (CEs), makes the epigenetic regulator-non-coding polynucleotide-CE interaction an attractive target for use in screening for agents that modulate transcription of genes that are targets for epigenetic regulators. Of particular interest, are screens for agents that modulate transcription of genes that play a role in cell proliferation and/or differentiation, as any agents identified are candidate modulators of these processes.

Accordingly, the invention provides a method of screening for a modulator of transcription of a gene that is a target for an epigenetic regulator. The method is applicable to any target gene that has a cis-regulatory region including a chromosomal element (CE) for the epigenetic regulator, wherein the CE includes a sequence that is a template for a non-coding polynucleotide. The method entails contacting a test agent with a mixture or cell comprising the non-coding polynucleotide and the CE and/or the epigenetic regulator, and detecting the ability of the test agent to modulate specific binding of the non-coding polynucleotide to the CE and/or the epigenetic regulator. More specifically, the method can be carried out by detecting the ability of the test agent to modulate specific binding of the non-coding polynucleotide to the CE, or by detecting the ability of the test agent to modulate specific binding of the non-coding polynucleotide to the epigenetic regulator, or by detecting both. For example, chromatin immunoprecipitation, as described above, could be used to carry out each type of detection. In preferred embodiments, any specific binding is compared with specific binding in the absence of test agent or in the presence of a lower amount of test agent.

The screening method is applicable to target genes, epigenetic regulators, non-coding polynucleotides, and cell types as described above for the other methods of the invention. Screening accordingly to the invention is generally, although not necessarily, carried out in vitro. Thus, screening assays can be carried out, for example, using purified or partially purified components, in cell lysates, in cultured cells, or in other biological samples. In exemplary embodiments, screening is generally most conveniently accomplished with a simple in vitro binding assay, as described above. In preferred binding assays, one binding partner is immobilized and exposed to the second binding partner (which can be labeled) in the presence or absence of the test agent. The immobilized binding partner is then washed to remove any unbound material and the labeled binding partner is then detected. To prescreen large numbers of test agents, high-throughput assays are generally preferred.

In a preferred embodiment, generally involving the screening of a large number of test agents, the screening method includes the recordation of any test agent that induces a difference in specific binding of the non-coding polynucleotide to the CE and/or the epigenetic regulator in a database of candidate agents that may modulate transcription of the target gene.

The term “database” refers to a means for recording and retrieving information. In preferred embodiments, the database also provides means for sorting and/or searching the stored information. The database can employ any convenient medium including, but not limited to, paper systems, card systems, mechanical systems, electronic systems, optical systems, magnetic systems or combinations thereof. Preferred databases include electronic (e.g. computer-based) databases. Computer systems for use in storage and manipulation of databases are well known to those of skill in the art and include, but are not limited to “personal computer systems,” mainframe systems, distributed nodes on an inter- or intra-net, data or databases stored in specialized hardware (e.g. in microchips), and the like.

In certain embodiments, such as for example, those in which the target gene affects cell proliferation and/or differentiation, the methods of the invention include further study of one or more test agents to determine whether the test agent inhibits or stimulates cell proliferation. The degree of cell proliferation observed in the presence of a test agent is preferably compared with the degree of cell proliferation observed in the absence of the test agent or in the presence of a lower amount of test agent. Cell proliferation assays are well known, and any standard proliferation assay can be employed in the invention. Such assays can be carried out in vivo or in vitro, although in vitro assays are generally preferred. In a commercially available assay, cells are quantified by an MTS (3-(4,5-dimethylthiazol-2-yl)-5-(3-carboxylmethoxyphenyl)-2-(4-sulfophenyl)-2H-tetrazolium, inner salt) conversion assay, where MTS conversion to a formazan is proportional to cell number and can be followed by absorbance at 490 nM (Cell Titer 96 AQueous One Solution Cell Proliferation Assay, Promega, Madison, Wis., USA). Inhibitors of cell proliferation are candidates for use in treating conditions characterized by inappropriate proliferation, such as cancer. Stimulators of cell proliferation are candidates for use in treating conditions where enhanced proliferation is desired, such as non-healing wounds.

In a similar manner, test agent can be assayed for effects on cell differentiation, where systems are available or can be established to assay differentiation. Any test agent that is found to modulate differentiation is a candidate for use in modulating the differentiation of stem cells, for example, to generate desired cells and/or tissues, as described above.

TABLE A

Drosophila Homeotic genes
Includes homeodomain proteins with Lim, Pou and
Pax domains
abdominal A
homeodomain - Antennapedia class
Abdominal B
homeodomain - bithorax complex
achintya
homeodomain transcription factor (TGIF
subclass) - required, along with homeodomain protein
Vismay, for spermatogenesis
Antennapedia
homeodomain - Antennapedia class
apterous
homeodomain - lim domain
araucan
homeodomain Pbx class
aristaless
homeodomain - paired-like
Arrowhead
LIM domains and LIM homeodomain
bagpipe
homeodomain - NK-2 class
BarH1 & BarH2
homeodomain
bicoid
homeodomain
buttonless
homeodomain
caudal
homeodomain
caupolican
homeodomain Pbx class
C15 (common alternative name Clawless)
member of the 93E cluster of homeodomain
proteins - regulates spatial patterning of the tarsus, a
distal portion of the leg -
homolog of vertebrate oncogene Hox11
cut
homeodomain - cut domain
defective proventriculus
homeodomain
Deformed
homeodomain - Antennapedia class
Distal-less
homeodomain
drifter (preferred name: ventral veinless)
homeodomain - pou domain
empty spiracles
homeodomain
engrailed
homeodomain - engrailed class - segment
polarity gene
even-skipped
homeodomain - pair rule gene
extradenticle
homeodomain - Pbx class
extra-extra
a homeodomain transcription factor -
regulates motorneuron cell fate by restricting
expression of Even-skipped and Lim2
eyegone
homeodomain & paired domain (paired box)
eyeless
homeodomain & paired domain (paired box)
fushi tarazu
homeodomain - Antennapedia class - pair
rule gene
gooseberry-proximal (common alternative name:
gooseberry-neuro)
homeodomain - paired domain (paired box)
gooseberry-distal (common alternative name:
gooseberry)
homeodomain - paired domain (paired box)
Goosecoid
homeodomain - paired-like
homothorax
homeodomain - HM domain
intermediate neuroblasts defective
homeodomain protein
invected
homeodomain - engrailed class
Ipou (preferred name: Abnormal chemosensory
jump 6)
homeodomain and POU domain
islet (preferred name: tailup)
homeodomain and LIM domain
labial
homeodomain - Antennapedia class
ladybird early and ladybird late
transcription factors - homeodomain
proteins
Lim1
Lim domain and lim homeodomain
mirror
homeodomain - Pbx class
muscle segment homeobox-1
homeodomain
muscle segment homeobox 2 (preferred name:
tinman)
homeodomain - NK-2 class
NK1 (common alternative name: S59)
homeodomain - NK-1 class
NK2 (preferred name: ventral nervous system
defective)
homeodomain - NK2 class
Nkx6 (alternative name: HGTX)
homeobox, NK decapeptide domain
transcription factor - acts within a subclass of early
born neurons to link
neuronal subtype identity to neuronal
morphology and connectivity
onecut
homeodomain and cut domain
Optix
homeodomain and Six domain
orthodenticle
homeodomain - paired-like
paired
homeodomain - paired domain (paired box)
POU domain protein 1 (common alternative name:
pdm-1)
homeodomain - pou domain
POU domain protein 2 (common alternative name:
pdm-2)
homeodomain - pou domain
proboscipedia
homeodomain - Antennapedia class
prospero
novel homeodomain
PvuII-PstI homology 13
homeodomain transcription expressed in the
developing eye - required for rhabdomere morphogenesis
and proper detection of light
reversed polarity
homeodomain
rough
homeodomain
Rx
homeodomain transcription factor - required
for regulation of genes involved in brain morphogenesis
s59 (preferred name: NK1)
homeodomain - NK-1 class
Sex combs reduced
homeodomain - Antennapedia class
shaven (common alternative name: sparkling)
paired domain and homeodomain (partial) -
Pax2, 5 and 8 homolog
sine oculis
homeodomain
sparkling (preferred name: shaven)
paired domain and homeodomain (partial) -
Pax2, 5 and 8 homolog
tailup (common alternative name: islet)
homeodomain and LIM domain
tinman (common alternative name: NK-4 and msh-
2)
homeodomain - NK-2 class
Ultrabithorax
homeodomain - Antennapedia class
unplugged
homeodomain protein
ventral nervous system defective (common
alternative name: vnd or NK2)
homeodomain - NK-2 class
ventral veinless (common alternative name:
drifter)
homeodomain - pou domain
vismay
homeodomain transcription factor (TGIF
subclass) - required, along with homeodomain protein
Achintya, for spermatogenesis
zerknüllt
homeodomain - Antennapedia class - DV
polarity
Zn finger homeodomain 1
zinc finger domain and homeodomain protein -
mutation results in various degrees of local errors in
mesodermal cell fate or positioning
Zn finger homeodomain 2
transcription factor - zinc finger domain
and homeodomain - required for correct proximal wing
development

TABLE B

| Vertebrate Hox gene clusters | | |
|---|---|---|
| HoxA | | |
| HoxB | | |
| HoxC | | |
| HocD | | |
| Trithorax group | | |
| absent, small, or homeotic discs 1 | | |
| absent, small, or homeotic discs 2 | | |
| brahma | | |
| eyelid (also known as osa) | | |
| ISWI | | |
| kismet | | |
| lola like | | |
| modifier of mdg4 | | |
| moira | | |
| Snf5-related 1 | | |
| trithorax | | |
| Trithorax like | | |
| zeste | | |
| fs(1)h | | female sterile (1) homeotic 2xBromo domains |
| z | zeste | DNA binding domain |
| mo | moira | Similar to SWI3 and BAF155/177; - |
| | | probably in complex with Brahma. |
| osa | osa | Allelic to eld, (eyelid). Has ARID |
| | | domain, also found in SWI1. Osa may be part of the |
| | | Brahma complex. |
| lawc | | leg-arista-wing complex |
| | | Genetically characterised as trx-G gene in |
| | | Zorin et al. 1999; Genetics 152: 1045-1055. $$Not yet |
| | | cloned. |

| Polycomb group | |
|---|---|
| Pc | Polycomb Chromo-domain (see Aasland et al. |
| | 1995) |
| ph | polyhomeotic Zinc finger, SAM/SPM domain. |
| | at it's C-terminus. |
| Scm | Sex comb on midleg Similar to ph: 3 |
| | zinc fingers, 2 mbt-domains and a SAM/SPM domain. |
| E(z) | Enhancer of zeste SET-domain Cys/His- |
| | cluster (=SAC domain) |
| see also my E(z) web-pages | |
| Pcl | Polycomblike 2x PHD fingers |
| Psc | Posterior sex combs RING finger, BSP- |
| | domain |
| esc | extra sex combs WD (WD40) repeats |
| mxc | multi sex combs |
| crm | cramped interacts with PCNA. |
| (See also: Gehring's www page) | |
| Sce Sex combs extra | |
| Asx | Additional sex combs |
| pho | pleiohomeotic |
| E(Pc) | Enhancer of Polycomb |
| sxc | super sex combs |

| Su(z)2(D) | |
|---|---|
| Suppressor of zeste 2 | |
| additional sex combs | |
| cramped | |
| enhancer of zeste | |
| Enhancer of Polycomb | |
| extra sexcombs | |
| pipsqueak | |
| pleiohomeotic | |
| PRC1 complex of Polycomb group proteins | |
| Polycomb | |
| polyhomeotic distal | |
| polyhomeotic proximal | |
| Sex combs on midleg | |
| Posterior sexcombs | |
| RING | |
| Esc-E(z) complex of Polycomb group proteins | |
| Chromatin assembly factor 1 subunit | |
| enhancer of zeste | |
| extra sexcombs | |
| Su(z)12 - the histone methyltransferase | |
| activity of the Esc-E(z) complex | |
| Brahma complex of trithorax group proteins | |
| brahma | |
| Brahma associated protein 60 kD | |
| dalao | |
| domino | |
| Enhancer of bithorax | |
| eyelid (also known as osa) | |
| ISWI | |
| moira | |
| Nucleosome remodeling factor - 38 kD | |
| Snf5-related 1 | |
| Enhancers and suppressors of position effect | |
| variegation | |
| cramped | |
| Domina | |
| Enhancer of Polycomb | |
| Enhancer of zeste | |
| Minute (2) 21AB also known as S- | |
| adenosylmethionine synthetase - FlyBase ID: FBgn0005278 | |
| modifier of mdg4 | |
| modulo | |
| mutagen-sensitive 209 also known as | |
| Proliferating Cell Nuclear Antigen | |
| Protein phosphatase 1 at 87B also known | |
| as Su(var)3-6 - FlyBase ID: FBgn0004103 | |
| RNA on the X-1 | |
| Rpd3 | |
| Sir2 | |
| Su(var)205 also known as HP1 | |
| Su(var)3-7 - FlyBase ID: FBgn0003598 | |
| Su(var)3-9 | |
| suppressor of Hairy wing | |
| PEV (included due to their close relationship | |
| to Pc-G and trx-G proteins) | |
| Su(z)2 | Ring-finger, BSP-domain |
| Su(var)3-7 | 5x Cys2His2-fingers |
| Su(var)3-9 | Chromo-domain, SET-domain, |
| | CysHis-cluster |
| E(var)93D | POZ-domain |
| Su(var)2-5 | = DmHP1, Chromo- and Chromo- |
| | Shadow domains, (see Aasland et al. 1995) |
| modulo | 4x RNP |
| Su(var)231 | DNA-binding ??? Cytoskeleton- |
| | associated ??? |
| Su(var)3-6 | Protein phosphatase 1 |
| Su(var)2-1 | |
| Su(var)2-10 | |
| Su(var)3-3 | |

TABLE C

Drosophila SET-domain proteins
Description CG8887-PA.
Species Drosophila melanogaster
Description CG1868-PB.
Species Drosophila melanogaster
Description EG:63B12.3 protein.
Species Drosophila melanogaster
Description CG18136-PA.
Species Drosophila melanogaster
Description AT24727p (CG14590-PA).
Species Drosophila melanogaster
Hypothetical protein CG32799 in chromosome X.
Species Drosophila melanogaster
CG5249-PA (RE26660p).
Species Drosophila melanogaster
AT13626p.
Species Drosophila melanogaster
Q9N6U1
Description Putative heterochromatin protein.
CG3848-PD.
Species Drosophila melanogaster
CG30426-PA.
EG:115C2.10 protein.
CG4565-PA.
SUV9_DROME
CG1716-PA.
CG8651-PA (Cg8651-pd).
Species Drosophila melanogaster
TRX_DROME
Description Trithorax protein.
MES4_DROME
CG8503-PA (GH11294p).
CG6476-PA
Description Eukaryotic translation initiation
factor 2 gamma
ASH1.
Species Drosophila melanogaster
CG8378-PA (BcDNA.LD29892).
CG9640-PA.
CG1868-PA (LD26240p).
CG12119-PA.
CG8651-PB (Cg8651-pc).
Species Drosophila melanogaster
GM10003p.
CG9642-PA.
LD36415p.
Species Drosophila melanogaster
LD39445p.
Species Drosophila melanogaster
CG14122-PA (RE32936p).
Species Drosophila melanogaster
CG11160-PB.
Species Drosophila melanogaster
Domain architecture invented in cellular
organisms
RE62495p.
Species Drosophila melanogaster
LD10743p (CG2995-PA).
Species Drosophila melanogaster
CG40351-PA.3 (Cg40351-pb.3).
Species Drosophila melanogaster
CG17086-PA (RE12806p).
RE75113p.
EZ_DROME
Description Polycomb protein E(z) (Enhancer
of zeste protein).
SD01656p.
Species Drosophila melanogaster
SET8_DROME
Description Histone-lysine N-
methyltransferase, H4 lysine-20 specific (EC 2.1.1.43)
(Histone H4-K20 methyltransferase) (H4-K20-HMTase)
(dSET8).
Species Drosophila melanogaster
CG13363-PA.
Species Drosophila melanogaster
AT13877p (Fragment).
Species Drosophila melanogaster
CG9007-PA.
Species Drosophila melanogaster
RE25548p.
Species Drosophila melanogaster
Domain architecture invented in cellular
organisms
LD31569p.
Species Drosophila melanogaster
Domain architecture invented in cellular
organisms
EG:BACR37P7.2 protein.
Species Drosophila melanogaster
CG11160-PA.
Species Drosophila melanogaster
CG3848-PC.
Species Drosophila melanogaster
SD13650p.
Species Drosophila melanogaster

TABLE D

Human SET-domain proteins
Protein ENSP00000263765
Description PR-domain protein 11.
Species Homo sapiens
ENSP00000325014
Description SET and MYND domain containing
protein 2 (HSKM-B).
Q5W0A7_HUMAN
Description OTTHUMP00000040938 (SET domain,
bifurcated 2).
Q5T715_HUMAN
Description Ash1 (Absent, small, or
homeotic)-like (Drosophila) (Fragment).
Species Homo sapiens
ENSP00000346516
Description Zinc finger protein HRX (ALL-1)
(Trithorax-like protein).
Q96FI6
Description Enhancer of zeste 2, isoform a.
Species Homo sapiens
Domain architecture invented in Coelomata
PRD12_HUMAN
Description PR-domain zinc finger protein 12.
Species Homo sapiens
ENSP00000353218
Description Myeloid/lymphoid or mixed-lineage
leukemia protein 3 homolog (Histone-lysine N-
methyltransferase, H3 lysine-4 specific MLL3) (EC
2.1.1.43) (Homologous to ALR protein).
Species Homo sapiens
ENSP00000326477
Description Probable histone-lysine N-
methyltransferase, H3 lysine-9 specific (EC 2.1.1.43)
(Histone H3-K9 methyltransferase) (H3-K9-HMTase) (SET
domain bifurcated 2) (Chronic lymphocytic leukemia
deletion region gene 8 protein).
Species Homo sapiens
SET07_HUMAN
Description Histone-lysine N-
methyltransferase, H4 lysine-20 specific (EC 2.1.1.43)
(Histone H4-K20 methyltransferase) (H4-K20-HMTase) (SET
domain-containing protein 8) (PR/SET domain-containing
protein 07) (PR/SET07) (PR-Set7).
Species Homo sapiens
Q8IYR2
Description SET and MYND domain containing 4.
Species Homo sapiens
Domain architecture invented in cellular
organisms
ENSP00000343209
Description Nuclear receptor binding SET
domain containing protein 1 (NR-binding SET domain
containing protein) (Androgen receptor-associated
coregulator 267).
Species Homo sapiens
Due to overlapping domains, there are 2
representations of the protein
HRX_HUMAN
Description Zinc finger protein HRX (ALL-1)
(Trithorax-like protein).
Species Homo sapiens
Q96PV2
Description KIAA1936 protein (Fragment).
Species Homo sapiens
Q7Z6T6
Description DJ134E15.1.3 (PR domain
containing 1, with ZNF domain (BLIMP1, PRDI-BF1, B-
lymphocyte-induced maturation protein 1), variant 3)
(Fragment).
Species Homo sapiens
Domain architecture invented in cellular
organisms
Q7Z6T5
Description DJ134E15.1.1 (PR domain
containing 1, with ZNF domain (BLIMP1, PRDI-BF1, B-
lymphocyte-induced maturation protein 1), variant 1)
(Fragment).
Species Homo sapiens
ENSP00000313983
Description WHSC1L1 protein isoform short
Species Homo sapiens
Due to overlapping domains, there are 4
representations of the protein
Q9C0A6
Description KIAA1757 protein (Fragment).
Species Homo sapiens
Q9NR48
Description ASH1.
Species Homo sapiens
Q5QGN2_HUMAN
Description HSPC069 isoform b.
Species Homo sapiens
Domain architecture invented in Eukaryota
Q75MP9
Description Hypothetical protein EZH2
(Fragment).
Species Homo sapiens
Q7Z6T7
Description DJ134E15.1.2 (PR domain
containing 1, with ZNF domain (BLIMP1, PRDI-BF1, B-
lymphocyte-induced maturation protein 1), variant 2)
(Fragment).
Species Homo sapiens
Q9BRZ6
Description SUV420H2 protein.
Species Homo sapiens
ENSP00000223193
Description Enhancer of zeste homolog 2 (ENX-
1).
Species Homo sapiens
Domain architecture invented in Coelomata
ENSP00000261364
Description PR-domain zinc finger protein 6
(Fragment).
Species Homo sapiens
BAA83042
Description KIAA1090 protein (Fragment).
Species Homo sapiens
Due to overlapping domains, there are 2
representations of the protein
Q6GMV2
Description SMYD family member 5.
Species Homo sapiens
Domain architecture invented in cellular
organisms
BAC85636
Description CDNA FLJ41529 fis, clone
BRTHA2014792, weakly similar to ENHANCER OF ZESTE.
Species Homo sapiens
AAQ04808
Description Hypothetical protein FP13812.
Species Homo sapiens
Q5VU52_HUMAN
Description PR domain containing 16.
Species Homo sapiens
ENSP00000352262
Description Zinc finger protein HRX (ALL-1)
(Trithorax-like protein).
Species Homo sapiens
PRD11_HUMAN
Description PR-domain protein 11.
Species Homo sapiens
Domain architecture invented in cellular
organisms
MLL4_HUMAN
Description Myeloid/lymphoid or mixed-lineage
leukemia protein 4 (Trithorax homolog 2).
Species Homo sapiens
Q8IZU7
Description Zinc finger transcription factor
(Fragment).
Species Homo sapiens
ENSP00000229735
Description Histone-lysine N-
methyltransferase, H3 lysine-9 specific 3 (EC 2.1.1.43)
(Histone H3-K9 methyltransferase 3) (H3-K9-HMTase 3)
(HLA-B associated transcript 8) (G9a) (NG36).
Species Homo sapiens
Q9NZW9
Description HSPC069.
Species Homo sapiens
PRDM4_HUMAN
Description PR-domain zinc finger protein 4.
Species Homo sapiens
Q8ND06
Description Hypothetical protein
DKFZp434E1831 (Fragment).
Species Homo sapiens
Q5VUM0_HUMAN
Description PR domain containing 2, with ZNF
domain.
Species Homo sapiens
Q5T4E9_HUMAN
Description PR domain containing 1, with ZNF
domain.
Species Homo sapiens
Q9UPS6
Description KIAA1076 protein (Fragment).
Species Homo sapiens
Q5T714_HUMAN
Description OTTHUMP00000060031.
Species Homo sapiens
Q9BYU9
Description Putative chromatin modulator.
Species Homo sapiens
Due to overlapping domains, there are 4
representations of the protein
ENSP00000339764
Description PR-domain protein 8.
Species Homo sapiens
Q6NXF8
Description PRDM15 protein.
Species Homo sapiens
ENSP00000295833
Description SET and MYND domain containing
protein 1.
Species Homo sapiens
Q658W0
Description Hypothetical protein
DKFZp666P0310.
Species Homo sapiens
PRD16_HUMAN
Description PR-domain zinc finger protein 16
(Transcription factor MEL1).
PRDM1_HUMAN
Description PR-domain zinc finger protein 1
(Beta-interferon gene positive-regulatory domain I
binding factor) (BLIMP-1) (Positive regulatory domain I-
binding factor 1) (PRDI-binding factor-1) (PRDI-BF1).
Species Homo sapiens
Q5VSH9_HUMAN
Description OTTHUMP00000061067.
Species Homo sapiens
BAA06689
Description KIAA0067 protein (Fragment).
Species Homo sapiens
EHMT2_HUMAN
Description Histone-lysine N-
methyltransferase, H3 lysine-9 specific 3 (EC 2.1.1.43)
(Histone H3-K9 methyltransferase 3) (H3-K9-HMTase 3)
(Euchromatic histone-lysine N-methyltransferase 2) (HLA-
B associated transcript 8) (G9a protein) (Protein NG36).
Species Homo sapiens
SET1_HUMAN
Description Histone-lysine N-
methyltransferase, H3 lysine-4 specific SET1 (EC
2.1.1.43) (Set1/Ash2 histone methyltransferase complex
subunit SET1) (SET-domain-containing protein 1).
Species Homo sapiens
Domain architecture invented in Bilateria
ENSP00000264646
Description Enhancer of zeste homolog 1 (ENX-
2).
Species Homo sapiens
O95038
Description Hypothetical protein MLL5
(Fragment).
Species Homo sapiens
Q75MQ0
Description Hypothetical protein EZH2
(Fragment).
Species Homo sapiens
ENSP00000219315
Description no description
Q5VU53_HUMAN
Description PR domain containing 16.
Species Homo sapiens
Q8N9F1
Description Hypothetical protein FLJ37473.
Species Homo sapiens
ENSP00000305899
Description suppressor of variegation 4-20
homolog 1 isoform 2
Species Homo sapiens
Domain architecture invented in cellular
organisms
Protein PRD14_HUMAN
Description PR-domain zinc finger protein 14.
Species Homo sapiens
Domain architecture invented in Coelomata
PRDM7_HUMAN
Description PR-domain zinc finger protein 7.
Species Homo sapiens
AAH39197
Description Similar to ALR protein
(Fragment).
Species Homo sapiens
Q13558
Description NN8-4AG (Fragment).
Species Homo sapiens
Domain architecture invented in cellular
organisms
ENSP00000269844
Description PR-domain zinc finger protein 15
(Zinc finger protein 298).
Species Homo sapiens
EHMT1_HUMAN
Description Histone-lysine N-
methyltransferase, H3 lysine-9 specific 5 (EC 2.1.1.43)
(Histone H3-K9 methyltransferase 5) (H3-K9-HMTase 5)
(Euchromatic histone-lysine N-methyltransferase 1) (Eu-
HMTase1) (G9a-like protein 1) (GLP1).
Species Homo sapiens
Protein Q5VU54_HUMAN
Description OTTHUMP00000044147 (PR domain
containing 16).
Species Homo sapiens
ENSP00000270722
Description PR-domain zinc finger protein 16
(Transcription factor MEL1).
Species Homo sapiens
ENSP00000264808
Description PR-domain zinc finger protein 5.
Species Homo sapiens
NSD1_HUMAN
Description Histone-lysine N-
methyltransferase, H3 lysine-36 and H4 lysine-20
specific (EC 2.1.1.43) (H3-K36-HMTase) (H4-K20-HMTase)
(Nuclear receptor binding SET domain containing protein
1) (NR-binding SET domain containing protein) (Androgen
receptor-associated coregulator 267).
Species Homo sapiens
Due to overlapping domains, there are 2
representations of the protein
ENSP00000296682
Description PR-domain zinc finger protein 9.
Species Homo sapiens
ENSP00000347325
Description Myeloid/lymphoid or mixed-lineage
leukemia protein 3 homolog (Histone-lysine N-
methyltransferase, H3 lysine-4 specific MLL3) (EC
2.1.1.43) (Homologous to ALR protein).
Species Homo sapiens
Q9BYU8
Description Putative Chromatin modulator.
Species Homo sapiens
Due to overlapping domains, there are 4
representations of the protein
PRDM2_HUMAN
Description PR-domain zinc finger protein 2
(Retinoblastoma protein-interacting zinc-finger protein)
(Zinc finger protein RIZ) (MTE-binding protein) (MTB-ZF)
(GATA-3 binding protein G3B).
Species Homo sapiens
Q9H6B5
Description Hypothetical protein FLJ22413.
Species Homo sapiens
SMYD1_HUMAN
Description SET and MYND domain containing
protein 1.
Species Homo sapiens
Q9BZB4
Description IL-5 promoter REII-region-binding
protein.
Species Homo sapiens
ENSP00000271640
Description Histone-lysine N-
methyltransferase, H3 lysine-9 specific 4 (EC 2.1.1.43)
(Histone H3-K9 methyltransferase 4) (H3-K9-HMTase 4)
(SET domain bifurcated 1) (ERG-associated protein with
SET domain) (ESET).
Species Homo sapiens
EZH1_HUMAN
Description Enhancer of zeste homolog 1 (ENX-
2).
Species Homo sapiens
ENSP00000348424
Description PR-domain protein 11.
ENSP00000332995
Description Histone-lysine N-
methyltransferase, H4 lysine-20 specific (EC 2.1.1.43)
(Histone H4-K20 methyltransferase) (H4-K20-HMTase) (SET
domain-containing protein 8) (PR/SET domain-containing
protein 07) (PR/SET07) (PR-Set7).
Species Homo sapiens
Q5T4F9_HUMAN
Description OTTHUMP00000044259 (SET and MYND
domain containing 3).
Species Homo sapiens
Domain architecture invented in cellular
organisms
SUV92_HUMAN
Description Histone-lysine N-
methyltransferase, H3 lysine-9 specific 2 (EC 2.1.1.43)
(Histone H3-K9 methyltransferase 2) (H3-K9-HMTase 2)
(Suppressor of variegation 3-9 homolog 2) (Su(var)3-9
homolog 2).
Species Homo sapiens
Domain architecture invented in Coelomata
SETB1_HUMAN
Description Histone-lysine N-
methyltransferase, H3 lysine-9 specific 4 (EC 2.1.1.43)
(Histone H3-K9 methyltransferase 4) (H3-K9-HMTase 4)
(SET domain bifurcated 1) (ERG-associated protein with
SET domain) (ESET).
Species Homo sapiens
ENSP00000337976
Description Histone-lysine N-
methyltransferase, H3 lysine-9 specific 1 (EC 2.1.1.43)
(Histone H3-K9 methyltransferase 1) (H3-K9-HMTase 1)
(Suppressor of variegation 3-9 homolog 1) (Su(var)3-9
homolog 1).
Species Homo sapiens
PRD13_HUMAN
Description PR-domain zinc finger protein 13.
Species Homo sapiens
Q659A7
Description Hypothetical protein
DKFZp761J1217 (Fragment).
Species Homo sapiens
ENSP00000342720
Description 37 kDa protein
Species Homo sapiens
Domain architecture invented in cellular
organisms
ENSP00000352819
Description PR-domain zinc finger protein 13.
Species Homo sapiens
Domain architecture invented in Deuterostomia
Q86W83
Description SET8 protein.
Species Homo sapiens
ENSP00000259865
Description Histone-lysine N-
methyltransferase, H3 lysine-9 specific 3 (EC 2.1.1.43)
(Histone H3-K9 methyltransferase 3) (H3-K9-HMTase 3)
(HLA-B associated transcript 8) (G9a) (NG36).
Species Homo sapiens
Domain architecture invented in Euteleostomi
Q9Y393
Description CGI-85 protein.
Species Homo sapiens
Q9NWE7
Description Hypothetical protein FLJ10078.
Species Homo sapiens
ENSP00000333986
Description myeloid/lymphoid or mixed-lineage
leukemia 5
Species Homo sapiens
Q6AI17
Description Hypothetical protein
DKFZp686J18276.
Species Homo sapiens
Q86XX6
Description SMYD2 protein (Fragment).
Species Homo sapiens
Q96DQ7
Description Hypothetical protein FLJ30625.
Species Homo sapiens
Domain architecture invented in Homo sapiens
Due to overlapping domains, there are 2
representations of the protein
AAH65287
Description CGI-85 protein.
Species Homo sapiens
BAA20842
Description KIAA0388 protein (Fragment).
Species Homo sapiens
Protein ENSP00000312352
Description PR-domain zinc finger protein 2
(Retinoblastoma protein-interacting zinc-finger protein)
(Zinc finger protein RIZ) (MTE-binding protein) (MTB-ZF)
(GATA-3 binding protein G3B).
Species Homo sapiens
Q8TBK2
Description FLJ21148 protein.
Species Homo sapiens
SUV91_HUMAN
Description Histone-lysine N-
methyltransferase, H3 lysine-9 specific 1 (EC 2.1.1.43)
(Histone H3-K9 methyltransferase 1) (H3-K9-HMTase 1)
(Suppressor of variegation 3-9 homolog 1) (Su(var)3-9
homolog 1).
Species Homo sapiens
ENSP00000305060
Description SET domain and mariner
transposase fusion gene
Species Homo sapiens
PRDM9_HUMAN
Description PR-domain zinc finger protein 9.
Species Homo sapiens
Domain architecture invented in Homo sapiens
Protein SMYD3_HUMAN
Description SET and MYND domain-containing
protein 3 (EC 2.1.1.43) (Zinc finger MYND domain-
containing protein 1).
Species Homo sapiens
AAQ63624
Description Myeloid/lymphoid or mixed-lineage
leukemia (Trithorax homolog, Drosophila).
Species Homo sapiens
Protein ENSP00000310082
Description no description
Species Homo sapiens
ENSP00000253473
Description PR-domain zinc finger protein 9.
Species Homo sapiens
Domain architecture invented in Deuterostomia
O96028
Description Putative WHSC1 protein (MMSET
type II) (TRX5 protein).
Species Homo sapiens
Due to overlapping domains, there are 4
representations of the protein
Q9H787
Description Hypothetical protein FLJ21148.
Species Homo sapiens
Domain architecture invented in cellular
organisms
ENSP00000331557
Description SET and MYND domain-containing
protein 3 (EC 2.1.1.43) (Zinc finger MYND domain-
containing protein 1).
Species Homo sapiens
ENSP00000353758
Description Myeloid/lymphoid or mixed-lineage
leukemia protein 2 (ALL1-related protein).
Species Homo sapiens
Protein MLL2_HUMAN
Description Myeloid/lymphoid or mixed-lineage
leukemia protein 2 (ALL1-related protein).
Species Homo sapiens
Domain architecture invented in Homo sapiens
Due to overlapping domains, there are 32
representations of the protein
Protein ENSP00000304360
Description SET and MYND domain containing 4
Species Homo sapiens
Domain architecture invented in cellular
organisms
Q8N1P2
Description Hypothetical protein FLJ38050.
Species Homo sapiens
Q5TGC2_HUMAN
Description OTTHUMP00000016900.
Species Homo sapiens
Q6P5Y2
Description Hypothetical protein.
Species Homo sapiens
PRDM6_HUMAN
Description PR-domain zinc finger protein 6
(Fragment).
Species Homo sapiens
EZH2_HUMAN
Description Enhancer of zeste homolog 2 (ENX-
1).
Species Homo sapiens
Domain architecture invented in Coelomata
ENSP00000262189
Description Myeloid/lymphoid or mixed-lineage
leukemia protein 3 homolog (Histone-lysine N-
methyltransferase, H3 lysine-4 specific MLL3) (EC
2.1.1.43) (Homologous to ALR protein).
Species Homo sapiens
Domain architecture invented in Eutheria
Due to overlapping domains, there are 80
representations of the protein
Displaying only first 5, you can also display
representations.
MLL5 (Fragment).
Species Homo sapiens
Domain architecture invented in cellular
organisms
PRD15_HUMAN
Description PR-domain zinc finger protein 15
(Zinc finger protein 298).
Species Homo sapiens
Domain architecture invented in Amniota
ENSP00000347342
Description huntingtin interacting protein B
isoform 2
Species Homo sapiens
Domain architecture invented in Eukaryota
AAH01296
Description MLL5 protein (Fragment).
Species Homo sapiens
SET7_HUMAN
Description Histone-lysine N-
methyltransferase, H3 lysine-4 specific SET7 (EC
2.1.1.43) (Histone H3-K4 methyltransferase) (H3-K4-
HMTase) (SET domain-containing protein 7) (Set9)
(SET7/9).
Species Homo sapiens
Domain architecture invented in cellular
organisms
Protein ENSP00000333556
Description Zinc finger protein HRX (ALL-1)
(Trithorax-like protein).
Species Homo sapiens
ENSP00000282699
Description PR-domain protein 8.
Species Homo sapiens
Q9NS29
Description HDCMC04P.
Species Homo sapiens
PRDM5_HUMAN
Description PR-domain zinc finger protein 5.
Species Homo sapiens
Domain architecture invented in Euteleostomi
Q8IWR5
Description Myeloid/lymphoid or mixed-lineage
leukemia 5.
Species Homo sapiens
Domain architecture invented in Eukaryota
Histone-lysine N-methyltransferase, H3 lysine-9
specific 2 (EC 2.1.1.43) (Histone H3-K9
methyltransferase 2) (H3-K9-HMTase 2) (Suppressor of
variegation 3-9 homolog 2) (Su(var)3-9 homolog 2).
Species Homo sapiens
SETMAR protein.
Species Homo sapiens
Domain architecture invented in Eukaryota
Q9BZ95
Description Hypothetical protein WHSC1L1.
Species Homo sapiens
Domain architecture invented in Homo sapiens
Due to overlapping domains, there are 4
representations of the protein
CAE45854
Description Hypothetical protein
DKFZp686C08112 (Fragment).
Species Homo sapiens
ENSP00000222270
Description Myeloid/lymphoid or mixed-lineage
leukemia protein 4 (Trithorax homolog 2).
Species Homo sapiens
SET domain, bifurcated 2 (Fragment).
Species Homo sapiens
Q9BYW2
Description Huntingtin interacting protein 1.
Species Homo sapiens
ENSP00000298728
Description Histone-lysine N-
methyltransferase, H3 lysine-9 specific 5 (EC 2.1.1.43)
(Histone H3-K9 methyltransferase 5) (H3-K9-HMTase 5)
(Euchromatic histone methyltransferase 1) (Eu-HMTase1)
(G9a-like protein 1) (GLP1).
Species Homo sapiens
ENSP00000262519
Description no description
Species Homo sapiens
Q658U6
Description Hypothetical protein
DKFZp666C163.
Species Homo sapiens
Q86WM7
Description PR domain-containing protein 1
beta.
Species Homo sapiens
Q86Y97
Description Suppressor of variegation 4-20
homolog 2.
Species Homo sapiens
Q8NFF8
Description MLL5.
Species Homo sapiens
Q6AW96
Description Hypothetical protein
DKFZp686A20205.
Species Homo sapiens
PRDM8_HUMAN
Description PR-domain zinc finger protein 8.
Species Homo sapiens
SETB2_HUMAN
Description Probable histone-lysine N-
methyltransferase, H3 lysine-9 specific (EC 2.1.1.43)
(Histone H3-K9 methyltransferase) (H3-K9-HMTase) (SET
domain bifurcated 2) (Chronic lymphocytic leukemia
deletion region gene 8 protein).
Species Homo sapiens
ENSP00000335398
Description myeloid/lymphoid or mixed-lineage
leukemia 5
Species Homo sapiens
ENSP00000257745
Description myeloid/lymphoid or mixed-lineage
leukemia 5
Species Homo sapiens
huntingtin interacting protein B isoform 2
Species Homo sapiens
MLL4 protein.
Species Homo sapiens
Domain architecture invented in cellular
organisms
MG44 protein (Fragment).
Species Homo sapiens
Domain architecture invented in cellular
organisms
SMYD2_HUMAN
Description SET and MYND domain containing
protein 2 (HSKM-B).
Species Homo sapiens
Protein ENSP00000354310
Description Nuclear receptor binding SET
domain containing protein 1 (NR-binding SET domain
containing protein) (Androgen receptor-associated
coregulator 267).
Species Homo sapiens
Due to overlapping domains, there are 2
representations of the protein
MLL3_HUMAN
Description Myeloid/lymphoid or mixed-lineage
leukemia protein 3 homolog (Histone-lysine N-
methyltransferase, H3 lysine-4 specific MLL3) (EC
2.1.1.43) (Homologous to ALR protein).
Species Homo sapiens
Due to overlapping domains, there are 80
representations of the protein
Myeloid/lymphoid or mixed-lineage leukemia
protein 2 (ALL1-related protein).
Species Homo sapiens
Domain architecture invented in Eutheria
Due to overlapping domains, there are 64
representations of the protein
Displaying only first 5, you can also display
representations.
Protein AAH09337
Description MLL4 protein (Fragment).
Species Homo sapiens
PREDICTED: KIAA1076 protein
Species Homo sapiens
ENSP00000327505
Description myeloid/lymphoid or mixed-lineage
leukemia 5
Species Homo sapiens
MLL5.
Species Homo sapiens
Domain architecture invented in Eukaryota
Hypothetical protein FLJ22263.
Species Homo sapiens
Domain architecture invented in Euteleostomi
SMYD family member 5
Species Homo sapiens
Domain architecture invented in cellular
organisms

### EXAMPLES

The following examples are offered to illustrate, but not to limit, the claimed invention.

### Example 1

### Non-Coding RNA Transcripts of Trithorax-Response Elements Recruit the Epigenetic Activator Ash1 to Ultrabithorax

### Summary

Epigenetic mechanisms define cell identity and function by maintaining the expression of homeotic genes. The cis-regulatory regions of homeotic genes contain trithorax response elements (TREs) that are targeted by epigenetic activators and transcribed in a tissue-specific manner. However, the functional importance of TRE transcription in epigenetic activation remained mysterious. The present study shows that the transcripts of 3 TREs located in the Drosophila homeotic gene Ultrabithorax (Ubx) mediate transcription activation by recruiting the epigenetic activator Ash1 to the template TREs. The transcription of the TREs coincides with Ubx transcription and recruitment of Ash1 to TREs in larval imaginal discs. Protein-RNA binding assays indicate that the SET-domain of Ash1 binds all three TRE transcripts. Chromatin immunoprecipitation (XChIP) assays in the presence of RNases reveal that each TRE transcript hybridizes with and recruits Ash1 only to the corresponding TRE. Transgenic transcription of TRE transcripts restores recruitment of Ash1 to Ubx TREs and Ubx expression in Drosophila Schneider cells that lack endogenous TRE transcripts. These results exert a model whereby recruitment of epigenetic activators by non-coding TRE transcripts represents an important mechanism for epigenetic activation of homeotic gene expression and cell fate determination.

### Materials and Methods

Expression Plasmids

Baculovirus expression vectors expressing Flag-epitope tagged Ash1C and Ash1N were constructed by inserting ash1 cDNA fragments into pVLFlag (31). DNA encoding amino acids 1-1001 (Ash1N) or 1619-2218 (Ash1C) were generated by PCR using primer pairs that insert an Nde I restriction site at amino acid position 1 (Ash1N) or a start codon embedded in an Nde I restriction site at amino acid position 1619 (Ash1C). PCR products were cloned into the Nde I and Xho I restriction sites of pVLFlag (9) and the functional integrity of the generated DNA was confirmed by DNA sequencing.

Recombinant baculovirus containing the expression plasmids were generated using “Sapphire Baculovirus DNA positive selection vector” (Orbigen) (9). Baculovirus expressing Flag-Ash1ΔN and Ash1SET have been described (9).

DNA Templates for in vitro Transcription

DNA transcribing TRE1 (+)TRE2(+), and TRE3(+) were generated by PCR and subsequently cloned into pCR-TOPO2. 1 (pCR-TOPOTRE) (Invitrogen). DNA corresponding to TREs was inserted into the Xba I and BamH I restriction sites of pBluescript KS+ (Stratagene). The generated plasmids [pBluescriptTRE(+)] transcribe TRE1(+)TRE2(+), and TRE3(+) under the control of the T7 polymerase promoter. For transcription of anti-sense TRE transcripts, DNA corresponding to TRE-1, TRE-2 and TRE-3 was inserted into the Xho I and BamH I restriction sites of pBluescript KS+ (Stratagene). The resulting plasmids [pBluescriptTRE(−)] transcribe anti-sense TRE transcripts under the control of the T7 promoter.

Plasmids Transcribing TRE RNAs in Drosophila Schneider S2 Cells

Plasmids transcribing TRE1(+), TRE2(+) and TRE3(+) in Drosophila S2 cells were generated by releasing TRE-1, TRE-2 and TRE-3 by Xba I and Sac I restriction enzyme digest from pCR-TOPOTRE, followed by insertion into the corresponding restriction sites of pPAC-PL (41). Releasing the DNA for TRE-1, TRE-2 and TRE-3 with Xba I and BamH I from pCR-TOPOTRE and inserting the DNA fragments into the corresponding restriction sites of pPAC-PL generated the pPAC-PL derivatives transcribing the anti-sense RNA of TRE-1, TRE-2 and TRE-3.

Expression and Purification of Proteins

Flag(M2)-tagged Ash1 derivatives were expressed in Sf9 cells that had been infected with recombinant baculovirus as described (9). Recombinant proteins were immunoaffinity purified as described using Flag(M2)-epitope antibodies coupled to agarose (Sigma) (9). Nuclear extract was prepared as described except that histones were removed by hydroxyapatite chromatography (42,43).

Protein-RNA Interaction Assays

Radiolabeled full-length and truncated TRE1(+), TRE2(+), TRE3(+), and the corresponding anti-sense RNAs were generated by in vitro transcription. [pBluescriptTRE(+)] and [pBluescriptTRE(−)] plasmids were linearized with BamH I and Xho I, respectively, and purified. The linearized plasmids were incubated with T7 polymerase (Roche) polymerase in the presence of 10 μCi ³²PaATP, RNasin (10U) in 20 μl reaction buffer (Roche) at 37° C. for 2 h. Templates were removed by DNase (RNase-free DNase I, Roche) digest and the generated RNA purified by using the Rneasy kit (Qiagen).

In vitro protein-RNA interaction assays were programmed with Flag-beads loaded with 200 ng Ash1 derivatives or Mdu, radiolabeled RNA fragment (100,000 c.p.m., 2 ng), and 0.5 μg/μl competitor RNA (yeast total RNA). Reactions were incubated in 300 μl PBS (137 mM NaCl, 2.7 mM KCl, 10 mM Na₂HPO₄, 2 mM KH₂PO₄.) at RT for 2 h. After incubation, Flag-beads were precipitated by centrifugation and washed 3-6 times with HEMG (100 mM Tris/HCl, pH 8.0, 12.5 mM MgCl₂, 0.1 mM EDTA, 10% Glycerol) containing 500 mM KCl and 3 times with HEMG containing 1 M KCl. Precipitated RNA was purified by using TRIZOL reagent (Invitrogen) according to the manufacturers instructions. Purified RNA was analyzed on 4% TBE/polyacrylamide gels by native polyacrylamide gel-electrophoresis (native PAGE). Precipitated RNA was detected by autoradiography.

For competition experiments, in vitro interaction assays were programmed as described except that unlabeled competitor RNA, DNA and RNA/DNA was added. Double stranded TRE RNA was generated by co-transcription of sense and anti-sense strands. Reaction products were separated on agarose gels. Products corresponding to dsRNA were purified from the gel using the QIAEX II gel extraction kit (Qiagen). TREs were generated by releasing the corresponding DNA from pBluescript TRE plasmids by restriction digest. The reaction products were separated by agarose gels. TREs were purified using the QIAEX II gel extraction kit (Qiagen). RNA/DNA hybrids were generated by First strand RT-PCR. Reaction products were separated on agarose gels and DNA/RNA hybrids were purified by using the QIAEX II gel extraction kit (Qiagen). The concentration of competitor nucleic acids was determined by spectrophotometry. Radiolabeled TRE transcripts and competitor nucleic acids were used at a molar ratio of 1:1, 1:5 or 1:20.

Transfection of S2 Cells

S2 cells were maintained and transfected with plasmid DNA essentially as described except that Cellfectin (Invitrogen) was used as a transfection reagent (44). 1×10⁶ cells were transfected with 1 μg pActinGFP and 4 μg pPAC-PL expressing TRE(1(+), TRE2(+), or TRE3(+) and the corresponding anti-sense RNAs. 60 h after transfection, the transfection efficiency was determined by counting the number of GFP-expressing cells. Transfection assays were performed in duplicates and repeated 5 times.

RT-PCR

RT-PCR was performed as described (15) and used to detect RNA in cells and tissue and RNA immunoprecipitated by XChIP and NChIP. RNA was isolated from 1×10⁶ wild type and transfected S2 cells and 100 wild type or ash1²² mutant imaginal discs. Haltere, wing and 3^{rd} leg imaginal discs were prepared by hand from 3^{rd} instar larvae. The homogeneity of the generated pools of discs was confirmed by visual inspection by at least two different individuals. RNA was isolated with TRIZOL reagent (Invitrogen) according to the manufacturers instructions. Purified RNA pools were digested by RNase-free DNase I (Roche) and re-purified by using TRIZOL. For reverse transcription, 0.5-10 μg of the generated RNA was incubated with 2 U Superscript II (Invitrogen) in the presence of dNTPs, RNasin (22 U, Eppendorf), DTT and random hexamer primers in the supplied reaction buffer at 37 C for 2 h. The reverse transcriptase was inactivated by heat (95 C, 5 min). The generated cDNA pools were used as template for PCR assays that were performed as described. RT-PCR assays detecting actin5C RNA standardized the overall amount of transcripts present in isolated RNA pools. TRE, Ubx, actin5C and control-transcripts were detected using PCR primer listed in Tables 1 and 2. PCR products were electrophoretically separated on 2% TBE agarose containing ethidium bromide and visualized by UV light.

TABLE 1
PCR primers for detection of bxd transcripts

| | | |
|---|---|---|
| PCR Primer | Position (22) | Sequence |

| | | |
|---|---|---|
| TRE-1 | | |
| TRE-1-LEFT | 217111-217134 | CCGGTACACGTTATTCACTTCGAC |
| TRE-1-RIGHT | 217571-217590 | CGGCCCTCCATCAACGCTTC |
| TRE-1-3′ | 217937-217953 | ATGAACAGAAGCAGCAG |
| TRE-2 | | |
| TRE2-LEFT | 218835-218856 | CGGAGCAATTTGTCACCGCAAG |
| TRE2-RIGHT | 219230-219249 | GCTCTCGCTTTACGGCGCAG |
| TRE-2-3′ | 218447-218667 | TTGTTGCATATGCAACCCAAG |
| N1 | | |
| N1-LEFT | 219250-218270 | GATCCGAGCGAGAAGGCTAAC |
| N1-RIGHT | 219631-219650 | GTCCCCTTCTAACAGCCGTG |
| TRE-3 | | |
| TRE3-LEFT | 219731-219754 | CATTGTGCTCGGGCACTGATTGAA |
| TRE3-RIGHT | 220035-220058 | GGCACGCACTAAACCCCA |
| S-1 | | |
| S-1-LEFT | 216380-216401 | GGCGTTCGGATAATTTGGCCTC |
| S-1-RIGHT | 217113-217136 | GCGTCGAAGTGAATAACGTGTACC |
| S-2 | | |
| S-2-LEFT | 217626-217651 | CCGGGCGAGTCAATTAAATCAAATGG |
| S2-RIGHT | 218132-218153 | GAGTTCCGTGATTGGATTGCCC |
| S-3 | | |
| S-3-LEFT | 220119-220143 | CGGCATCGGTTGTTTGTTGTTTCTG |
| S-3-RIGHT | 220596-220616 | CCGCGTCCGCAAAACTAGCAA |

TABLE 2
PCR Primer pairs detecting Drosophila genes

| | | |
|---|---|---|
| PCR Primer | Position | Sequence |

| | | |
|---|---|---|
| actin5C | | |
| actin5C-5′ | 456-476 | CGTTCTGGACTCCGGCGAT |
| | | GG |
| actin5C-3′ | 994-1014 | GTACTTGCGCTCTGGCGGG |
| | | GC |
| Antennapedia (Antp) | | |
| Antp-5′ | 53-73 | CGTACATGGGGGCGGACAT |
| | | GC |
| Antp-3′ | 278-298 | CCTGGGGCATGACCCCGCC |
| | | CA |
| Cdc2 | | |
| Cdc2-5′ | 356-376 | GCCATCGTCGGCGAGTACT |
| | | TC |
| Cdc2-3′ | 526-546 | GGAATACCGGGGTGAACCC |
| | | AG |
| Cyclin A (CycA) | | |
| CycA-5′ | 1082-1102 | CCGAGTTGTCGCTCATGGA |
| | | GG |
| CycA-3′ | 1284-1304 | TCCCGCATGGCCTGCGTGT |
| | | TG |
| Cyclin D (CycD) | | |
| CycD-5′ | 93-113 | GTCCTCACCGGCGATCATT |
| | | CG |
| CycD-3′ | 400-420 | GTCGGTTGCGGGTGGATCG |
| | | GC |
| Cyclin E (CycE) | | |
| CycE-5′ | 99-119 | CGGCAGCGAGCAGGGCAAT |
| | | CT |
| CycE-3′ | 620-640 | GAAGTGGGCACTGGCGCAG |
| | | AC |
| Even-Skipped (Eve) | | |
| Eve-5′ | 550-570 | ATGGCCACCGGAATGCCC |
| | | CC |
| Eve-3′ | 1108-1128 | CGCCTCAGTCTTGTAGGGC |
| | | TT |
| String/cdc25 (stg) | | |
| Stg-5′ | 95-115 | GTGGATCTCGTCGTGCTCG |
| | | CC |
| Stg-3′ | 616-636 | TGCTGGCGGTTCCGGGCGC |
| | | TT |
| Twine (twe) | | |
| Twe-5′ | 133-153 | GCCCGCCTGGATGGCACTC |
| | | CC |
| Twe-3′ | 697-717 | CTCGTATCCGCCCTGGCTT |
| | | CC |
| Ultrabithorax (Ubx) | | |
| Ubx-5′ | 614-634 | CGTTCTGGACTCCGGCGAT |
| | | GG |
| Ubx-3′ | 1153-1172 | GTACTTGCGCTCTGGCGG |
| | | GG |
| Ubx promoter | | |
| Ubx-P-5′ | 241-220 | CCATGATGAATTTCCCGCG |
| | | GC |
| Ubx-P-3′ | + (94-114) | AGCGGTAAAGCGCTGAGG |
| | | GC |
| Stg promoter | | |
| Stg-P-5′ | −333-311 | ATCATATGACTGCGGCCAC |
| | | TACC |
| Stg-P-3′ | + (132-157) | CAGGATCATATGGACTCAG |
| | | TTTTGG |

Rapid Amplification of cDNA Ends (RACE)

The transcription of TREs in imaginal discs and S2 cells and the 5′ and 3′ the corresponding transcripts were detected by RACE using the “FirstChoice® ACE Kit” (Ambion). Total RNA was isolated from 100 3^{rd} leg imaginal discs by using the TRIZOL (Invitrogen) according to the manufacturers instructions. Purified RNA pools were incubated with RNase-free DNase I (Roche) and re-purified by using the RNeasy kit (Quiagen). The 5′ and 3′ ends of TRE transcripts were detected using the experimental strategies provided by the FirstChoice® RLM-RACE Kit (Ambion). Briefly, for the detection of 5′-end of transcripts, RNA was treated with alkaline phosphatase, which removes the phosphate-groups of uncapped transcripts. In the second step, tobacco acid pyrophosphatase removes the cap from full-length nascent transcripts. Third, a RACE primer (5′ RACE adapter) is ligated to phosphorylated, decapped transcripts. Reaction products were reverse transcribed. To detect the 3′-end of transcripts, RNA was reverse transcribed by using the 3′ RACE primer. Generated cDNA pools were purified by using the PCR purification kit (Qiagen). PCR analysis using PCR primers located within the bxd (Table 3) and primer detecting the 5′- or the 3′-RACE adapter (Ambion). The generated PCR products were reamplified in a second, nested PCR using bxd PCR primers and inner 5′- and 3′-RACE primers located within the boundaries of the primary PCR product. Second step PCR products were separated on 2% ethidium bromide agarose gels, purified, and cloned into pCR-TOPO using the TOPO cloning kit (Invitrogen). The identity of cloned DNA fragments was uncovered by DNA sequencing (Genomics Institute, UCR). The position of the TRE transcription units tre1, tre2 and tre3 in the bxd is as follows: tre1: 217080-218029; tre2: 218644-219752; and tre3: 219717-220067 (22).

TABLE 3
RACE PCR primers

| | | |
|---|---|---|
| Primer | Position | Sequence |

| | | |
|---|---|---|
| TRE-1 | | |
| 5′RACE TRE-1 | | |
| TRE1RACE1 | 217131-217148 | TCAGGTCAAACGCGTCG |
| TRE1RACE2 | 217254-217275 | ATTTGTGTAACCGTGTGACGGC |
| 3′RACE TRE-1 | | |
| TRE1POLYRACE1 | 217133-217151 | ACGCGTTTGACCTTGAGGC |
| TRE1POLYRACE2 | 217491-217511 | ACACATCCACAAGCGGACCAG |
| TRE-2 | | |
| 5′RACE TRE-2 | | |
| TRE2-RACE1 | 218898-218920 | TTGCAACATCTATAAAAGGG |
| | | CCG |
| TRE2-RACE2 | 218956-218976 | TTCTTTGACATTTGCCGTCGC |
| 3′RACE TRE-2 | | |
| TRE2-POLYRACE1 | 218995-219013 | AAACACGAATACAAGCCCG |
| TRE2-POLYRACE2 | 219082-219105 | AATGCTACTGCTCTCTAGGCC |
| | | ACG |
| TRE-3 | | |
| 5′RACE TRE-3 | | |
| TRE3-5-RACE1 | 219735-219754 | TTCAATCAGTGCCCGAGCAC |
| TRE3-5-RACE2 | 219795-21981 | TTCGCCTGTTGCCTTGGCG |
| 3′RACE TRE-3 | | |
| TRE3-POLYRACE1 | 219775-219797 | AAGCGGAAAACGAAAGAGAG |
| | | CGC |
| TRE3-POLYRACE2 | 219864-219883 | AGCAAACATGTTGCGAGTGC |

Monoclonal Ash1 Antibodies

Monoclonal antibodies to Ash1 were generated by using an Ash1 peptide (Ash1-P) (amino acids 2203-2217; RKTQQSSSSSTANST) coupled to KLH or ovalbumin.

Rats were immunized subcutaneously and intraperitoneally with a mixture of 50 μg peptide-KLH, 5 nmol CPG oligonucleotide (Tib Molbiol, Berlin), 500 μl PBS and 500 μl immuno Freundsches Adjuvant (IFA) and boosted 6 weeks later omitting IFA. After fusion of the myeloma cell line P3X63 Ag 8.653 with immune rat spleen cells, positive clones were identified with a solid phase enzyme linked immunosorbent assay (ELISA) using Ash1-P-Ovalbumin for coating. On the basis of their reaction pattern, the cell lines Ash1 5D12, 7G12 and 8C1, all of rat IgG1 subclass were established.

In vivo Cross-Linked Chromatin Immunoprecipitation (XChIP)

XChIP was performed essentially as described (9). In vivo cross-linked chromatin was isolated from 2.5×10⁵ wild type or transfected S2 cells or 60-100 imaginal discs per immunoprecipitation. Discs were isolated by hand (9). Cells and discs were incubated with 1.8% formaldehyde for 15 min. The reaction was stopped by incubating the samples in 4 mg/ml glycine for 5 min at RT. In vivo cross-linked chromatin was precipitated and sheared by sonication to an average fragment length of 400 basepairs. To monitor the presence of Ash1, the Ash1 histone modification pattern and TRE transcripts at the TREs and promoter of Ubx and the CEs MCP, iab4 and Fab7 of the bithorax complex (11,22). Chromatin was immunoprecipitated with the following antibodies: tri-methylated H3-K4 (2 Hg/IP, Abcam), tri-methylated H3-K9 (2 μg/IP, Abcam), tri-methylated H4-K20 (2 μg/IP, Abcam), Ash1 (this study), di-methylated H3-K9 (2 μg/IP, UpSTATE, and rabbit and rat serum (10 μg). Chromatin-antibody complexes were purified by Protein-A agarose affinity-chromatography. To purify precipitated DNA chromatin was incubated RNase and Proteinase K to remove RNA and proteins. To purify precipitated RNA chromatin was incubated with DNase (Roche) and Proteinase-K (Roche) to remove DNA and proteins. After enzyme treatment, chromatin was incubated at 65° C. for 6 h to reverse the cross-links. Precipitated DNA and RNA were purified. PCR and RT-PCR detected the presence of precipitated DNA and RNAs, respectively, in generated nucleic acid pools. PCR primer pairs were used to amplify precipitated Ubx, the Ubx promoter, CEs and TRE transcripts. PCR products were analyzed by gel electrophoresis using ethidium bromide containing agarose gels and detected by UV illumination.

Native Chromatin Immunoprecipitation (NChIP)

NChIP was performed as described for XChIP except that native chromatin was used. Native, sheared chromatin was resuspended in PBS and incubated with antibodies to Ash1 or modified histones in the presence of RNase-A (1 mg/ml), RNase-H (1200 U/ml), or RNase-III (650 U/ml) for 12 h at 25° C. Immunoprecipitated DNA and RNA was purified and used as template for PCR and RT-PCR, respectively, detecting TRE transcripts, TREs or CEs.

### Results

The Ubx TREs are Transcribed in Drosophila Imaginal Discs

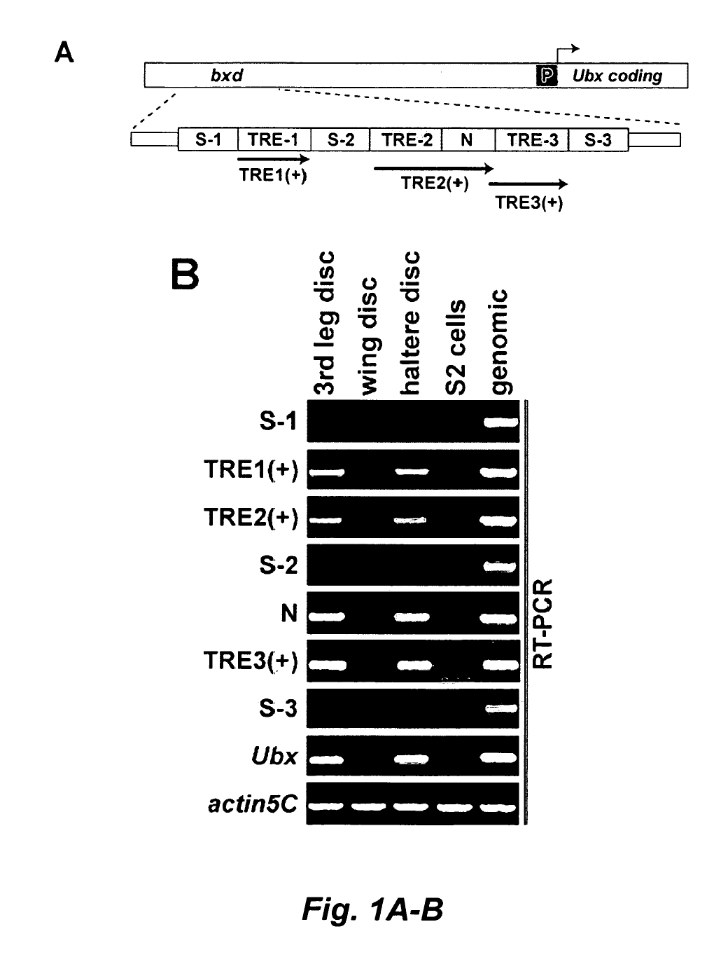

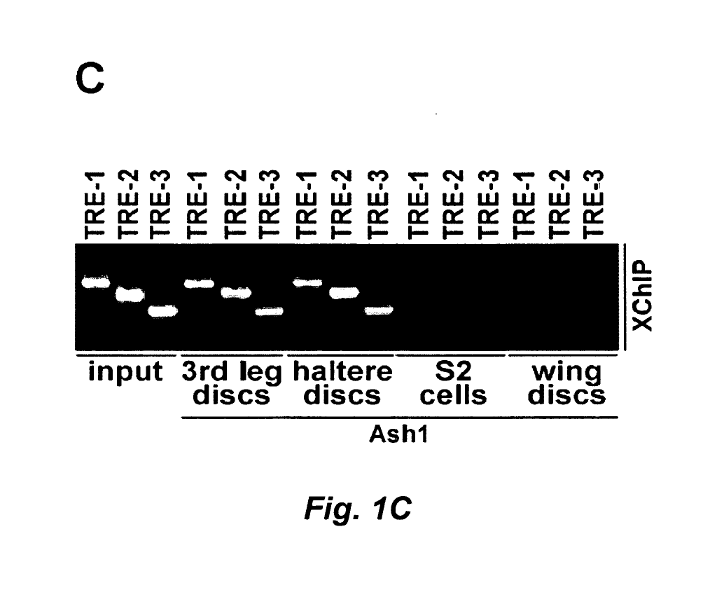

NcRNAs play fundamental roles in various epigenetic phenomena such as gene dosage compensation, imprinting, and silencing (1, 16-19). The resemblance of the tissue-specific transcription and trans-regulatory activity patterns of CEs and trxg proteins, respectively, raised the intriguing possibility that not only transcription of CEs per se but also the resulting ncRNAs might play a functional role in epigenetic activation. To assess the functional importance of CE transcripts for epigenetic activation, the role, if any, of ncRNAs transcribed from the TRE/PREs of Ubx in the recruitment of Ash1 to Ubx was investigated. Ubx expression plays a fundamental role in cell fate determination during Drosophila development (20). For example, Ubx activity is essential for the development of 3rd-leg imaginal discs (3rd-leg discs) and haltere imaginal discs (haltere discs), while repression of Ubx expression is a prerequisite of wing development (10,20). The Ubx locus contains a cluster of 3 characterized PRE/TREs (TRE1, -2, -3) within the boundaries of the chromosomal memory element bxd that is located 22 kb upstream of the Ubx promoter (FIG. 1A) (21, 22). Bxd is transcribed in Drosophila embryos and larvae (12,13,23). In contrast, the transcription status of bxd in leg, haltere, and wing imaginal discs, which represent the sphere of action of Ash1, and the functional relationship, if any, between bxd transcription and Ash1-mediated activation of Ubx transcription remained unknown.

To correlate the transcriptional activity of Ubx with bxd transcription in imaginal discs, bxd transcripts were detected in 3rd-leg discs by using “Rapid Amplification of cDNA ends” (RACE). RNA was isolated from 3rd-leg discs prepared from third-instar larvae. Bxd transcripts were detected by using the FirstChoice® RLM-RACE Kit (Ambion) that detects 5′-capped, and poly-adenylated RNAs. The 5′ and 3′ ends of transcripts were detected by use of specific RACE PCR primers in combination with PCR primers located within bxd. The identity of PCR products was uncovered by DNA sequencing. Three capped, polyadenylated bxd transcripts were detected in 3rd-leg and haltere discs (FIG. 1A). All 3 transcripts are transcribed from the coding strand of bxd with respect to the Ubx transcript. The TRE1(+) transcript (949 nt) originates from a DNA element covering TRE-1 (FIG. 1A). TRE(2⁺) (1108 nt) corresponds to TRE2 and the linker region separating TRE-2 and TRE-3 (N) (FIG. 1A). TRE3(+) (350 nt) is transcribed by a DNA element that contains TRE-3 (FIG. 1 A). Of note, all three transcripts do not contain open reading frames of significant length.

Computational DNA sequence comparison revealed that the transcription of all 3 TRE-derived RNAs (TRE transcripts) is controlled by promoter motifs (TATA-box, initiator region) characteristic to the RNA polymerase II (RNAP-II) transcription machinery (Sanchez-Elsner and Sauer, data not shown). Thus, the data uncovers the existence of three novel transcription units, which were termed tre1, tre2 and tre3, in the bxd of Ubx.

The Transcription of Ubx TREs Coincides with Ubx Transcription in Drosophila

The relationship of the presence of the 3 TRE transcripts to Ubx transcription was examined next. RT-PCR was used to detect the TRE transcripts in cells and tissues that transcribe Ubx or not. RNA was isolated from 3rd-leg discs and haltere imaginal discs (haltere discs), which both transcribe Ubx, and wing imaginal discs (wing discs) and S2 cells that do not (9,11). Isolated RNA pools were subjected to RT-PCR that detected the transcripts of Ubx tre1, tre2, and tre3, and the control, actin5C. Ubx and all 3 TREs were detected in 3rd-leg and haltere discs (FIG. 1B). In contrast, Ubx and TRE transcripts were not detected in S2 cells and wing discs (FIG. 1B). Cumulatively, our results indicate that Ubx expression in 3rd-leg and haltere discs coincides with the presence of TRE transcripts.

Recruitment of Ash1 to Ubx TREs Coincides with the Presence of TRE Transcripts

The 3 Ubx PREs/TREs are targets for several epigenetic regulators, many expressed in a ubiquitous rather than cell type-specific fashion (3,4). The co-transcription of the Ubx TREs and Ubx in 3rd-leg discs raised the possibility that TRE transcription might contribute to the cell type-specific recruitment of epigenetic activators to TREs. To test this hypothesis, the recruitment of the epigenetic activator Ash1 to the Ubx TREs was investigated in 3rd-leg discs, haltere discs, wing discs and S2 cells, which express ash1 (9,10), by in vivo cross-linked chromatin immunoprecipitation (XChIP) (9). In vivo cross-linked chromatin was isolated from cells and discs, sheared into fragments containing 400 bp DNA, on average, and immunoprecipitated with antibodies to Ash1, the Ash1-mediated histone methylation pattern and rat or rabbit anti-serum as a control. Immunoprecipitated DNA was purified and used as a template for PCR assays that detected the presence of the Ubx TREs in precipitated DNA pools.

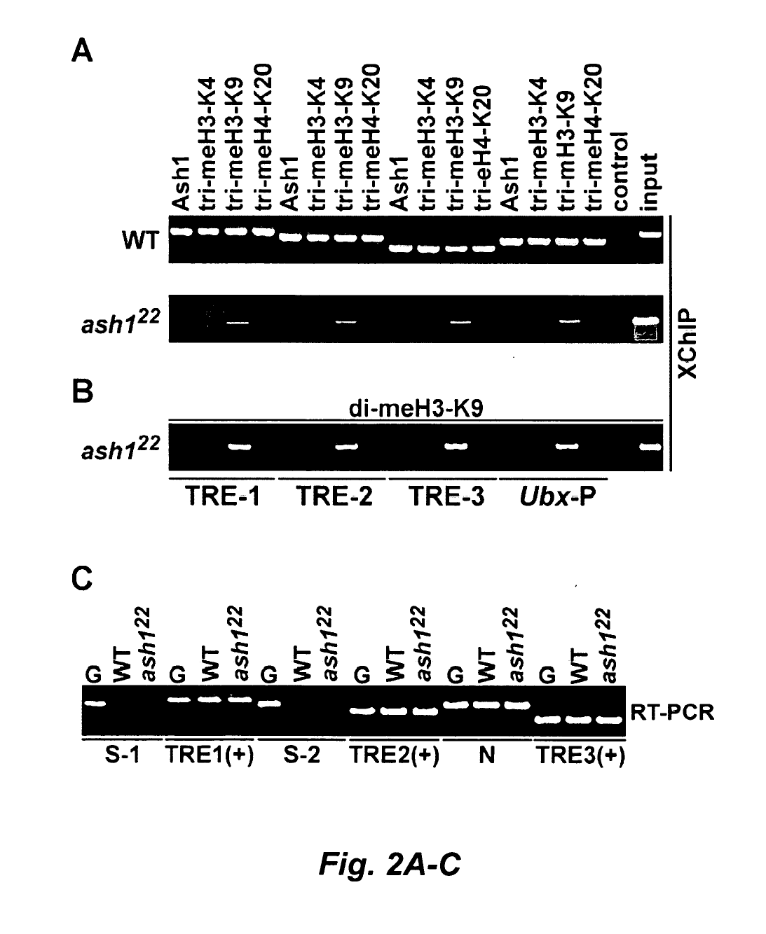

Ash1 was detected at all 3 TREs in 3rd-leg and haltere discs, which contain TREs and transcribe Ubx (FIG. 1C). In addition, the Ash1 histone methylation pattern was detectable in all 3 TREs and the transcriptionally active Ubx promoter in 3rd-leg discs (FIGS. 1C, 2A). Most interestingly, Ash 1 was not detected at the TREs of the transcriptionally inactive Ubx locus in wing discs and S2 cells, which do not transcribe TREs, indicating that the recruitment of Ash1 to the Ubx TREs coincides with the presence of TRE transcripts (FIG. 1C).

To verify this result and the role of Ash1 and Ash 1-mediated histone methylation in Ubx transcription, the recruitment of Ash1 to Ubx was compared in wild type and homozygous mutant ash1²² 3rd-leg discs by XChIP. Ash1²² is recessive lethal and expresses a truncated ash1 protein (amino acids 1-47) that lacks the SET domain and does not activate Ubx transcription (10). XChIP was performed as described, except that in vivo cross-linked chromatin was isolated from homozygous mutant ash1²² 3rd-leg discs. Ash1 and the Ash1 histone methylation pattern were detected at the transcriptionally active Ubx locus in wild-type discs (FIG. 2A). In contrast, in the ash1²² mutant background, Ash1 and the Ash1-mediated histone methylation pattern were undetectable at the TREs and the promoter of Ubx, which indicates that recruitment of Ash1 and Ash1-mediated histone methylation is essential for activation of Ubx expression in 3rd-leg discs (FIG. 2A). Of note, significant levels of di-methylated H3-K9 were detected at the transcriptionally inactive Ubx locus in ash1²² mutant discs (FIG. 2B), indicating that tri-methylation of H3-K9 at the transcriptionally active Ubx locus is mediated by Ash1.

To determine whether Ash1 regulates TRE transcription in 3rd-leg discs, TRE transcription was monitored in the wild type and ash1²² mutant 3rd-leg discs by RT-PCR. TRE transcripts were detected at comparable levels in wild type and mutant discs, which indicates that Ash1 is not a major regulator of TRE transcription in imaginal discs (FIG. 2C). In summary, our data indicate that TRE transcripts play an important role in Ubx transcription and recruitment of Ash1 to Ubx.

The SET-Domain of Ash1 Interacts with TRE Transcripts in vitro

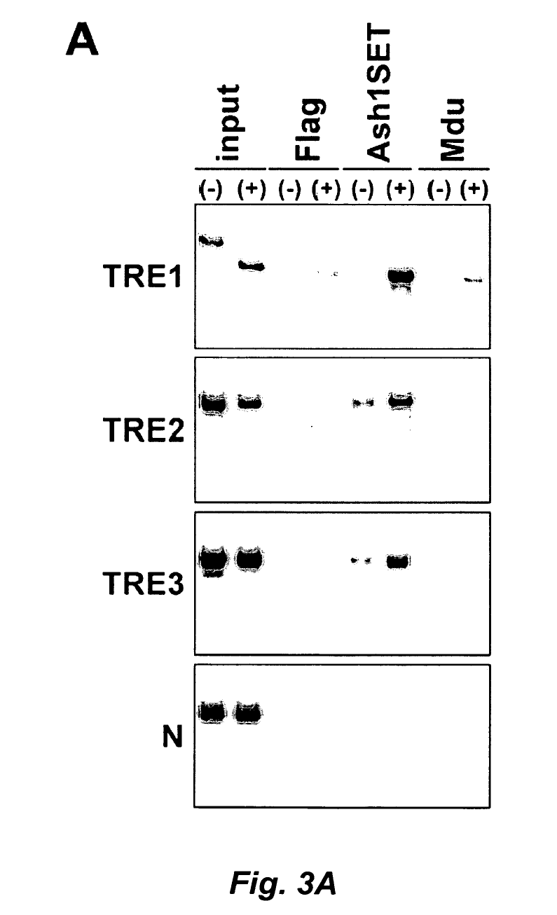

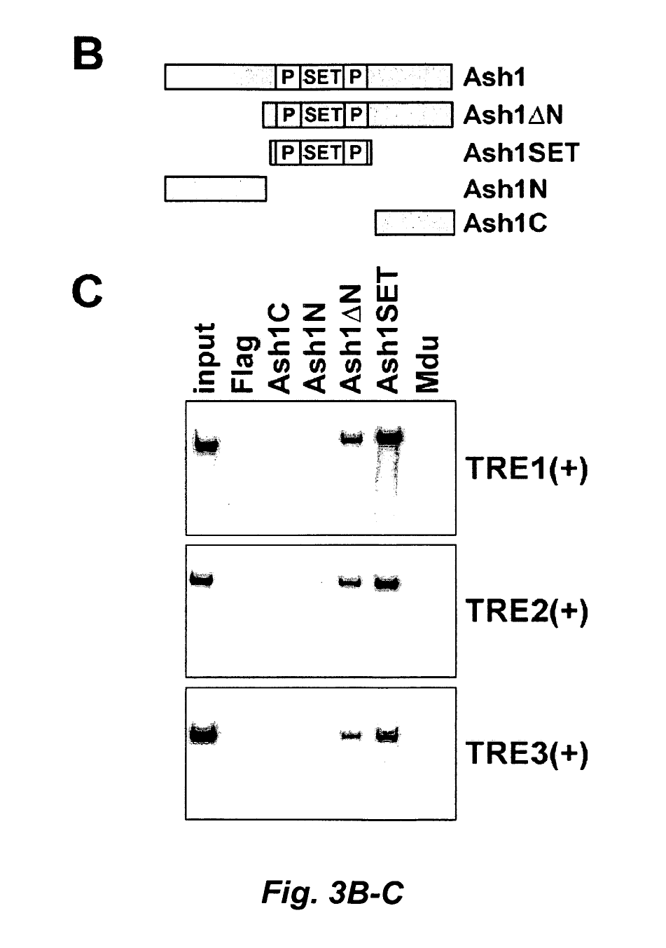

The association of Ash1 with TREs in cells containing TRE transcripts strongly argues for the possibility that transcription of TREs per se or TRE transcripts directly nucleate recruitment of Ash1 to Ubx TREs. The latter hypothesis is consistent with a recent experiment demonstrating that SET-domain proteins can bind single-stranded RNA and DNA in vitro and other studies describing a role of ncRNA in protein recruitment in epigenetic phenomena such as gene dosage compensation (16-19,24). In vitro protein-RNA binding assays were used to assess whether Ash1 associates with TRE transcripts. Radiolabeled full-length and truncated TRE transcripts and, as controls, the complementary, anti-sense RNA of TREs were generated by in vitro transcription. RNA was incubated with anti-Flag antibody agarose resin (Flag-beads) and Flag-beads loaded with recombinant Flag-epitope tagged Ash1 DN, which consists of amino acids 1001-2218 and lacks the NH2-terminal third of the protein, or the H3-K9-specific HMT Medusa (Mdu) (Gou and Sauer data not shown). After incubation, precipitated protein-RNA complexes were washed to remove unbound RNA. Precipitated RNA was purified, separated by native polyacrylamide gel-electrophoresis PAGE and detected by autoradiography. Ash1DN but not Mdu retained TRE1(+), TRE2(+) and TRE3(+) (FIG. 3A). In contrast, Ash1DN and Mdu did not bind the anti-sense RNA of the Ubx TREs, which indicates that Ash1 specifically associates with TRE1(+), TRE2(+) and TRE3(+) (FIG. 3A). Notably, Ash1 did not retain the transcript of the N bxd-element (FIG. 3A), which is an integral part of the TRE2(+) transcript and corresponds to the transcript of the DNA spacer separating TRE-2 and TRE-3 (FIG. 1A). This result indicates that the interaction of Ash1 with TRE transcripts is confined to RNAs corresponding to the described identified TREs (ref. 21).

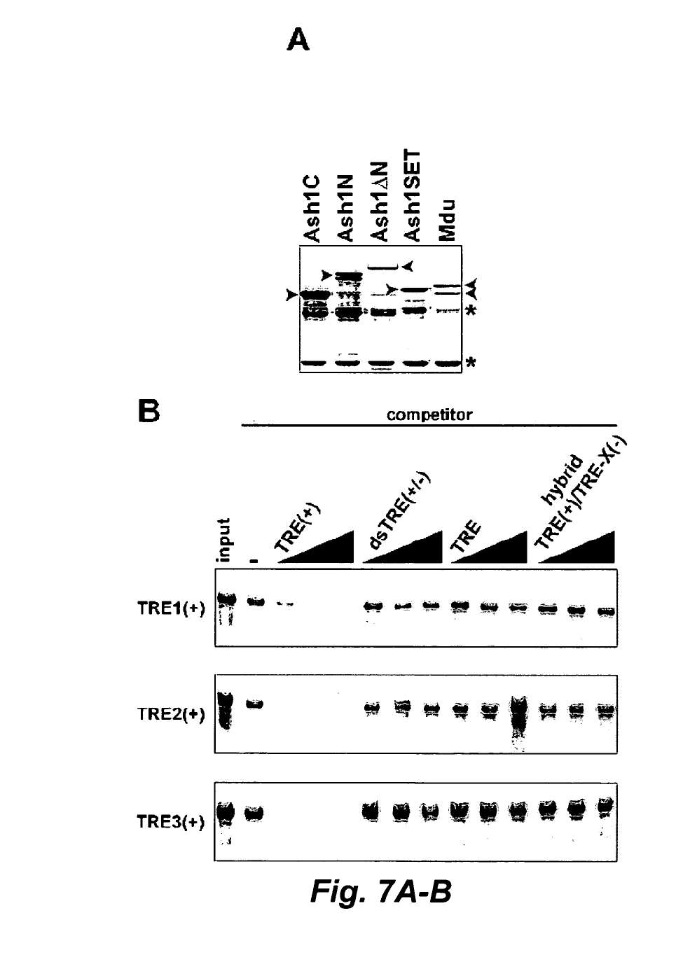

In competition experiments, unlabeled TRE1(+), TRE2(+), and TRE3(+) could compete out the interaction of Ash1 with the corresponding TRE transcript (FIG. 7). In contrast, double stranded TRE transcripts, TREs, and DNA-RNA hybrids comprised of the TRE-transcripts and TREs failed to disrupt the interaction of Ash1 with TRE transcripts, indicating that Ash1 preferentially binds to TRE transcripts (FIG. 7). Most important, the inability of TREs to compete out the interaction of Ash1 with TRE transcripts argues against the possibility that the association of Ash1 with TRE transcripts induces a DNA biding activity in Ash1.

To delineate the RNA-binding motif of Ash1, we investigated the interaction of truncated ash1 proteins with TRE transcripts by in vitro protein-RNA binding assays. In addition to Ash1DN, we tested Ash1 SET (amino acids 1001-1619), which contains the Ash1 SET-module, Ash1N (amino acids 1-1001) and Ash1C (amino acids 1619-2218) (FIG. 3B). Ash1N and Ash1C lack the SET domain and cysteine-rich regions. In protein-RNA binding assays, Ash1DN and Ash1SET but not Ash1N and Ash1C retained TRE1(+), TRE2(+) and TRE3(+), which indicates that the SET-module of Ash1 binds TRE transcripts in vitro (FIG. 3B).

RNA-Dependent Recruitment of Ash1 to Ubx TREs in Drosophila

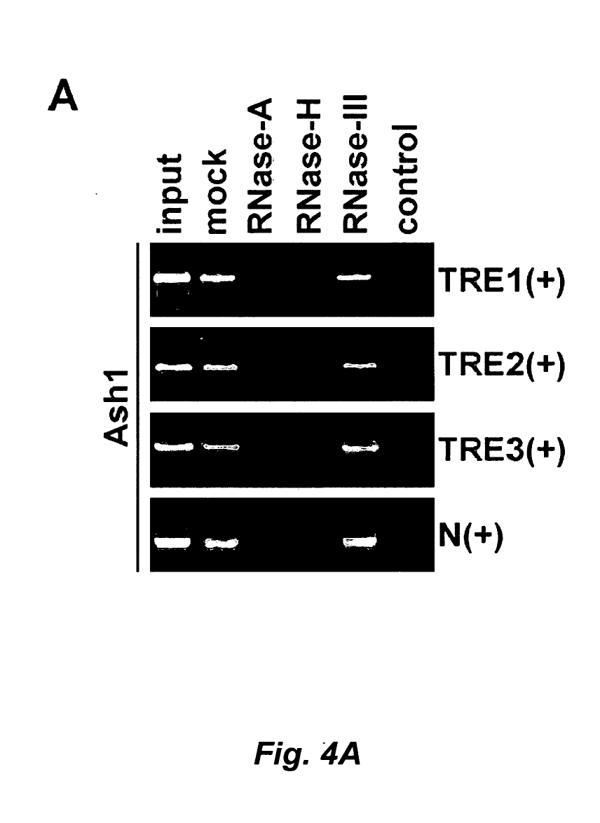

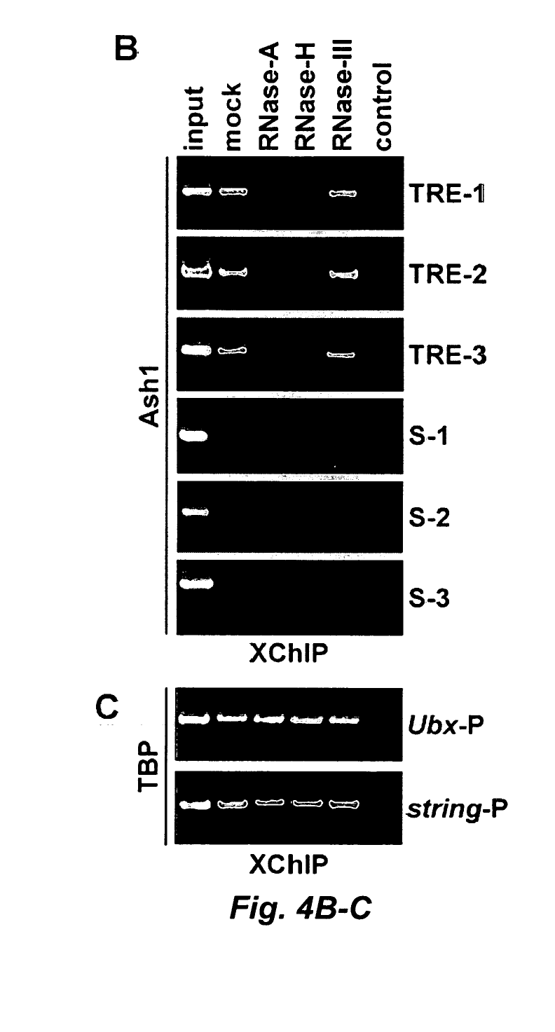

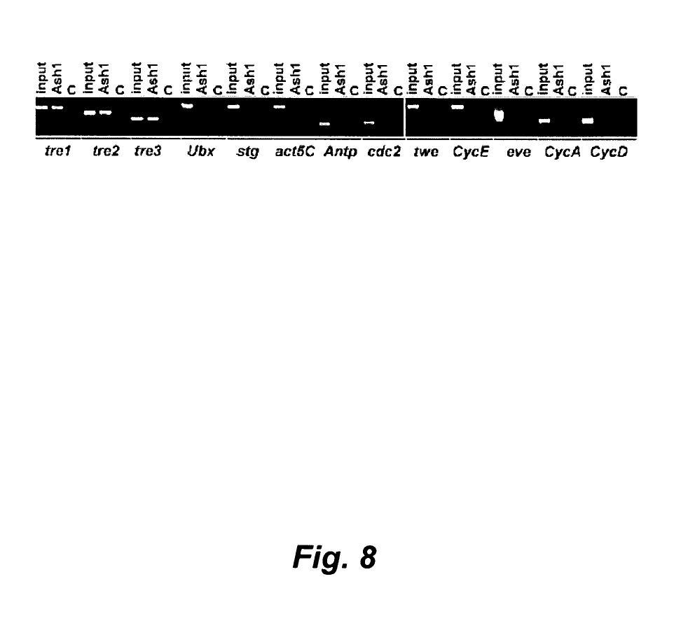

To determine whether Ash1 associates with TRE transcripts in vivo, the question of whether Ash1 co-purified with TRE transcripts from chromatin was investigated using in vivo cross-linked chromatin immunoprecipitation (XChIP) assays. Native chromatin was isolated from 3rd-leg discs, sheared, and incubated with BSA (mock) or different RNases. The RNAases tested were: RNase-A, which degrades single-stranded (ss) RNA; RNase-H, which degrades DNA-RNA hybrids; and RNase-III, which digests double-stranded (ds) RNA. RNase- and mock-treated chromatin was cross-linked using formaldehyde and immunoprecipitated with antibodies to Ash1 and control antibody. Immunoprecipitated RNA was purified and reverse transcribed. RT-PCR detected the presence of TRE transcripts and control transcripts such as Ubx, actin5C, and string/cdc25 (stg) in the generated cDNA pools. Ash1 co-precipitated with TRE transcripts from mock-treated chromatin (FIG. 4A). In contrast, Ash1 did not retain control transcripts (FIG. 8). Ash1 associated with TRE transcripts in RNase-III-treated chromatin, which indicates that TRE transcripts are immune to RNase-III and that double-stranded RNA motifs do not contribute to the association of TRE transcripts with Ash1 in vivo (FIG. 4A). In contrast, Ash1 did not retain TRE transcripts from RNase-A and —H-treated chromatin. (FIG. 4A) Attenuation of Ash1-RNA interactions by RNAse-A indicates that single stranded RNA motifs are important for the association of Ash1 with TRE transcripts. The disruption of the association between Ash1 and TRE transcripts by RNase-H, which disrupts DNA-RNA hybrids, in chromatin provide the first line of evidence that TRE transcripts hybridize with DNA in chromatin. In summary, our results indicate that Ash1 associates with TRE transcripts in chromatin.

Next, it was determined whether the association of Ash1 with TREs is RNA dependent. XChIP was used to compare the interaction of Ash1 and TRE in mock- and RNase-treated chromatin. Chromatin was isolated from 3rd-leg discs, sheared, treated with RNase-A, -H, and -III or BSA (mock), cross-linked, and immunoprecipitated with antibodies to Ash1. PCR detected the presence of TREs and spacer DNA elements (S-1, S-2, and S-3) (FIG. 1A) in precipitated DNA pools.

Antibodies to Ash1 precipitated all 3 TREs but not the spacer DNAs (S1-S3) from mock-treated and RNase-III-treated chromatin, which indicates that dsRNA does not contribute to the interaction of Ash1 with TREs (FIG. 4B). In contrast, treating chromatin with RNase-H or -A attenuated the association of Ash1 with TREs, which indicates that the association of Ash1 with the Ubx TREs is RNA-dependent. (FIG. 4B) The disruption of the interaction of Ash1 with TREs in chromatin by RNase-H and -A raises the hypothesis that single stranded RNA motifs in RNA-DNA hybrids play an essential role in the recruitment of Ash1 to TREs.

To verify that the observed attenuation of Ash1-TRE interactions is based on specific rather than general disruption of protein-DNA interactions in RNase-treated chromatin, the recruitment of the general transcription factor TFIID to target genes was investigated in mock- and RNase-treated chromatin. TFIID consists of the TATA-box binding protein (TBP) and several TBP-associated factors (TAFs) and nucleates transcription initiation by RNAP-II (25). TBP interacts with the TATA-box in promoters and is believed to contribute to the nucleating function of TFIID by tethering TFIID to promoters (25). Mock- and RNase-treated native chromatin from 3rd-leg discs was sheared and immunoprecipitated with antibodies to TBP. PCR detected the interaction of TBP with the promoter of Ubx and stg whose transcription requires TFIID activity (26). TBP interacted with both promoters in mock- and RNase-A-, -H-, and -III-treated chromatin, which indicates that RNase treatment did not attenuate TBP-promoter interactions and protein-gene interactions in general (FIG. 4C). Collectively, the data indicate that the recruitment of Ash1 to the TREs of Ubx is mediated by RNA. Because XChIP detects chemically cross-linked complexes between proteins and nucleic acids, the co-precipitation of Ash1 with TREs and TRE transcripts supports the existence of a trimeric protein nucleic-acid complex in chromatin consisting of Ash1, TREs and TRE transcripts.

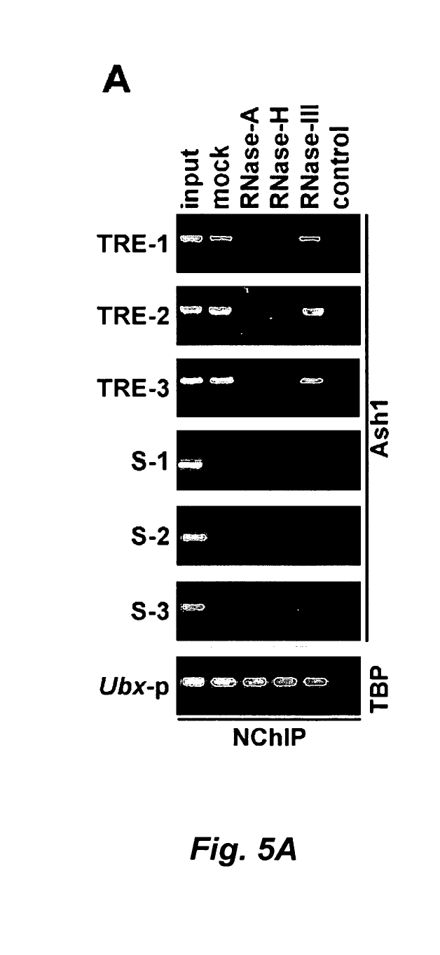

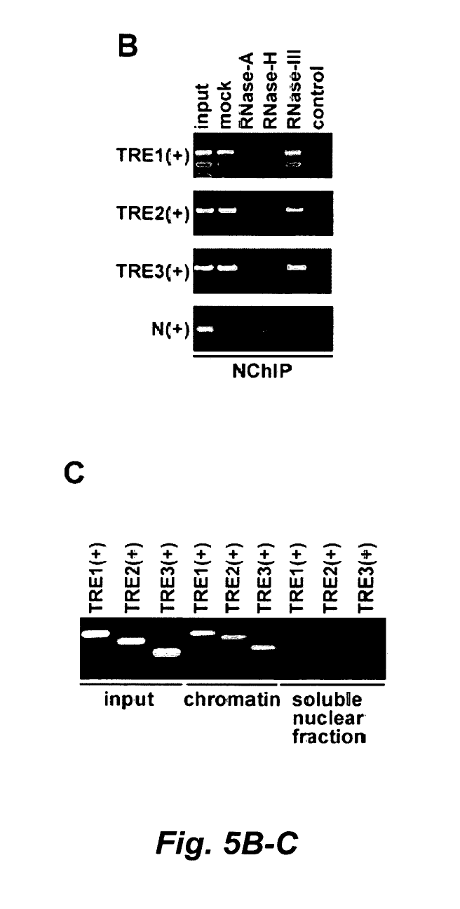

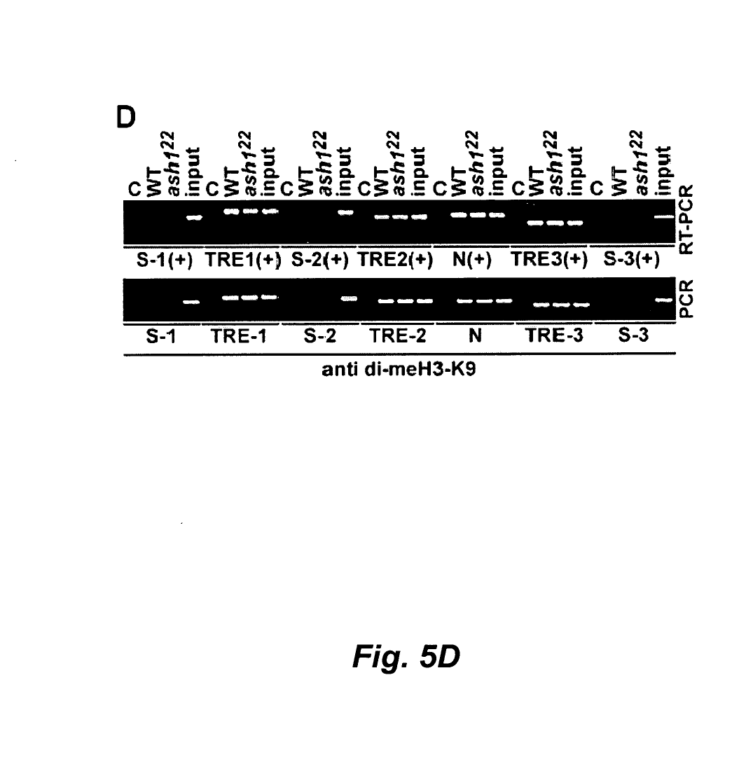

To test whether the detected association of Ash1 with TREs and TRE transcripts occurs in chromatin or is the result of fortuitous interactions generated in chemically cross-linked chromatin, the association of Ash1 with TRE transcripts and TREs was investigated in native chromatin by using native chromatin immunoprecipitation (NChIP). Native chromatin was isolated from 3rd-leg discs, treated with RNase-A, —H, and —III or BSA (mock), sheared, and immunoprecipitated with antibodies to Ash1. Immunoprecipitated chromatin was washed and halved to isolate precipitated DNA and RNA. PCR and RT-PCR detected precipitated TREs and TRE transcripts, respectively. Ash1 was associated with all three TREs and TRE transcripts in mock- and RNase-III-treated chromatin but not RNase-H or -A-treated chromatin, which indicates that Ash1 co-immunoprecipitates with TREs and TRE transcripts in native chromatin (FIG. 5A,B). Most interestingly, an association of Ash1 with the NI portion of the TRE2(+) transcript, as observed in cross-linked chromatin, was not detectable in native chromatin, indicating that, like in vitro, Ash1 binds the RNA corresponding to TRE-2 but not the N region of the TRE2(+) transcript. In summary, the results indicate that Ash1 associates with TRE transcripts and TREs in chromatin and that TRE transcripts interact with chromatin.

Ash1 Associates with Chromatin-Bound TRE Transcripts

To assess whether TRE transcripts are retained at chromatin it was investigated whether Ash1 co-precipitates TRE transcripts from chromatin-free nuclear extract. Ash 1 was immunoprecipitated from nuclear extract and native chromatin prepared from 3rd-leg discs. Ash1 retained TRE transcripts from chromatin but not chromatin-free nuclear extract (FIG. 5C), which indicates that TRE transcripts are preferentially associated with chromatin in the cell.

To determine whether the association of Ash1 with TRE transcripts precedes the recruitment of Ash1 to TREs in chromatin, or, vice versa, Ash1 is recruited to chromatin associated TRE transcripts, XChIP was used to assess whether TRE transcripts are retained at TREs in the absence of Ash1. In vivo cross-linked chromatin was isolated from wild type and ash1²² mutant 3rd-leg discs, sheared and immunoprecipitated with antibodies to di-methylated H3-K9 present at the TREs of the transcriptionally active and inactive Ubx locus in 3rd-leg discs (FIGS. 2A,B). RT-PCR and PCR detected the presence of TRE transcripts and TREs, respectively, in immunoprecipitated RNA- and DNA-pools. The antibody to di-methylated H3-K9 co-precipitated with TREs and TRE transcripts from the chromatin of wild-type and ash1²² 3rd-leg discs (FIG. 5D), indicating that TRE transcripts are retained at Ubx TREs prior to the recruitment of the epigenetic activator Ash1.

TRE Transcripts Restore Recruitment of Ash1 to Ubx TREs and Ubx Transcription in Drosophila Cells

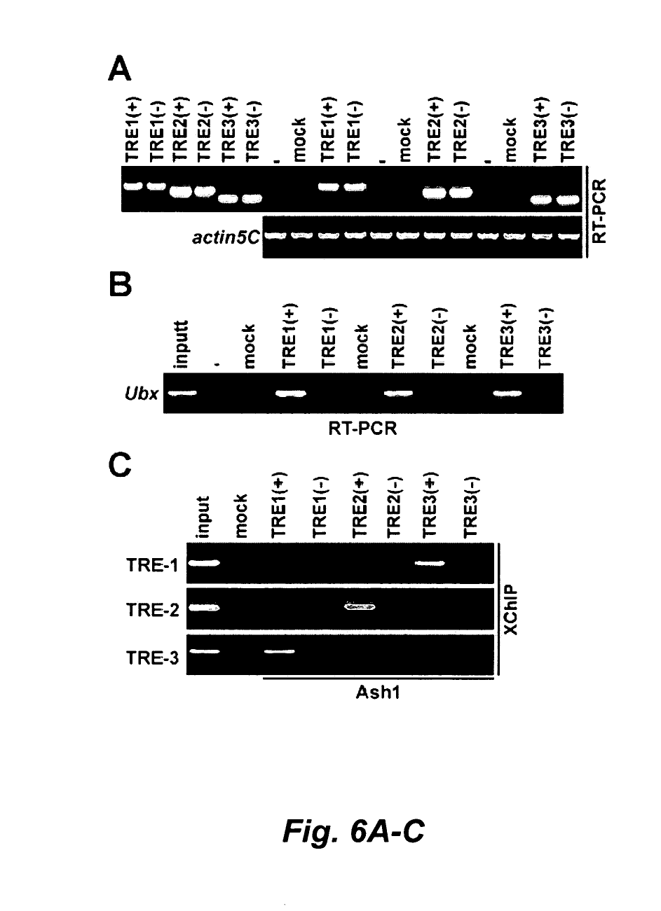

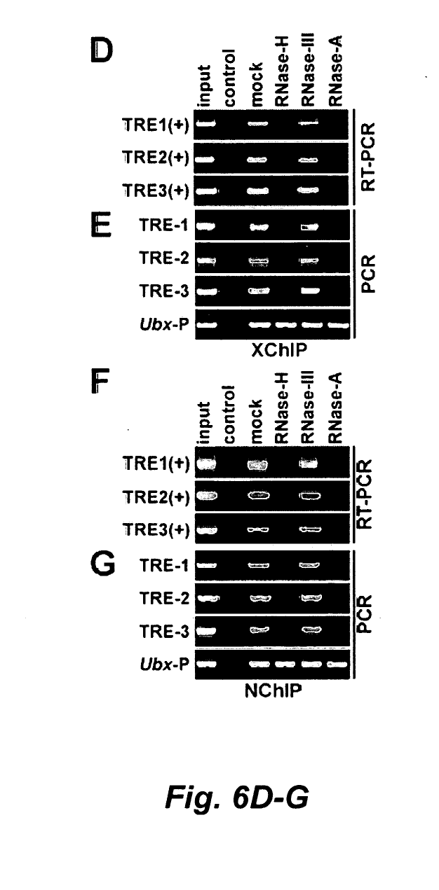

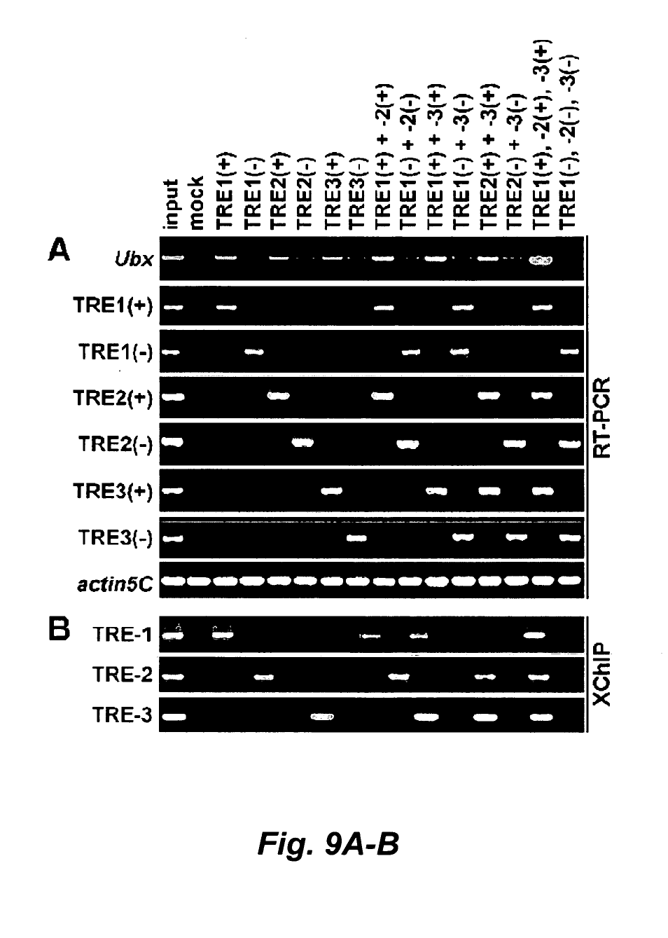

To dissect the role of TRE transcripts in Ubx transcription, the question of whether transiently transcribed TRE transcripts could restore the recruitment of Ash1 to Ubx TREs and Ubx expression in S2 cells was examined. S2 cells express Ash1 but lack endogenous TRE transcripts. S2 cells were transiently transfected with plasmids transcribing the TRE transcripts or the corresponding anti-sense RNAs and a control plasmid, expressing green fluorescent protein (GFP), to monitor transfection efficiency. Sixty hours after transfection, cells were harvested and used as a source for RNA and native as well as cross-linked chromatin. Isolated RNA was reverse transcribed, and PCR determining the amount of actin5C cDNA was used to standardize generated cDNA pools. In PCR assays, Ubx transcription was undetectable in wild-type S2 cells and cells transiently transcribing the anti-sense strand of TRE1, -2, and -3 or mdu (FIGS. 6A,B). In contrast, Ubx transcription was activated in the presence of sense TRE1(+), TRE2(+), and TRE3(+) (FIGS. 6A,B). Most interestingly, Ubx expression was significantly enhanced in cells transcribing 2 or all 3 TRE transcripts, which indicates that TRE transcripts activate Ubx expression in an additive or cooperative fashion (FIG. 9). The data indicate that transiently transcribed TRE transcripts can restore Ubx expression in S2 cells.

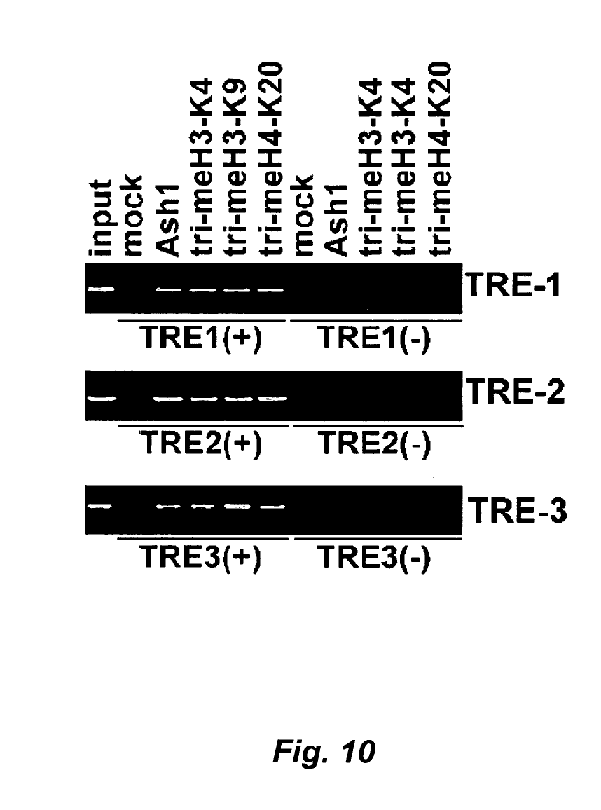

Next, XChIP was used to determine whether the rescue of Ubx transcription by transient TRE transcripts coincides with the recruitment of Ash1 to Ubx TREs. In vivo cross-linked chromatin was isolated from wild-type S2 cells and cells transiently transcribing one or multiple TRE transcripts and control RNAs (FIG. 6C; FIG. 9). Chromatin was sheared and immunoprecipitated with antibodies to Ash1, the Ash1 histone methylation pattern and rat serum. PCR detected the presence of TREs in precipitated DNA pools. Ash 1 was not detected at the TREs of transcriptionally silent Ubx in mock transfected cells transcribing mdu or the anti-sense TRE RNAs (FIG. 6C). In contrast, Ash1 and the Ash1 histone methylation pattern were detected at the Ubx TREs in cells transcribing TRE1 (+), TRE2(+), and/or TRE3(+) (FIG. 6C; FIG. 10). Remarkably, each of the 3 TRE transcripts facilitated the association of Ash1 only with the corresponding template TRE but not with other TREs, which provides evidence that TRE transcripts nucleate the recruitment of Ash1 to the corresponding TRE in chromatin.

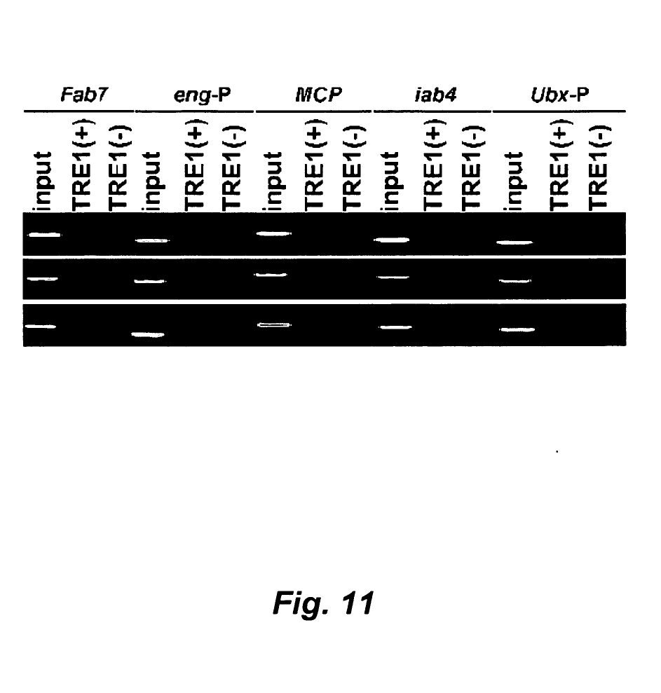

To verify the specificity of the described recruitment, the question of whether TRE transcripts facilitate recruitment of Ash1 to cellular memory elements (CMM) containing CEs and genes other than Ubx was investigated. In XChIP assays, Ash1 was not detected at Drosophila genes and CMM such as MCP and Fab7 in S2 cells transcribing TRE1(+), TRE2(+), or TRE3(+) (FIG. 11) (12,13). Thus, TRE transcripts facilitate Ash1 recruitment to the corresponding TRE template DNA rather than in a global fashion.

NChIP and XChIP were used to assess whether transiently transcribed TRE transcripts associate with TREs and Ash1 in chromatin. Native chromatin was isolated from wild-type S2 cells and S2 cells transiently transcribing all three TRE transcripts and anti-sense TRE transcripts as control. Purified chromatin was sheared, and treated with BSA (mock) and RNase-A, -III, or -H. One half of the treated chromatin was cross-linked. Both native and cross-linked chromatin were immunoprecipitated with antibodies to Ash1 and control antibodies (rat serum). Immunoprecipitates were divided in half to purify precipitated DNA and RNA. PCR and RT-PCR detected the presence of TREs and TRE transcripts, respectively, in precipitated nucleic-acid pools. Ash1 did not associate with TRE transcripts (FIGS. 6D,F) and TREs (FIGS. 6E,G) in mock-treated cross-linked (FIGS. 6D,E) and native chromatin (FIGS. 6F,G) prepared from wild type S2 cells and S2 cells transcribing control RNA. In contrast, Ash1 retained TREs and TRE transcripts in S2 cells co-transcribing TRE1(+), TRE2(+) and TRE3(+) (FIG. 6D-G).

The association of Ash1 with TREs and TRE transcripts was attenuated by RNase-A and —H but not RNase-III (FIG. 6D-G). RNase treatment did not abolish the association of TBP with the Ubx promoter. These results indicate that Ash1 associates with TRE transcripts and TREs in vivo and provide evidence that TRE transcripts bridge the association of Ash1 with TREs.

The disruption of TRE-Ash1 interactions by RNase-A indicates that single stranded motifs in TRE transcripts contribute the association of TRE transcripts and Ash1. In addition, attenuation of the association of Ash1 with TREs and TRE transcripts by RNase-H strongly suggests that transiently transcribed TRE transcripts hybridize with TREs and supports a model in which TRE transcripts are retained at TREs though RNA-DNA hybridization. In summary, the data provide evidence that non-coding Ubx TRE transcripts facilitate activation of Ubx expression by recruiting Ash1 to the TREs of Ubx.

### Discussion

The ability of proteins to recognize and bind target genes in chromatin represents one of the most fundamental mechanisms for the execution of DNA-dependent events. Different mechanisms underlying the recruitment of epigenetic regulators to chromatin have been described. In addition to binding to specific DNA target sequences, DNA-bound epigenetic activators and repressors can recruit additional epigenetic regulators to target genes through protein-protein interactions or by representing integral subunits of large epigenetic regulatory protein complexes (14,27,28). Third, recruitment of the epigenetic repressor Polycomb to chromatin involves the interaction of the repressor with methylated lysine 27 in H3 (28).

The data in this study reveal a novel role of non-coding TRE transcripts in epigenetic activation. The elucidation of this role is a based on results indicating that 1) the TREs and Ubx are transcribe in an identical tissue-specific pattern, 2) the epigenetic activator Ash1 associates with TRE transcripts in vitro and in vivo, 3) TRE transcripts mediate recruitment of Ash1 to TREs in vivo, and 4) transient TRE transcripts rescue Ubx expression in S2 cells. The data indicate that tissue-specific, non-coding TRE transcripts tether the epigenetic regulator Ash1 to Ubx.

Non-coding RNAs play an important role protein in the recruitment of proteins in several epigenetic phenomena. Although small (<25 nt) interfering RNAs (siRNAs) have originally been identified as posttranscriptional regulators of protein synthesis and stability in RNA interference (RNAi), recent studies have linked siRNAs with heterochromatin formation and transcriptional silencing of transgenes and transposons (30,31). SiRNAs facilitate the recruitment of HMTs and DNA methyltransferases to chromatin (32,33). In Schizosaccharomyces pombe, heterochromatic silencing is initiated by the recruitment of the RNA-induced initiator of transcriptional gene silencing complex (RITS) that contains an siRNA component which is essential for the recruitment of RITS to heterochromatic loci (32). The inability, however, of RNase-III, the key enzyme of the RNAi machinery, to process TRE transcripts into siRNAs and the interaction of Ash1 with full-length TRE transcripts in chromatin strongly argues against the involvement of the RNAi machinery in the described RNA-dependent recruitment of Ash1 to chromatin.

Long (>1000 nt) ncRNAs are key players in imprinting and gene dosage compensation (16,30,34). Diploid organisms have evolved gene dosage compensation mechanisms to equalize the disastrous differences in gene dosage resulting from the unequal distribution of sex chromosomes. In Drosophila, gene dosage compensation is achieved by a global 2-fold up-regulation of transcription from the male X chromosome and depends on the activity of the dosage compensation complex (DCC) that contains male-specific proteins as well as two ncRNAs, RNA on X1 (rox1), and RNA on X 2 (rox2) (16). Rox1 and rox2 are functionally redundant and transcribed by single-copy genes loci that in addition to approximately 30 other loci serve as chromatin entry sites (CEEs) for the DCC on paternal X chromosomes (16,30). Rox1 and Rox2 facilitate the assembly and recruitment of the DCC to CEEs (16). In mammals, transcription and spreading of Xist RNA culminates in X chromosome inactivation (18). Current models propose that the retention of long and small ncRNAs involves their interaction with proteins, nascent transcripts at template DNA or the template DNA itself (30,35). The observed attenuation of the association between TRE transcripts and TREs by RNase-H provides strong evidence that TRE transcripts are retained at TREs through hybridization with the corresponding template DNA. Because none of the known DNA repair systems targets DNA-RNA hybrids, RNA-DNA hybrids represent stable molecular entities that, in general, may anchor ncRNAs at corresponding DNA templates in chromatin (36).

Most known RNA-binding motifs identified in proteins bind single-stranded nucleotides in their corresponding target RNA (37). The interaction of Ash1 with ncRNA in vitro and the attenuation of the Ash1-TRE association by RNase-A indicate that Ash1 associates with single-strand RNA motifs protruding from the DNA-RNA hybrid rather than the DNA-RNA hybrid itself.

Computational sequence comparison revealed that the 3 TRE transcripts of Ubx do not share common sequence motifs. This is not surprising, since the functionally redundant rox RNAs and functionally identical regions in Xist, which are required for chromatin localization and protein recruitment, lack identifiable sequence motifs (30). Because many RNA-protein interactions are facilitated by distinct RNA secondary structures, the interaction of Ash1 with TRE transcripts might be mediated by secondary RNA structures rather than sequence motifs. In addition, the specificity of RNA-protein interactions is generated by induced-fit mechanisms. For example, human U1A, a member of the RNA recognition motif (RRM) family of RNA binding proteins, can bind different target RNAs (38). The initial interaction of U1A with target RNA depends on the presence of a minimal single-stranded RNA motif in a loop structure. This initial contact triggers complex, extensive conformational changes in both U1A and the target RNA that culminate in the specific intermolecular recognition of target RNAs by U1A.

Rox1 and rox2 RNAs transcribed from autosomes can localize to and mediate gene dosage compensation on the male X, which indicates that the chromatin entry of rox RNAs does not depend on CEE transcription in cis (39). Thus, the association of transiently transcribed TRE transcripts with TREs in S2 cells strongly suggests that TREs function as CEE for the corresponding TRE transcripts and that the transcription and CEE activity are functionally separated. The same CEE-activity may be responsible for the retention of nascent TRE transcripts at TREs. The ability of transgenic TRE transcripts to hybridize with transcriptionally inactive TREs requires local melting of DNA that, for example, can result from a very low, undetectable transcriptional activity of the apparently silent TREs or may occur during DNA replication. The latter hypothesis is supported by the observation that transient TRE transcripts require more than 48 h -during which S2 cells divide 3-4 times, to support Ubx transcription

Cumulatively, these results indicate that RNAs transcribed from the TREs of Ubx are retained at TREs through DNA-RNA interactions and provide a scaffold that is recognized and bound by Ash1. The tissue-specific transcription of other Drosophila CEs and evolutionary conservation of epigenetic regulators raise the possibility that ncRNAs may play a general role in the recruitment of epigenetic activators to target genes in metazoans.

### Example 2

### Non-Coding TRE1-RNA Mediates Transcription Activation by Ash1

### TRE1-RNA Mediates Transcription Activation by Ash1 in S2 Cells

To assess whether the transient transcription of TRE1-RNA restores recruitment of Ash1 and transcription activation of Ubx in S2 cells, the proprietary cell assay system described above was employed. Briefly, this system is based on the TET-on/TET-off system and transcribes TRE transcripts in S2 cells under control of the TET-transactivator (TET-VP16). Stable S2 cell lines [S2-tetO-TRE-1, -3, and -3] have been generated, which contain reporter plasmids transcribing the leading strand of TRE-1, -2, or -3 under control of the TET-transactivator. The reporter genes consist of 7 tetO-sites, a minimal promoter (TATA), TRE cDNA and flanking insulators. S2-tetO-TRE cells were transfected with plasmids expressing the TET-transactivator and EGFP. 60 h after transfection, EGFP-expressing cells were isolated by FACS and used as a source for chromatin and RNA.

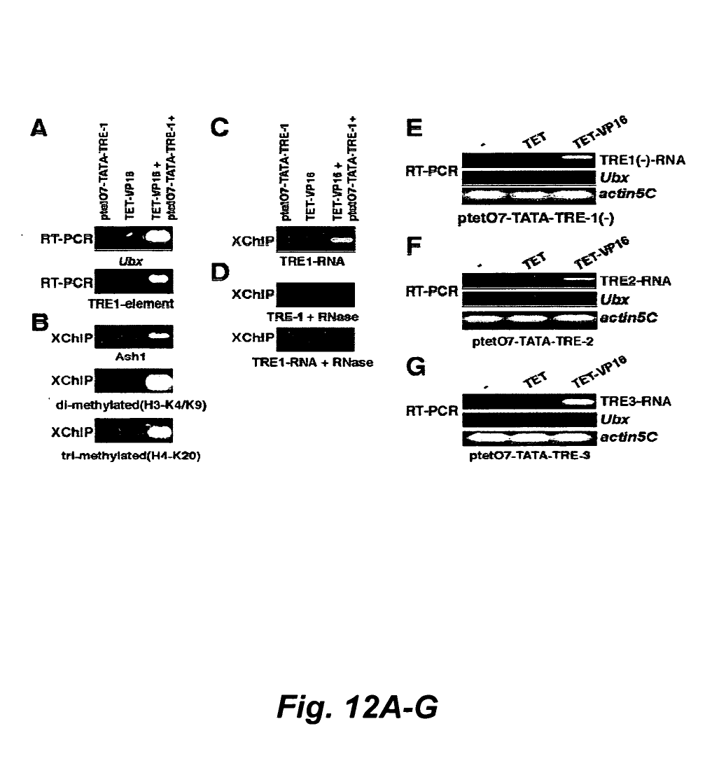

RT-PCR was used to monitor TRE-1 and Ubx transcription (FIG. 12A). XChIP using antibodies recognizing Ash1 or the Ash1 histone methylation pattern were used to detect the presence of Ash1 and its histone methylation pattern at the Ubx TRE-1 element. The results indicate that TRE1-RNA facilitates binding of Ash1 to TRE-1, activation of Ubx transcription and placement of the Ash1 histone methylation pattern at the reporter promoter (FIGS. 12A,B). To investigate whether Ash1 interacts with TRE1-transcript in vivo, in vivo cross-linked chromatin was immunoprecpiciptated using anti-Ash1 antibody and the precipitated RNA was purified. The RNA was reverse transcribed and used as a template for PCR that monitored the presence of TRE1-RNA in the precipitated RNA pools. Ash1 precipitated TRE1-RNA in S2 cells transcribing TRE-1 (FIG. 12C).

To support this result, RNase-assays were combined with XChIP to assess whether the interaction of Ash1 with TRE-1 is RNA-dependent. Chromatin was isolated from S2 cells transcribing TRE1-RNA and treated with an RNase-cocktail or a mock solution before cross-linking. XChIP using the monoclonal anti-Ash1 antibody was used to immunoprecipitate chromatin. Precipitated RNA and DNA were purified and used as a template for RT-PCR and PCR, respectively, to monitor the presence of the TRE-1 element and TRE1-RNA in precipitated nucleic acid pools. Ash1 did bind TRE-1 and TRE1-RNA in the absence but not in the presence of RNase, indicating that the Ash1 binds TRE1-RNA and that the recruitment of Ash1 to TRE-1 is RNA-dependent (FIG. 12D). In contrast, RNA representing the lagging strand of TRE-1 and the leading strand of TRE-2 and -3 transcripts did not mediate Ubx transcription (FIG. 12E-G). In summary, the results indicate that Ash1 binds TRE1-RNA in vivo and that this interaction plays an important role for the recruitment of Ash1 to the TRE-1 element in Ubx.

The recruitment of Ash1 by TRE1-RNA, which is transcribed in trans from transgenes, implies that the TRE1-RNA is recruited to the TRE-1 element of Ubx. This result argues for the possibility that TRE-1 as well as other TRE- and PRE-elements might contain specific RNA binding proteins that recruit or retain TRE- and PRE-transcripts.

### Miss-Transcription of TRE1-RNA Recruits Ash1 to Ubx in Drosophila Wing Imaginal Discs

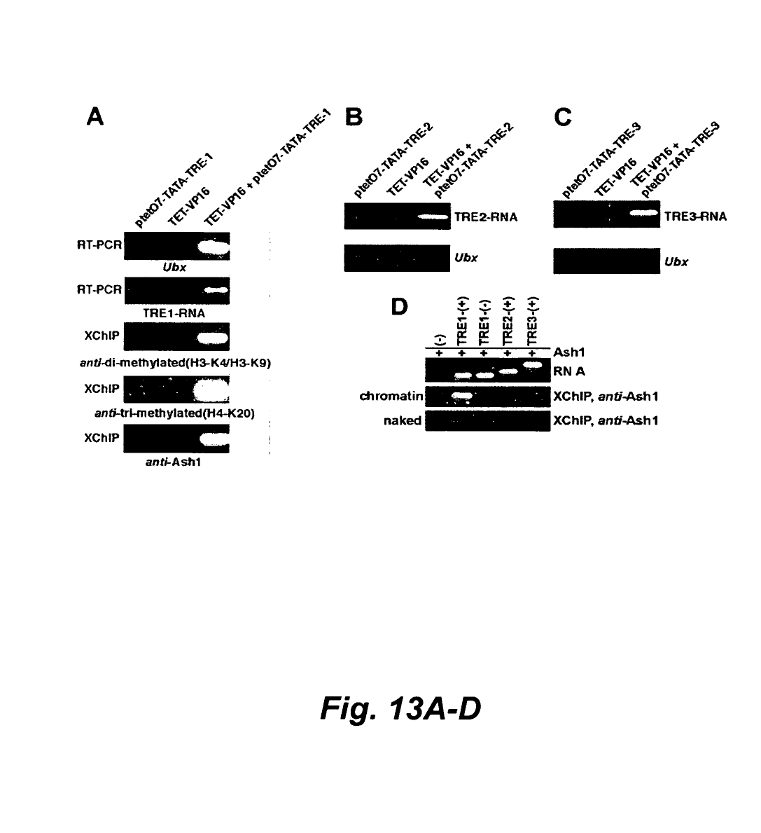

To assess the function of TRE1-RNA for Drosophila development, transgenic flies transcribing TRE-1 RNA in wing imaginal discs (wing discs) were generated. Although expressed in wing discs, Ash1 does not activate Ubx transcription in these tissues (FIG. 13A). RT-PCR assays indicate that TRE1-RNA is not detectable in wing discs, suggesting that the absence of this RNA prevents recruitment of Ash1 to Ubx and Ubx transcription (FIG. 13A). The described TET-on/TET-off strategy was used to generate effector and reporter fly strains that allow transcription of TRE1-RNA in wing discs under the control of the TET-transactivator (45). The effector strain contains a transgene that expresses the TET-transactivator under control of the decapletaplegic (dpp) enhancer/promoter in Drosophila wing discs (46). The reporter flies contain the ptetO7-TATA-TRE-1 reporter gene (see above). Transgenic flies were generated by P-element mediated transformation and identified by the presence of transgene-specific markers (45). Wing imaginal discs were isolated from 3rd instar larvae containing the effector gene, reporter gene, or both. Western blot analysis reveals that the TET-transactivator is expressed in wing discs (data not shown). RT-PCR assays indicate that TRE-1 and Ubx are transcribed in wing discs containing the effector and reporter genes but not in discs containing one of the transgenes (FIG. 13A). XChIP experiments indicate that Ubx transcription coincides with the presence of Ash1 and the corresponding histone methylation pattern at the transcriptionally active Ubx promoter (FIG. 13A). In contrast, the transcripts of TRE-2 and TRE-3 did not restore Ubx transcription (FIG. 13B-C), indicating that the transcription of TRE-1 RNA restores transcription activation by Ash1 in Drosophila.

These results imply that the interaction of Ash1 with TRE1-RNA mediates the recruitment of the epigenetic activator to target genes. However, the observed recruitment may be based on alternative mechanisms. HMT-assays indicated that the HMT-activity of Ash1 is not stimulated by TRE1-RNA (Sanchez-Elsner and Sauer, data not shown). To assess the possibility that the interaction of Ash1 with TRE1-RNA stimulates the DNA binding activity of Ash1 the interaction of Ash1 with naked DNA templates and chromatin templates was tested in the absence or presence of TRE1-RNA. A plasmid containing the 25 kb Ubx enhancer/promoter was used as the template for these assays. The reporter was packaged into chromatin using a Drosophila chromatin assembly system (46). XChIP monitored the binding of Ash1 to TRE-elements of naked and chromatin templates in the absence or presence of TRE1-RNA. Ash1 bound chromatin templates in the presence of TRE1-RNA. In contrast Ash1 did not bind the naked template in the absence or presence of TRE1-RNA. In summary, these results indicate that the interaction of Ash1 with TRE1-RNA mediates the recruitment of Ash1 to chromatin but does not induce a DNA binding activity of Ash1 (FIG. 13D).

### Example 3

### The Oncoproteins MLL and E(Z) Bind Hox Genes in an RNA-Dependent Fashion

Epigenetic regulators of the SET-module family are highly conserved. Like their Drosophila homologs, mammalian epigenetic regulators control the expression of homeotic genes (Hox genes) during development (47). Several mammalian epigenetic regulators of the SET-module family contribute to the expression of Hox genes (47). The epigenetic activator ‘Mixed Lineage Leukemia’ (MLL), the mammalian homologue of Drosophila Trithorax, activates the expression of several Hox genes during development. MLL has HMT-activity and methylation of H3-K4 by MLL has been correlated with transcription activation of several Hox genes. MLL has been closely connected with ‘Acute Myeloid Leukemia’ (AML) and ‘Acute Lymphoblastic Leukemia’ (ALL). Several different forms of MLL have been described in AML- and ALL-cells. The most predominant are fusion proteins between MLL and at least 30 different partners. These fusion proteins lack the COOH-terminal region of MLL including the SET-module. Other types of mutant MLL proteins present in AML and ALL-cells contain the SET-module, but lack a PHD-finger or contain duplications of the NH₂-terminal region suggesting that the SET-module contributes to the development of specific subtypes of ALL or AML.

The SET-module repressor ‘Enhancer-of-Zeste’ (EZH2) represses the transcription of several Hox genes during development. EZH2 has HMT-activity and methylates H3-K9 and/or H3-K27. H1stone methylation by EZH2 has been correlated with silencing, imprinting and X-chromosome inactivation. Aberrant EZH2-activity has been correlated with various cancers including prostate, lung and breast cancer.

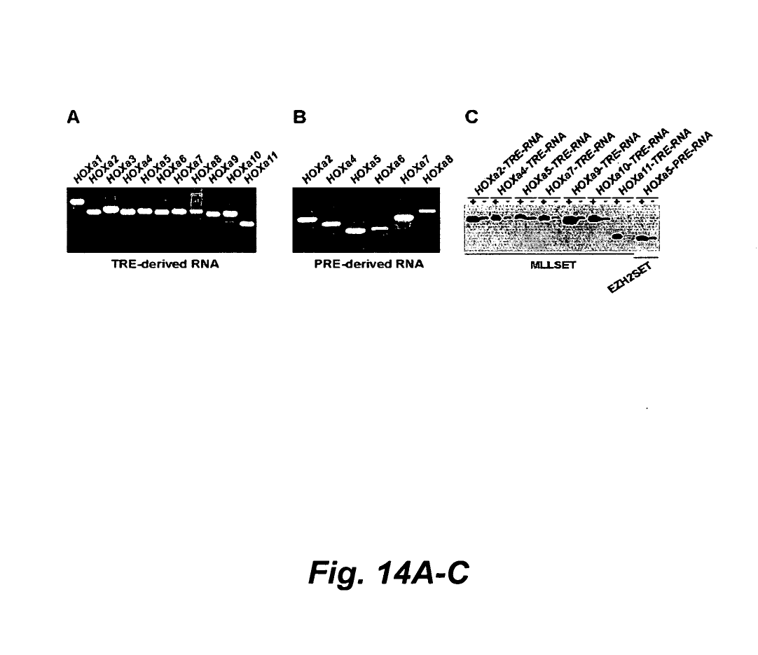

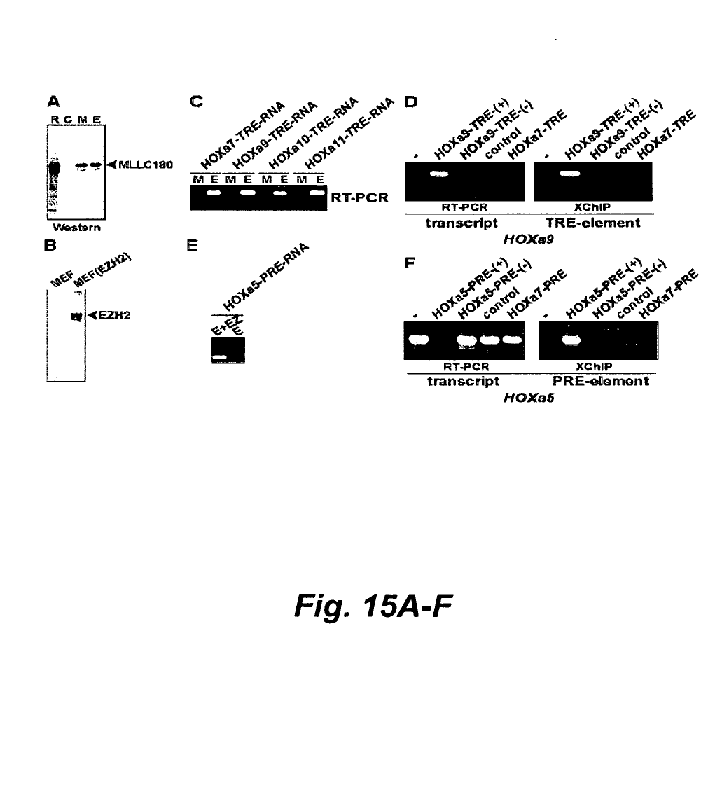

Notably, the cancerous activity of both MLL and EZH2 has been correlated with the dys-regulation of Hox gene expression. Despite the presence of putative DNA-motifs in MLL and EZHZ, it remained unknown how both HMTs recognize and bind target genes. The result that Ash1 binds TRE1-RNA lead to an investigation of whether ncRNA transcribed from the TRE- and PRE-elements of MLL and EZH2 target genes facilitates the recruitment of the epigenetic regulators to target genes. Because the TRE- and PRE-elements in MLL and EZH2 target genes remained mysterious two different strategies were used to identify the target regions of MLL and EZH2 in the cis-regulatory region of Hox genes. The first strategy is based on XChIP, the second strategy is based on computational approaches using consensus DNA sequences of Drosophila TRE- and PRE-elements (Sanchez-Elsner and Sauer, data not shown). The approaches resulted in the identification of 16 putative TRE and PRE-elements present in Hox genes that are targeted by MLL or EZH2. To assess whether the putative TRE- and PRE-elements are transcribed in vivo, RT-PCR was used to detect TRE- and PRE-transcripts in commercially available mouse cDNA libraries. Transcripts for 11 of the putative TREs and for 4 of the putative PREs present in Hox genes were detected (FIG. 14A-B). For five of these, start and endpoints in the Hox gene cluster sequence (Pbumed Genbank accession no. NT_{—}039343) are shown in Table 4.

TABLE 4
Start and Endpoints of Putative TREs and PREs in HOX gene cluster¹

| | Starting | Ending |
|---|---|---|
| Hox Gene | Nucleotide | Nucleotide |
| HoxA5 | 110021 | 111682 |
| HoxA7 | 91755 | 98623 |
| HoxA9 | 86300 | 89763 |
| HoxA11 | 68723 | 70023 |
| HoxC8 | 266167 | 270603 |

¹Numbered according to Pbumed Genbank accession no. NT 039343.

To assess whether MLL and EZH2 interact with TRE- and PRE-transcripts respectively, protein:RNA interaction assays were performed as described above but using recombinant MLL and EZH2-derivatives, which were expressed in and purified from Sf9 cells. Full-length MLL (data not shown) as well as the SET-module of the HMT did bind the transcripts of several Hox TREs including the transcript of Hoxa9 (435 nt) (FIG. 14A) Similarly, full-length EZH2 (data not shown) and the SET-module of the HMT interacted with the transcript derived from the PRE-element of Hoxa5 (585 nt) (FIG. 14B). MLL- and EZH2-derivatives lacking the SET-module did not bind RNA (data not shown). In summary, the results indicate that mammalian members of the SET-module family of epigenetic regulators bind TRE and PRE transcripts.

### Example 4

### TRE and PRE Transcription in Drosophila

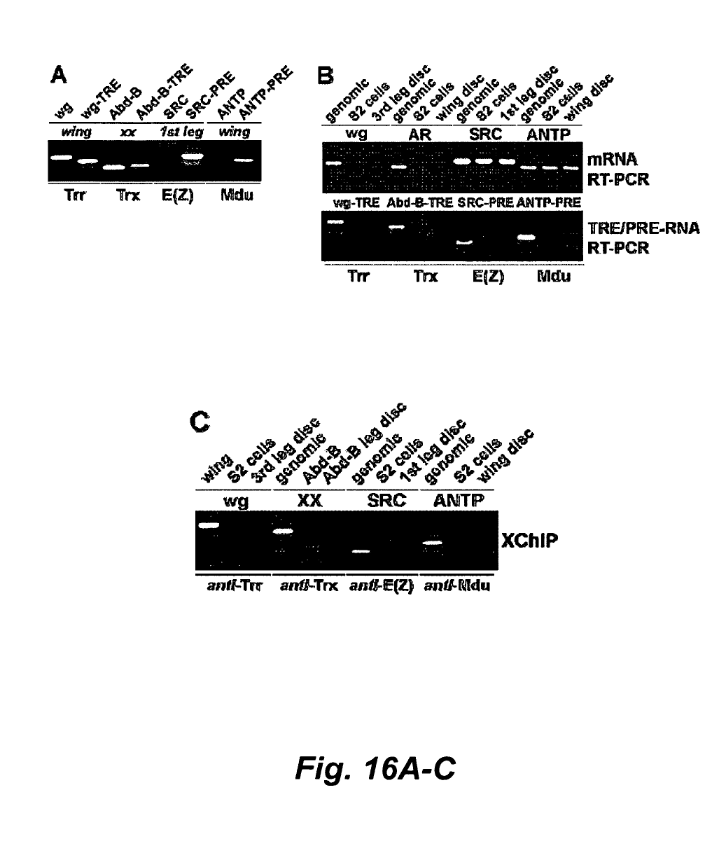

Protein:RNA interaction assays were used to assess the interaction of Drosophila SET-module epigenetic regulators with TRE- or PRE-transcripts. In addition to Ash1, Drosophila contains several epigenetic activators and repressors of the SET-module protein family: the activators trithorax (Trx) and Trr and the epigenetic repressors enhancer of Zeste [E(Z)] and Mdu. Trx activates the transcription of several homeotic genes in Drosophila e.g., abdominal B (Abd-B). Like Trr, transcription activation by Trx coincides with methylation of H3-K4 (33). Transcription repression by Mdu correlates with methylation of H3-K9 while repression by E(Z) coincides with methylation of H3-K27 (18). E(Z) represses the transcription of Sex-combs reduced (SCR) in 1st leg imaginal discs and Mdu represses the transcription of ANTP (FIG. 16). TRE-(TRX, Trr) and PRE-elements [Mdu, E(Z)] in the target genes of the regulators have been identified and transcription of these elements in vivo has been confirmed using RT-PCR (FIG. 16).

### Example 5

### TRE and PRE Transcription in Mammalian Cells

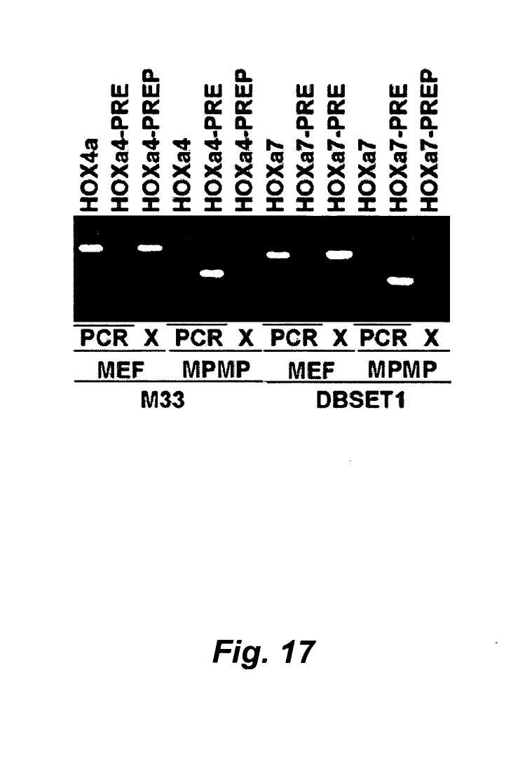

Like Drosophila, mammalian cells contain numerous members of the SET-module and chromodomain families of epigenetic regulators. To investigate whether the mammalian epigenetic repressors M33, (the mammalian homologue of PC) and SETDB1 (one of the mammalian homologues of Mdu) interact with PRE transcripts (18; Bermudez and Sauer, unpublished data), target genes and target PREs for the two repressors have been identified. RT-PCR has been used to confirm that the targeted PRE-elements are transcribed in vivo (FIG. 17).

### Example 6

### Screen to Identify Protein-ncRNA Interactions

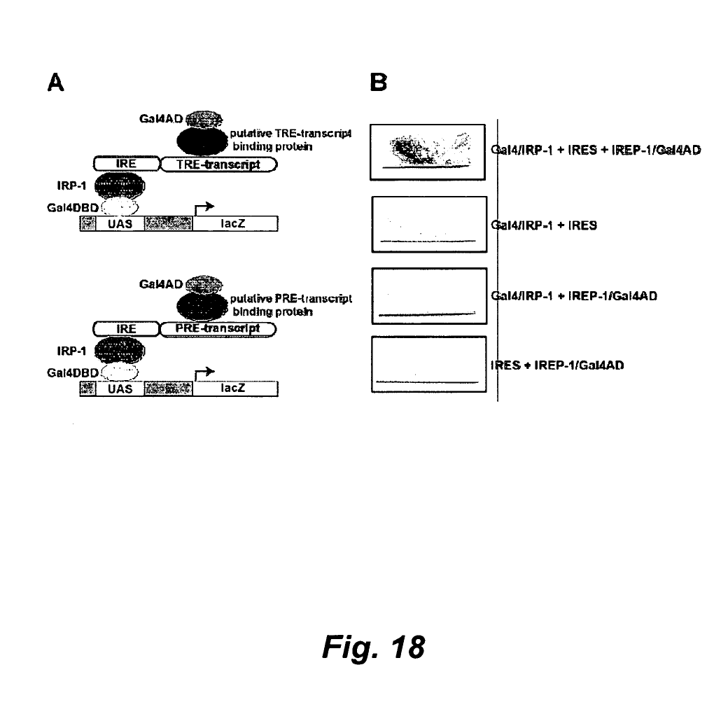

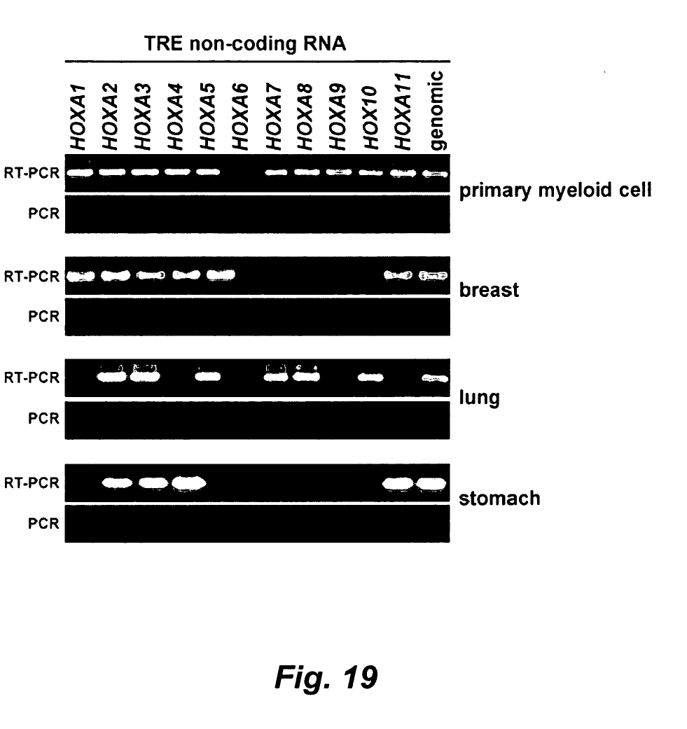

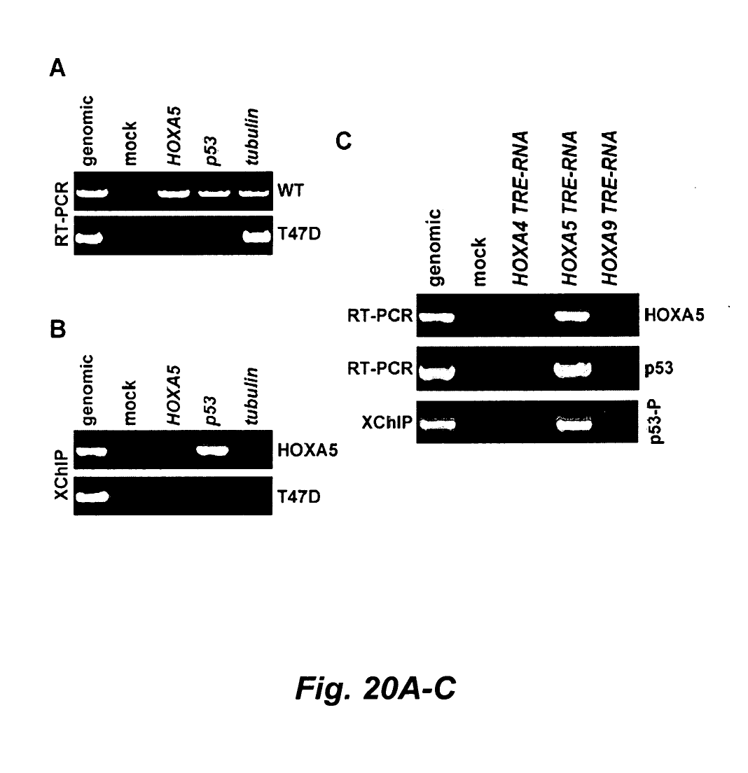

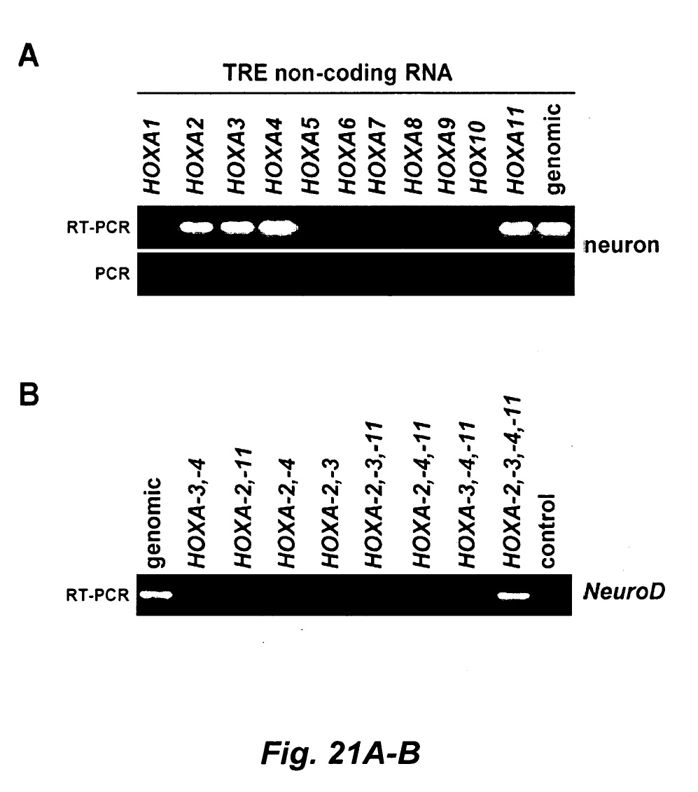

A modified version of the yeast two hybrid system (YTHS) is used to identify protein-ncRNA interations. The YTHS can detect protein:protein interactions in the context of the yeast cell. To identify proteins that bind TRE- or PRE-RNA, a YTHS that can detect RNA:protein interaction in cells has been established (FIG. 18A). This system employs a yeast cell that express a fusion protein consisting of the DNA binding domain of the yeast activator Gal4 (Gal4 DBD) and iron regulation protein 1 (IRP-1). IRP-1 binds an RNA motif termed an ‘iron response element’ (IRE), which is located in the 5′-prime untranslated region of the ferritin mRNA and represses translation of the ferritin transcript. In addition, the yeast cells transcribe a fusion-RNA consisting of three IRE elements and a TRE- or a PRE-transcript. The yeast cells are transformed with a yeast plasmid expression library expressing fusion proteins consisting of Drosophila or mouse proteins and the activation domain of Gal4 (Gal4AD). The interaction of Gal4 DBD/IRP— 1 with IREs and a Drosophila or mouse fusion protein with the TRE- or PRE-transcript present in the fusion-RNA will reconstitute a transcription factor that mediates Gal4-dependent transcription activation in yeast (FIG. 18B). The functionality of this system has been confirmed by assessing the recruitment of an IRP-1/Gal4AD fusion protein to the Gal4 DBD/IRP-1/IRE complex in yeast. Transcriptional activation of Gal4 target genes was observed in the presence of all three components but not in cells expressing only one or two of the three involved components (FIG. 8B).

To identify the protein(s) that interact with TRE- and PRE-transcripts, cells expressing Gal4 DBD/IRP-1 and IRES-TRE- or IRES-PRE-RNA are transformed with commercially available Drosophila and mouse expression plasmids. The interaction of fusion protein with a Gal4 DBD/IRP-1/IRES-TRE-RNA or Gal4 DBD/IRP-1/IRES-PRE-RNA complex, but not Gal4 DBD/IRP-1 or control fusion-RNAs (e.g., IRE-lacZ), restores Gal4-dependent transcriptional activation in yeast cells. The plasmid(s) encoding the relevant fusion protein(s) are then isolated and sequenced to reveal the identity of the putative TRE- or PRE-transcript binding protein(s). The interaction of the identified protein(s) with TRE- or PRE-RNA and the functional importance of the protein for the recruitment of epigenetic activators and repressors to target genes are then determined as described above. This will allow the identification and characterization of the RNA binding protein(s) that retain TRE- or PRE-transcripts at their template DNA.

### REFERENCES

1.) R. Jaenisch, A. Bird. Nat. Genet. 33 Suppl, 245-254 (2003).
2.) B. M. Turner. Cell 111, 285-291 (2002).
3.) V. Orlando. Cell 112, 599-606 (2003).
4.) L. Ringrose, R. Paro. Annu. Rev. Genet. 38, 413-43 (2004).
5.) A. Breiling, A., V. Orlando, V. Nat. Struct. Biol. 9, 894-896 (2002)
6.) P. B. Becker, W. Horz. Annu. Rev. Biochem. 71, 247-273 (2002). Epub 2001 Nov. 9.
7.) T. Jenuwein, C. D. Allis (2001). Science 293, 1074-1080 (2001).
8.) R. Cao, Y. Zhang Y. Curr. Opin. Genet Dev. 2, 155-164 (2004).
9.) C. Beisel, A. Imhof, J. Greene, E. Kremmer, F. Sauer. Nature 419 857-862 (2002).
10.) N. Tripoulas, D. LaJeunesse, J. Gildea, A. Shearn Genetics 143, 913-928 (1996).
11.) D. LaJeunesse D., A. Shearn. Mech. Dev. 53, 123-39 (1995).
12.) S. Schmitt, M. Prestel, R. Paro. Genes Dev. 19, 697-708 (2005). Epub 2005 Mar. 1.
13.) G. Rank, M. Prestel, R. Paro. Mol. Cell. Biol. 22, 8026-34 (2002).
14.) J. Dejardin, A. Rappailles, O. Cuvier, C. Grimaud, M. Decoville, D. Locker, G. Cavalli. Nature 434, 533-538 (2005).
15.) Y. Zhang, D. Reinberg. Genes Dev. 15, 2343-2360 (2001).
16.) A. Akhtar. Curr. Opin. Genet. Dev. 13, 161-169 (2003).
17.) A. Wutz. Bioessays 25, 434-442 (2003).
18.) E. Heard. Curr. Opin. Cell Biol. 16, 247-255 (2004).
19.) M. A. Matzke, J. A. Birchler. Nat. Rev. Genet. 6, 24-35 (2005).
20.) J. W. Little, C. A. Byrd, D. L. Brower. Genetics 120, 181-198 (1990).
21.) T. Rozovskaia et al. Mol. Cell. Biol. 19, 6441-6447 (1999).
22.) C. H. Martin et al. Proc. Natl. Acad. Sci. USA 92, 8398-8402 (1995).
23.) H. D. Lipshitz, D. A. Peattie, H. S. Hogness. Genes Dev. 1, 307-322 1987).
24.) W. A. Krajewski, T. Nakamura, A. Mazo, E. Canaani. Mol. Cell. Biol. 25, 1891-1899 (2005).
25.) S. R. Albright, R. Tjian. (2000). Gene 242, 1-13 (2000).
26.) T. Maile, S. Kwoczynski, R. J. Katzenberger, D. A. Wassarman, and F. Sauer. Science 304, 1010-1014 (2004).
27.) J. A. Simon, J. W. Tamkun. Curr. Opin. Genet. Dev. 12, 210-218
28.) B. Czermin, R. Melfi, D, McCabe, V. Seitz, A. Imhof, A., and V. Pirrotta, V. Cell 111, 185-196 (2002).
29.) R. Cao et al. Science 298, 1039-1043 (2002). Epub 2002 Sep. 26.
30.) E. J. Sontheimer. Nat. Rev. Mol. Cell. Biol. 6, 127-138.
31.) V. Schranke, R. Allshire. Curr. Opin. Gen. Gev. 14, 174-180 (2004).
32.) A. Verdel et al. Science 303, 672-676 (2004).
33.) M. J. O'Neill. Hum. Mol. Genet. 14 Spec No 1:R113-20 (2004).
34.) S. W. -L. Chan, D. Zilberman, Z. Xie, L. K. Johansen, J. C. Carrington, S. E. Jacobsen. Science 303, 1336.
35.) J. C. Rice, S. I. Grewal. Curr. Opin. Cell Biol. 16, 230-238.
36.) M. Christmann M, M. T. Tomicic, W. P. Roos, B. Kaina. Toxicology 193, 3-34 (2003).
37.) Y. Chen, G. Varani. FEBS J. 272, 2088-2097 (2005).
38.) F., H. -T. Allain, P. W. A., Howe, D. Neuhaus, G. Varani. EMBO J. 16, 5764-5772 (1997).
39.) V. H. Meller, B. P. Rattner. EMBO J. 21, 1084-91 (2002).
41.) M. Koelle, M., D. Hogness, D. D. I. N. 8 (1992).
42.) S. K. Hansen, R. Tjian, Cell 82, 565 (1995).
43.) F. Sauer, H. Jackle. Nature 353: 563-565 (1991).
44.) A. -D. Pham, F. Sauer. Science 289, 2357-2360 (2000).
45.) Karess, R. E., and Rubin, G. M. (1984). Analysis of P transposable element functions in Drosophila. Cell 38, 135-146.
46.) Johnston, L. A., and Schubiger, G. (1996). Ectopic expression of wingless in imaginal discs interferes with decapentaplegic expression and alters cell determination. 122, 3519-3529.
47.) Kmita, M., and Duboule, D. (2003) Organizing axes in time and space; 25 years of collinear thinking. Science 301, 331-333.

It is understood that the examples and embodiments described herein are for illustrative purposes only and that various modifications or changes in light thereof will be suggested to persons skilled in the art and are to be included within the spirit and purview of the application and scope of the appended claims. All publications, patents, and patent applications cited herein are hereby incorporated by reference in their entirety for all purposes.

1. A method of regulating transcription of a gene that is a target for an epigenetic regulator, the method comprising contacting cells comprising the gene and the epigenetic regulator with an effective amount of a modulator, wherein:
the gene comprises a cis-regulatory region including a chromosomal element (CE) for the epigenetic regulator;
the CE comprises a sequence that is a template for a non-coding polynucleotide;
the modulator alters the level of:
the non-coding polynucleotide;
the specific binding of the non-coding polynucleotide to the target gene; and/or
the specific binding of the epigenetic regulator to the non-coding polynucleotide; and
an effective amount is an amount sufficient to regulate transcription of the gene.

2. The method of claim 1, wherein the cells comprise mammalian cells.

3. The method of claim 2, wherein the mammalian cells comprise human cells.

4. The method of claim 1, wherein the gene that is a target for the epigenetic regulator comprises a homeotic gene.

5. The method of claim 4, wherein the homeotic gene comprises a gene selected from the group consisting of Ultrabithorax (Ubx), abdominal B (abd-B), wingless (wg), Sex-combs reduced (SCR), Antennapedia (ANTP), a Hox gene, and an ortholog thereof.

6. The method of claim 1, wherein the epigenetic regulator comprises a histone methylransferase.

7. The method of claim 1, wherein the epigenetic regulator comprises a SET-module.

8. The method of claim 1, wherein the epigenetic regulator activates transcription of the target gene.

9. The method of claim 8, wherein the epigenetic regulator comprises a regulator selected from the group consisting of Trithorax (Trx), Trithorax-related (Trr), absent small and homeotic discs (Ash1), human Trx, human Ash1, human Ash2, Mixed Lineage Leukemia (MLL), MLL-related (MLL-1, MLL-2, MLL-3, MLL-4, MLL-5), ALL-1, ALL-2, ALL-3, ALL-4, ALL-5, and an ortholog thereof.

10. The method of claim 1, wherein the epigenetic regulator represses transcription of the target gene.

11. The method of claim 10, wherein the epigenetic regulator comprises a regulator selected from the group consisting of D. melanogaster Enhancer of Zeste (E(Z)), Polycomb (PC), Medusa (Mdu), Su(var)3-5, Su(var)3-7, Su(var)3-9, Su(var)3-6, Su(var)2-1, Su(var)2-10, Su(var)3-3, mammalian Enhancer of Zeste (EZH2), M33, SETDB1, ENX-2, mammalian SUV39H1, SUV39H2, and an ortholog thereof.

12. The method of claim 1, wherein the non-coding polynucleotide comprises non-coding RNA.

13. The method of claim 1, wherein the modulator alters the level of the non-coding polynucleotide.

14. The method of claim 1, wherein the modulator alters the level of the specific binding of the non-coding polynucleotide to the target gene.

15. The method of claim 1, wherein the modulator alters the level of the specific binding of the epigenetic regulator to the non-coding polynucleotide.

16. The method of claim 1, wherein the modulator reduces said level.

17. The method of claim 16, wherein the epigenetic regulator comprises a transcriptional activator, and the modulator represses transcription of the target gene.

18. The method of claim 16, wherein the epigenetic regulator comprises a transcriptional repressor, and the modulator activates transcription of the target gene.

19. The method of claim 1, wherein the modulator increases said level.

20. The method of claim 19, wherein the epigenetic regulator comprises a transcriptional activator, and the modulator activates transcription of the target gene.

21. The method of claim 19, wherein the epigenetic regulator comprises a transcriptional repressor, and the modulator represses transcription of the target gene.

22. The method of claim 1, wherein the modulator modulates cell proliferation and/or cell differentiation.

23. The method of claim 1, wherein the cells are in vitro.

24. The method of claim 23, wherein the cells are removed from a patient having a condition selected from the group consisting of cancer, neurodegenerative disease, paralysis, diabetes, burn, tissue failure, organ failure, osteoporosis, muscular dystrophy, and wound, contacted with the modulator, and then reimplanted into the patient.

25. The method of claim 1, wherein the cells are in vivo.

26. The method of claim 25, wherein said contacting is performed by administering a composition comprising the modulator to a subject having a condition treatable by modulation of cell proliferation and/or cell differentiation.

27. The method of claim 25, wherein said contacting is performed by administering a composition comprising the modulator to a patient having a condition selected from the group consisting of cancer, neurodegenerative disease, paralysis, diabetes, burn, tissue failure, organ failure, osteoporosis, muscular dystrophy, and wound.

28. The method of claim 22, wherein the cell is selected from the group consisting of a cancer cell, a stem cell, and a dormant cell.

29. The method of claim 28, wherein the cell comprises a stem cell, and the transcription of one or more genes that is/are a target for one or more epigenetic regulators is regulated to induce the stem cell to differentiate.

30. A method of characterizing the transcriptional activity of a gene that is a target for an epigenetic regulator in a biological sample comprising the gene and the epigenetic regulator, wherein:
the gene comprises a cis-regulatory region including a chromosomal element (CE) for the epigenetic regulator; and
the CE comprises a sequence that is a template for a non-coding polynucleotide;
the method comprising determining whether the non-coding polynucleotide is present in the biological sample.

31-49. (canceled)

50. A method of screening for a chromosomal element (CE) for an epigenetic regulator of a target gene, wherein the CE comprises a sequence that is a template for a non-coding polynucleotide;
the method comprising determining whether a sequence of a putative CE is transcribed in a cell.

51. The method of claim 50, wherein the putative template is identified by sequence comparison with a CE selected from tre1, tre2, and tre3.

52. A method of screening for a chromosomal element (CE) for an epigenetic regulator of a target gene, wherein the CE comprises a sequence that is a template for a non-coding polynucleotide;
the method comprising determining whether the epigenetic regulator is physically associated with a non-coding polynucleotide corresponding to a putative CE and/or physically associated with the putative CE.

53-54. (canceled)

55. A method of screening for a chromosomal element (CE) for an epigenetic regulator of a target gene, wherein the CE comprises a sequence that is a template for a non-coding polynucleotide;
the method comprising determining whether a non-coding polynucleotide corresponding to a putative CE mediates transcriptional regulation by the epigenetic regulator.

56-69. (canceled)

70. An isolated complex comprising an epigenetic regulator for a target gene, wherein the epigenetic regulator is specifically bound to a non-coding polynucleotide, and wherein:
the target gene comprises a cis-regulatory region including a chromosomal element (CE) for the epigenetic regulator; and
the CE comprises a sequence that is a template for the non-coding polynucleotide.

71-81. (canceled)

82. A method of screening for a modulator of transcription of a gene that is a target for an epigenetic regulator, wherein:
the gene comprises a cis-regulatory region including a chromosomal element (CE) for the epigenetic regulator;
the CE comprises a sequence that is a template for a non-coding polynucleotide;
the method comprising:
a) contacting a test agent with a mixture or cell comprising the non-coding polynucleotide and the CE and/or the epigenetic regulator, and
b) detecting the ability of the test agent to modulate specific binding of the non-coding polynucleotide to the CE and/or the epigenetic regulator.

83-100. (canceled)


SEQUENCE LISTING (54 sequences)

SEQ ID NO: 1 - RNA, 14 nt, Artificial
  Ribozyme cleavage site
nnnbngucnnnnnn

SEQ ID NO: 2 - DNA, 24 bp, Artificial
  PCR primer
ccggtacacgttattcacttcgac

SEQ ID NO: 3 - DNA, 20 bp, Artificial
  PCR primer
cggccctccatcaacgcttc

SEQ ID NO: 4 - DNA, 17 bp, Artificial
  PCR primer
atgaacagaagcagcag

SEQ ID NO: 5 - DNA, 22 bp, Artificial
  PCR primer
cggagcaatttgtcaccgcaag

SEQ ID NO: 6 - DNA, 20 bp, Artificial
  PCR primer
gctctcgctttacggcgcag

SEQ ID NO: 7 - DNA, 21 bp, Artificial
  PCR primer
ttgttgcatatgcaacccaag

SEQ ID NO: 8 - DNA, 21 bp, Artificial
  PCR primer
gatccgagcgagaaggctaac

SEQ ID NO: 9 - DNA, 20 bp, Artificial
  PCR primer
gtccccttctaacagccgtg

SEQ ID NO: 10 - DNA, 24 bp, Artificial
  PCR primer
cattgtgctcgggcactgattgaa

SEQ ID NO: 11 - DNA, 18 bp, Artificial
  PCR primer
ggcacgcactaaacccca

SEQ ID NO: 12 - DNA, 22 bp, Artificial
  PCR primer
ggcgttcggataatttggcctc

SEQ ID NO: 13 - DNA, 24 bp, Artificial
  PCR primer
gcgtcgaagtgaataacgtgtacc

SEQ ID NO: 14 - DNA, 26 bp, Artificial
  PCR primer
ccgggcgagtcaattaaatcaaatgg

SEQ ID NO: 15 - DNA, 22 bp, Artificial
  PCR primer
gagttccgtgattggattgccc

SEQ ID NO: 16 - DNA, 25 bp, Artificial
  PCR primer
cggcatcggttgtttgttgtttctg

SEQ ID NO: 17 - DNA, 21 bp, Artificial
  PCR primer
ccgcgtccgcaaaactagcaa

SEQ ID NO: 18 - DNA, 21 bp, Artificial
  PCR primer
cgttctggactccggcgatgg

SEQ ID NO: 19 - DNA, 21 bp, Artificial
  PCR primer
gtacttgcgctctggcggggc

SEQ ID NO: 20 - DNA, 21 bp, Artificial
  PCR primer
cgtacatgggggcggacatgc

SEQ ID NO: 21 - DNA, 21 bp, Artificial
  PCR primer
cctggggcatgaccccgccca

SEQ ID NO: 22 - DNA, 21 bp, Artificial
  PCR primer
gccatcgtcggcgagtacttc

SEQ ID NO: 23 - DNA, 21 bp, Artificial
  PCR primer
ggaataccggggtgaacccag

SEQ ID NO: 24 - DNA, 21 bp, Artificial
  PCR primer
ccgagttgtcgctcatggagg

SEQ ID NO: 25 - DNA, 21 bp, Artificial
  PCR primer
tcccgcatggcctgcgtgttg

SEQ ID NO: 26 - DNA, 21 bp, Artificial
  PCR primer
gtcctcaccggcgatcattcg

SEQ ID NO: 27 - DNA, 21 bp, Artificial
  PCR primer
gtcggttgcgggtggatcggc

SEQ ID NO: 28 - DNA, 21 bp, Artificial
  PCR primer
cggcagcgagcagggcaatct

SEQ ID NO: 29 - DNA, 21 bp, Artificial
  PCR primer
gaagtgggcactggcgcagac

SEQ ID NO: 30 - DNA, 20 bp, Artificial
  PCR primer
atggccaccggaatgccccc

SEQ ID NO: 31 - DNA, 21 bp, Artificial
  PCR primer
cgcctcagtcttgtagggctt

SEQ ID NO: 32 - DNA, 21 bp, Artificial
  PCR primer
gtggatctcgtcgtgctcgcc

SEQ ID NO: 33 - DNA, 21 bp, Artificial
  PCR primer
tgctggcggttccgggcgctt

SEQ ID NO: 34 - DNA, 21 bp, Artificial
  PCR primer
gcccgcctggatggcactccc

SEQ ID NO: 35 - DNA, 21 bp, Artificial
  PCR primer
ctcgtatccgccctggcttcc

SEQ ID NO: 36 - DNA, 21 bp, Artificial
  PCR primer
cgttctggactccggcgatgg

SEQ ID NO: 37 - DNA, 20 bp, Artificial
  PCR primer
gtacttgcgctctggcgggg

SEQ ID NO: 38 - DNA, 21 bp, Artificial
  PCR primer
ccatgatgaatttcccgcggc

SEQ ID NO: 39 - DNA, 20 bp, Artificial
  PCR primer
agcggtaaagcgctgagggc

SEQ ID NO: 40 - DNA, 23 bp, Artificial
  PCR primer
atcatatgactgcggccactacc

SEQ ID NO: 41 - DNA, 25 bp, Artificial
  PCR primer
caggatcatatggactcagttttgg

SEQ ID NO: 42 - DNA, 17 bp, Artificial
  PCR primer
tcaggtcaaacgcgtcg

SEQ ID NO: 43 - DNA, 22 bp, Artificial
  PCR primer
atttgtgtaaccgtgtgacggc

SEQ ID NO: 44 - DNA, 19 bp, Artificial
  PCR primer
acgcgtttgaccttgaggc

SEQ ID NO: 45 - DNA, 21 bp, Artificial
  PCR primer
acacatccacaagcggaccag

SEQ ID NO: 46 - DNA, 23 bp, Artificial
  PCR primer
ttgcaacatctataaaagggccg

SEQ ID NO: 47 - DNA, 21 bp, Artificial
  PCR primer
ttctttgacatttgccgtcgc

SEQ ID NO: 48 - DNA, 19 bp, Artificial
  PCR primer
aaacacgaatacaagcccg

SEQ ID NO: 49 - DNA, 24 bp, Artificial
  PCR primer
aatgctactgctctctaggccacg

SEQ ID NO: 50 - DNA, 20 bp, Artificial
  PCR primer
ttcaatcagtgcccgagcac

SEQ ID NO: 51 - DNA, 19 bp, Artificial
  PCR primer
ttcgcctgttgccttggcg

SEQ ID NO: 52 - DNA, 23 bp, Artificial
  PCR primer
aagcggaaaacgaaagagagcgc

SEQ ID NO: 53 - DNA, 20 bp, Artificial
  PCR primer
agcaaacatgttgcgagtgc

SEQ ID NO: 54 - protein, 15 aa, Drosophila melanogaster
RKTQQSSSSSTANST